(19) United States
(12) Patent Application Publication
(10) Pub. No.: US 2008/0113351 A1
(43) Pub. Date: May 15, 2008

(54) Polynucleotides for causing RNA interference and method for inhibiting gene expression using the same

(75) Inventors: Yuki Naito, Tokyo (JP); Masato Fujino, Tokyo (JP); Shinobu Oguchi, Tokyo (JP); Yukikazu Natori, Yokahama-Shi (JP)

Correspondence Address: BIRCH STEWART KOLASCH & BIRCH, PO BOX 747, FALLS CHURCH, VA, 22040-0747, US

(73) Assignee: alphaGEN Co., Ltd., Tokyo (JP)

(21) Appl. No.: 11/598,052

(22) Filed: Nov. 13, 2006

Related U.S. Application Data
(63) Continuation of application No. PCT/IB05/01647, filed on May 11, 2005.

(30) Foreign Application Priority Data
May 11, 2004 (JP) 232811/2004

(51) Int. Cl. A61K 48/00; C12Q 1/68; C07H 21/02

(52) U.S. Cl. 435/6; 514/44; 536/23.1

(57) ABSTRACT

The present invention provides a polynucleotide that not only has a high RNA interference effect on its target gene, but also has a very small risk of causing RNA interference against a gene unrelated to the target gene. A sequence segment conforming to the following rules (a) to (d) is searched from the base sequences of a target gene for RNA interference and, based on the search results, a polynucleotide capable of causing RNAi is designed, synthesized, etc.:
(a) The 3′ end base is adenine, thymine, or uracil,
(b) The 5′ end base is guanine or cytosine,
(c) A 7-base sequence from the 3′ end is rich in one or more types of bases selected from the group consisting of adenine, thymine, and uracil, and
(d) The number of bases is within a range that allows RNA interference to occur without causing cytotoxicity.

### TECHNICAL FIELD

The present invention relates to polynucleotides for causing RNA interference. Hereinafter, RNA interference may also be referred to as “RNAi.”

### BACKGROUND ART

RNA interference is a phenomenon of gene destruction wherein double-stranded RNA comprising sense RNA and anti-sense RNA (hereinafter also referred to as “dsRNA”) homologous to a specific region of a gene to be functionally inhibited, destructs the target gene by causing interference in the homologous portion of mRNA which is a transcript of the target gene. RNA interference was first proposed in 1998 following an experiment using nematodes. However, in mammals, when long dsRNA with about 30 or more base pairs is introduced into cells, an interferon response is induced, and cell death occurs due to apoptosis. Therefore, it was difficult to apply the RNAi method to mammals.

On the other hand, it was demonstrated that RNA interference could occur in early stage mouse embryos and cultured mammalian cells, and it was found that the induction mechanism of RNA interference also existed in the mammalian cells. At present, it has been demonstrated that short double-stranded RNA with about 21 to 23 base pairs (short interfering RNA, siRNA) can induce RNA interference without exhibiting cytotoxicity even in the mammalian cell system, and it has become possible to apply the RNAi method to mammals.

### DISCLOSURE OF THE INVENTION

The RNAi method is a technique which is expected to have various applications. However, while dsRNA or siRNA that is homologous to a specific region of a gene, exhibits an RNA interference effect in most of the sequences in drosophila and nematodes, 70% to 80% of randomly selected (21 base) siRNA do not exhibit an RNA interference effect in mammals. This poses a great problem when gene functional analysis is carried out using the RNAi method in mammals.

Conventional designing of siRNA has greatly depended on the experiences and sensory perceptions of the researcher or the like, and it has been difficult to design siRNA actually exhibiting an RNA interference effect with high probability. Other factors that prevent further research being conducted on RNA interference and its various applications are high costs and time consuming procedures required for carrying out an RNA synthesis resulting in part from the unwanted synthesis of siRNA.

In order to solve the above problems, the present invention aims to provide a polynucleotide capable of effectively acting as siRNA, a method for designing the same, a method for inhibiting gene expression using such a polynucleotide, a pharmaceutical composition comprising such a polynucleotide, and a composition for inhibiting gene expression.

### BRIEF DESCRIPTION OF THE DRAWINGS

FIG. 1 is a diagram which shows the designing of siRNA corresponding to sequences common to human and mice.

FIG. 2 is a diagram which shows the regularity of siRNA exhibiting an RNAi effect.

FIG. 3 is a diagram which shows common segments (shown in bold letters) having prescribed sequences in the base sequences of human FBP1 and mouse Fbp1.

FIG. 4 is a diagram listing prescribed sequences common to human FBP1 and mouse Fbp1.

FIG. 5 is a diagram in which the prescribed sequences common to human FBP1 and mouse Fbp1 are scored.

FIG. 6 is a diagram showing the results of BLAST searches on one of the prescribed sequences performed so that genes other than the target are not knocked out.

FIG. 7 is a diagram showing the results of BLAST searches on one of the prescribed sequences performed so that genes other than the target are not knocked out.

FIG. 8 is a diagram showing an output result of a program.

FIG. 9 is a diagram which shows the designing of RNA fragments (a to p).

FIG. 10 is a diagram showing the results of testing whether siRNA a to p exhibited an RNAi effect, in which “B” shows the results in drosophila cultured cells, and “C” shows the results in human cultured cells.

FIG. 11 is a diagram showing the analysis results concerning the characteristics of sequences of siRNA a to p.

FIG. 12 is a principle diagram showing the basic principle of the present invention.

FIG. 13 is a block diagram which shows an example of the configuration of a base sequence processing apparatus 100 of the system to which the present invention is applied.

FIG. 14 is a diagram which shows an example of information stored in a target gene base sequence file 106a.

FIG. 15 is a diagram which shows an example of information stored in a partial base sequence file 106b.

FIG. 16 is a diagram which shows an example of information stored in a determination result file 106c.

FIG. 17 is a diagram which shows an example of information stored in a prescribed sequence file 106d.

FIG. 18 is a diagram which shows an example of information stored in a reference sequence database 106e.

FIG. 19 is a diagram which shows an example of information stored in a degree of identity or similarity file 106f.

FIG. 20 is a diagram which shows an example of information stored in an evaluation result file 106g.

FIG. 21 is a block diagram which shows an example of the structure of a partial base sequence creation part 102a of the system to which the present invention is applied.

FIG. 22 is a block diagram which shows an example of the structure of an unrelated gene target evaluation part 102h of the system to which the present invention is applied.

FIG. 23 is a flowchart which shows an example of the main processing of the system in the embodiment.

FIG. 24 is a flowchart which shows an example of the unrelated gene evaluation process of the system in the embodiment.

FIG. 25 is a diagram which shows the structure of a target expression vector pTREC.

FIG. 26 is a diagram which shows the results of PCR in which one of the primers in Example 2, 2. (2) is designed such that no intron is inserted.

FIG. 27 is a diagram which shows the results of PCR in which one of the primers in Example 2, 2. (2) is designed such that an intron is inserted.

FIG. 28 is a diagram which shows the sequence and structure of siRNA; siVIM35.

FIG. 29 is a diagram which shows the sequence and structure of siRNA; siVIM812.

FIG. 30 is a diagram which shows the sequence and structure of siRNA; siControl.

FIG. 31 is a diagram which shows the results of assay of RNAi activity of siVIM812 and siVIM35.

FIG. 32 is a diagram which shows RNAi activity of siControl, siVIM812 and siVIM35 against vimentin.

FIG. 33 is a diagram which shows the results of antibody staining.

FIG. 34 is a diagram which shows the assay results of RNAi activity of siRNA designed by the program against the luciferase gene.

FIG. 35 is a diagram which shows the assay results of RNAi activity of siRNA designed by the program against the sequences of SARS virus.

FIG. 36 is a diagram which shows the assay results of RNAi activity of siRNA designed by the program against other genes containing sequences with a small number of mismatches to the siRNA.

FIG. 37 is a diagram which shows the relationship between apoptosis-related genes and GO_ID in Gene Ontology.

FIG. 38 is a diagram which shows the relationship between phosphatase or phosphatase activity-related genes and GO_ID in Gene Ontology.

FIG. 39 is a diagram which shows the relationship between cell cycle-related genes and GO_ID in Gene Ontology.

FIG. 40 is a diagram which shows the relationship between receptor-related genes and GO_ID in Gene Ontology.

FIG. 41 is a diagram which shows the relationship between ion channel-encoding genes and GO_ID in Gene Ontology.

FIG. 42 is a diagram which shows the relationship between signal transduction system-related genes and GO_ID in Gene Ontology.

FIG. 43 is a diagram which shows the relationship between kinase or kinase activity-related genes and GO_ID in Gene Ontology.

FIG. 44 is a diagram which shows the relationship between transcription regulation-related genes and GO_ID in Gene Ontology.

FIG. 45 is a diagram which shows the relationship between G protein-coupled receptor (GPCR) or GPCR-related genes and GO_ID in Gene Ontology.

FIG. 46 is a list of target sequences to be targeted by the polynucleotides of the present invention, along with their genes, biological function categories, reported biological functions and related diseases.

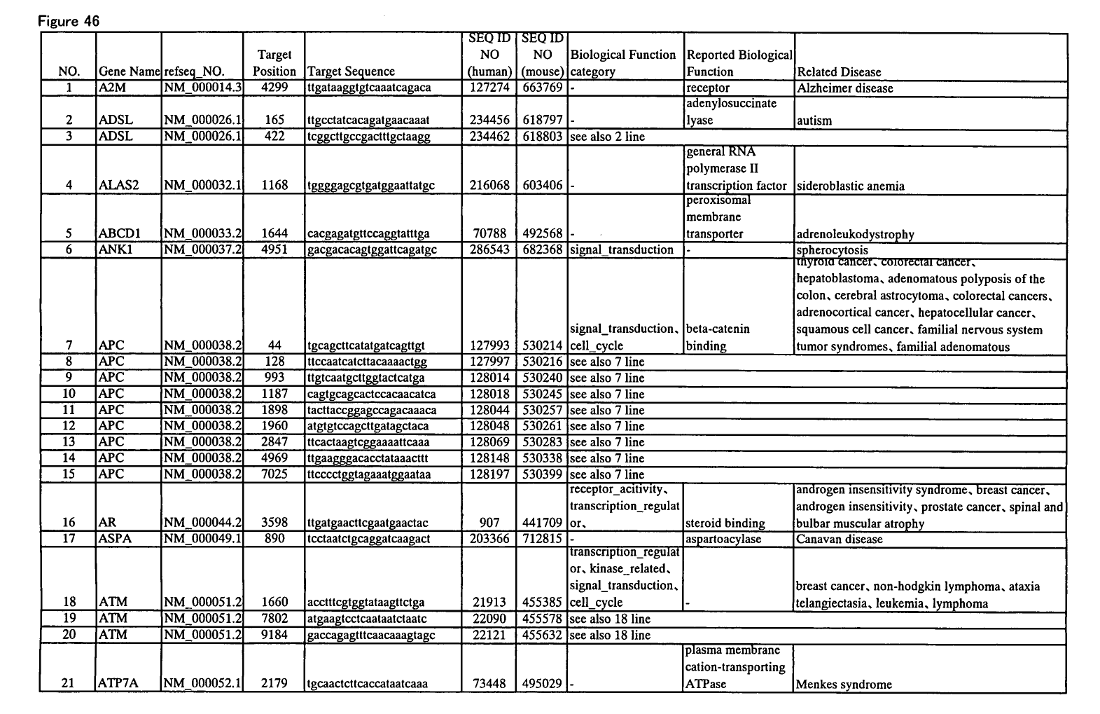

In FIG. 46, “Gene Name” and “refseq_NO.” correspond to the “RefSeq” database at NCBI (HYPERLINK “http://www.ncbi.nlm.nih.gov/” http://www.ncbi.nlm.nih.gov/), and information of each gene (including the sequence and function of the gene) can be obtained through access to the RefSeq database.

In FIG. 46, within the sequences listed in the column “Target Sequence,” actually targeted by RNAi is a portion covering the third base from the 5′ end to the third base from the 3′ end. Namely, the sequences listed in “Target Sequence” of FIG. 46 have, at both their 5′ and 3′ ends, additional 2 bases adjacent to the sequence targeted by RNAi. In the specification and claims of the present application, the term “prescribed sequence” in the narrow sense means a sequence actually targeted by RNAi and corresponds to, for example, a portion covering the third base from the 5′ end to the third base from the 3′ end of each “target sequence” in FIG. 46. For convenience sake, in the specification and claims of the present application, both terms “prescribed sequence” and “target sequence” are used, depending on the context, to mean the same sequence as the “prescribed sequence” in the narrow sense, or alternatively, to mean a sequence having additional 2 bases adjacent to the sequence targeted by RNAi, which are attached at both the 5′ and 3′ ends of the “prescribed sequence” in the narrow sense, as in the case of the “target sequences” in FIG. 46.

In the present specification and the claims, unless otherwise specified, the term “5′ end base” means the third base from the 5′ end of a sequence shown in the column “Target Sequence” of FIG. 46, while the term “3′ end base” means the third base from the 3′ end of a sequence shown in the column “Target Sequence” of FIG. 46. Thus, “Target Position” in FIG. 46 means a position in the sequence of each gene, which corresponds to the third base (for example, “g” in the case of the target sequence under Reference No. 1) from the 5′ end of each sequence shown in the column “Target Sequence” of FIG. 46.

In FIG. 46, “SEQ ID NO (human)” and “SEQ ID NO (mouse)” represent the sequence identification numbers (SEQ ID NOs) of individual target sequences shown in the sequence listing attached to this specification. Target sequences under the same reference number are identical between human and mouse.

### DETAILED DESCRIPTION OF THE INVENTION

In order to achieve the above object, the present inventors have studied a technique for easily obtaining siRNA, which is one of the steps requiring the greatest effort, time, and cost when the RNAi method is used. In view of the fact that preparation of siRNA is a problem especially in mammals, the present inventors have attempted to identify the sequence regularity of siRNA effective for RNA interference using mammalian cultured cell systems. As a result, it has been found that effective siRNA sequences have certain regularity, and thereby, the present invention has been completed. Namely, the present invention is as described below.

### [1] A polynucleotide for causing RNA interference against a target gene selected from the genes of a target organism, which has at least a double-stranded region,

wherein one strand in the double-stranded region consists of a base sequence homologous to a prescribed sequence which is contained in the base sequences of the target gene and which conforms to the following rules (a) to (d):

(a) The 3′ end base is adenine, thymine or uracil;
(b) The 5′ end base is guanine or cytosine;
(c) A 7-base sequence from the 3′ end is rich in one or more types of bases selected from the group consisting of adenine, thymine and uracil; and
(d) The number of bases is within a range that allows RNA interference to occur without causing cytotoxicity, and

wherein the other strand in the double-stranded region consists of a base sequence having a sequence complementary to the base sequence homologous to the prescribed sequence.

### [2] The polynucleotide according to [1], wherein at least 80% of bases in the base sequence homologous to the prescribed sequence corresponds to the base sequence of the prescribed sequence.

### [3] The polynucleotide according to [1] or [2], wherein, in the rule (c), at least three bases among the seven bases are one or more types of bases selected from the group consisting of adenine, thymine and uracil.

### [4] The polynucleotide according to any one of [1] to [3], wherein, in the rule (d), the number of bases is 13 to 28.

### [5] The polynucleotide according to any one of [1] to [4], wherein the prescribed sequence further conforms to the following rule (e):

(e) A sequence in which 10 or more bases of guanine or cytosine are continuously present is not contained.

### [6] The polynucleotide according to [5], wherein the prescribed sequence further conforms to the following rule (f):

(f) A sequence sharing at least 90% homology with the prescribed sequence is not contained in the base sequences of genes other than the target gene among all gene sequences of the target organism.

### [7] The polynucleotide according to [6], wherein the prescribed sequence consists of the base sequence shown in any of SEQ ID NOs: 47 to 817081.

### [8] The polynucleotide according to [6], wherein the prescribed sequence is any of the sequences listed in the column “Target Sequence” of FIG. 46.

### [9] The polynucleotide according to [6], which has any of the base sequences shown in SEQ ID NOs: 817102 to 817651.

### [10] The polynucleotide according to any one of [1] to [9], which is a double-stranded polynucleotide.

[11] The polynucleotide according to [10], wherein one strand of the double-stranded polynucleotide consists of a base sequence having an overhanging portion at the 3′ end of the base sequence homologous to the prescribed sequence, and the other strand of the double-stranded polynucleotide consists of a base sequence having an overhanging portion at the 3′ end of the sequence complementary to the base sequence homologous to the prescribed sequence.
[12] The polynucleotide according to any one of [1] to [9], which is a single-stranded polynucleotide having a hairpin structure, wherein the single-stranded polynucleotide has a loop segment linking the 3′ end of one strand in the double-stranded region and the 5′ end of the other strand in the double-stranded region.

### [13] A method for selecting a polynucleotide to be introduced into an expression system for a target gene whose expression is to be inhibited,

wherein the polynucleotide has at least a double-stranded region,

wherein one strand in the double-stranded region consists of a base sequence homologous to a prescribed sequence which is contained in the base sequences of the target gene and which conforms to the following rules (a) to (f):

(a) The 3′ end base is adenine, thymine or uracil;
(b) The 5′ end base is guanine or cytosine;
(c) A 7-base sequence from the 3′ end is rich in one or more types of bases selected from the group consisting of adenine, thymine and uracil;
(d) The number of bases is within a range that allows RNA interference to occur without causing cytotoxicity;
(e) A sequence in which 10 or more bases of guanine or cytosine are continuously present is not contained; and
(f) A sequence sharing at least 90% homology with the prescribed sequence is not contained in the base sequences of genes other than the target gene among all gene sequences of the target organism, and

wherein the other strand in the double-stranded region consists of a base sequence having a sequence complementary to the base sequence homologous to the prescribed sequence.

[14] The method for selecting a polynucleotide according to [13], wherein a polynucleotide having a sequence wherein the base sequence homologous to the prescribed sequence of the target gene contains mismatches of at least 3 bases against the base sequences of genes other than the target gene, and for which there is only a minimum number of other genes having a base sequence containing the mismatches of at least 3 bases, is further selected from the selected polynucleotides.
[15] A method for inhibiting gene expression, which comprises introducing the polynucleotide according to any one of [1] to [12] into an expression system for a target gene whose expression is to be inhibited, thereby inhibiting the expression of the target gene.
[16] A method for inhibiting gene expression, which comprises introducing a polynucleotide selected by the method according to [13] or [14] into an expression system for a target gene whose expression is to be inhibited, thereby inhibiting the expression of the target gene.

### [17] The method for inhibiting gene expression according to [15] or [16], wherein the expression is inhibited to 50% or below.

### [18] A pharmaceutical composition which comprises a pharmaceutically effective amount of the polynucleotide according to any one of [1] to [12].

### [19] The pharmaceutical composition according to [18], which is for use in treating or preventing the diseases listed in the column “Related Disease” of FIG. 46.

### [20] The pharmaceutical composition according to [18], which is for use in treating or preventing diseases related to the genes listed in the column “Gene Name” of FIG. 46.

[21] The pharmaceutical composition according to [18], which is for use in treating or preventing a disease in which a gene belonging to any of the following 1) to 9) is involved:
1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or

### 9) G protein-coupled receptor or a G protein-coupled receptor-related gene.

### [22] The pharmaceutical composition according to any one of [18] to [21], which comprises a polynucleotide targeting the base sequence shown in any of SEQ ID NOs listed in the column “SEQ ID NO (human)” or “SEQ ID NO (mouse)” of FIG. 46.

### [23] The pharmaceutical composition according to [18], which is for use in treating or preventing diseases related to the genes listed in the column “Gene Name” of Table 1.

[24] The pharmaceutical composition according to [18] or [23], which is for use in treating or preventing any cancer selected from bladder cancer, breast cancer, colorectal cancer, gastric cancer, hepatoma, lung cancer, melanoma, ovarian cancer, pancreas cancer, prostate cancer, oral cancer, skin cancer, and thyroid gland cancer.

### [25] The pharmaceutical composition according to any one of [18], [23] or [24], which comprises a polynucleotide having any of the base sequences shown in SEQ ID NOs: 817102 to 817651.

### [26] A composition for inhibiting gene expression to inhibit the expression of a target gene, which comprises the polynucleotide according to any one of [1] to [12].

### [27] The composition for inhibiting gene expression according to [26], wherein the target gene is related to any of the diseases listed in the column “Related Disease” of FIG. 46.

### [28] The composition for inhibiting gene expression according to [26], wherein the target gene is any of the genes listed in the column “Gene Name” of FIG. 46.

### [29] The composition for inhibiting gene expression according to [26], wherein the target gene is a gene belonging to any of the following 1) to 9):

1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or

### 9) G protein-coupled receptor or a G protein-coupled receptor-related gene.

### [30] The composition for inhibiting gene expression according to [26], wherein the target gene is any of the genes listed in the column “Gene Name” of Table 1.

[31] The composition for inhibiting gene expression according to [26], wherein the target gene is related to any cancer selected from bladder cancer, breast cancer, colorectal cancer, gastric cancer, hepatoma, lung cancer, melanoma, ovarian cancer, pancreas cancer, prostate cancer, oral cancer, skin cancer, and thyroid gland cancer.

### [32] A method for treating or preventing the diseases listed in the column “Related Disease” of FIG. 46, which comprises administering a pharmaceutically effective amount of the polynucleotide according to any one of [1] to [12].

### ADVANTAGES OF THE INVENTION

The polynucleotide of the present invention not only has a high RNA interference effect on its target gene, but also has a very small risk of causing RNA interference against a gene unrelated to the target gene, so that the polynucleotide of the present invention can cause RNA interference specifically only to the target gene whose expression is to be inhibited. Thus, the polynucleotide of the present invention is preferred for use in, e.g., tests and therapies using RNA interference, and is particularly effective in performing RNA interference in higher animals such as mammals, especially humans.

### EMBODIMENTS FOR CARRYING OUT THE INVENTION

The embodiments of the present invention will be described below in the order of the columns <1> to <7>.

### <1> Method for searching target base sequence of RNA interference

### <2> Method for designing base sequence of polynucleotide for causing RNA interference

### <3> Method for producing polynucleotide

### <4> Method for inhibiting gene expression

<5> siRNA sequence design program
<6> siRNA sequence design business model system

### <7> Base sequence processing apparatus for running siRNA sequence design program, etc.

### <8> Pharmaceutical composition

### <9> Composition for inhibiting gene expression

### <10> Method for treating or preventing diseases

### <1> Method for Searching Target Base Sequence of RNA Interference

The search method of the present invention is a method for searching a base sequence, which causes RNA interference, from the base sequences of a target gene selected from the genes of a target organism. The target organism, to which RNA interference is to be caused, is not particularly limited and may be a microorganism such as a prokaryotic organism (including E. coli), yeast or a fungus, an animal (including a mammal), an insect, a plant or the like.

Specifically, in the search method of the present invention, a sequence segment conforming to the following rules (a) to (d) is searched from the base sequences of a target gene for RNA interference.

(a) The 3′ end base is adenine, thymine or uracil.
(b) The 5′ end base is guanine or cytosine.
(c) A 7-base sequence from the 3′ end is rich in one or more types of bases selected from the group consisting of adenine, thymine and uracil.
(d) The number of bases is within a range that allows RNA interference to occur without causing cytotoxicity.

The term “gene” in the term “target gene” means a medium which codes for genetic information. The “gene” consists of a substance, such as DNA, RNA, or a complex of DNA and RNA, which codes for genetic information. As the genetic information, instead of the substance itself, electronic data of base sequences can be handled in a computer or the like. The “target gene” may be set as one coding region, a plurality of coding regions, or all the polynucleotides whose sequences have been revealed. When a gene with a particular function is desired to be searched, by setting only the particular gene as the target, it is possible to efficiently search the base sequences which cause RNA interference specifically in the particular gene. Namely, RNA interference is known as a phenomenon which destructs mRNA by interference, and by selecting a particular coding region, search load can be reduced. Moreover, a group of transcription regions may be treated as the target region to be searched. Additionally, in the present specification, base sequences are shown on the basis of sense strands, i.e., sequences of mRNA, unless otherwise described. Furthermore, in the present specification, a base sequence which satisfies the rules (a) to (d) is referred to as a “prescribed sequence”. In the rules, thymine corresponds to a DNA base sequence, and uracil corresponds to an RNA base sequence.

The rule (c) regulates so that a sequence in the vicinity of the 3′ end contains a rich amount of type(s) of base(s) selected from the group consisting of adenine, thymine, and uracil, and more specifically, as an index for search, regulates so that a 7-base sequence from the 3′ end is rich in one or more types of bases selected from adenine, thymine, and uracil.

In the rule (c), the phrase “sequence rich in” means that the frequency of a given base appearing is high, and schematically, a 5 to 10-base sequence, preferably a 7-base sequence, from the 3′ end in the prescribed sequence contains one or more types of bases selected from adenine, thymine, and uracil in an amount of preferably at least 40% or more, and more preferably at least 50%. More specifically, for example, in a prescribed sequence of about 19 bases, among 7 bases from the 3′ end, preferably at least 3 bases, more preferably at least 4 bases, and particularly preferably at least 5 bases, are one or more types of bases selected from the group consisting of adenine, thymine, and uracil.

The means for confirming the correspondence to the rule (c) is not particularly limited as long as it can be confirmed that preferably at least 3 bases, more preferably at least 4 bases, and particularly preferably at least 5 bases, among 7 bases are adenine, thymine, or uracil. For example, a case, wherein inclusion of 3 or more bases which correspond to one or more types of bases selected from the group consisting of adenine, thymine, and uracil in a 7-base sequence from the 3′ end is defined as being rich, will be described below. Whether the base is any one of the three types of bases is checked from the first base at the 3′ end one after another, and when three corresponding bases appear by the seventh base, conformation to the rule (c) is determined. For example, if three corresponding bases appear by the third base, checking of three bases is sufficient. That is, in the search with respect to the rule (c), it is not always necessary to check all of the seven bases at the 3′ end. Conversely, non-appearance of three or more corresponding bases by the seventh base means being not rich, thus being determined that the rule (c) is not satisfied.

In a double-stranded polynucleotide, it is well-known that adenine complementarily forms hydrogen-bonds to thymine or uracil. In the complementary hydrogen bond between guanine and cytosine (G-C hydrogen bond), three hydrogen bonding sites are formed. On the other hand, the complementary hydrogen bond between adenine and thymine or uracil (A-(T/U) hydrogen bond) includes two hydrogen bonding sites. Generally speaking, the bonding strength of the A-(T/U) hydrogen bond is weaker than that of the G-C hydrogen bond.

In the rule (d), the number of bases of the base sequence to be searched is regulated. The number of bases of the base sequence to be searched corresponds to the number of bases capable of causing RNA interference. Depending on the conditions, for example the species of an organism, in cases of siRNA having an excessively large number of bases, cytotoxicity is known to occur. The upper limit of the number of bases varies depending on the species of organism to which RNA interference is desired to be caused. The number of bases of the single strand constituting siRNA is preferably 30 or less regardless of the species. Furthermore, in mammals, the number of bases is preferably 24 or less, and more preferably 22 or less. The lower limit, which is not particularly limited as long as RNA interference is caused, is preferably at least 15, more preferably at least 18, and still more preferably at least 20. With respect to the number of bases as a single strand constituting siRNA, searching with a number of 21 is particularly preferable.

Furthermore, although a description will be made below, in siRNA, an overhanging portion is provided at the 3′ end of the prescribed sequence. The number of bases in the overhanging portion is preferably 2. Consequently, the upper limit of the number of bases in the prescribed sequence only, excluding the overhanging portion, is preferably 28 or less, more preferably 22 or less, and still more preferably 20 or less, and the lower limit is preferably at least 13, more preferably at least 16, and still more preferably at least 18. In the prescribed sequence, the most preferable number of bases is 19. The target base sequence for RNAi may be searched either including or excluding the overhanging portion.

Base sequences conforming to the prescribed sequence have an extremely high probability of causing RNA interference. Consequently, in accordance with the search method of the present invention, it is possible to search sequences that cause RNA interference with extremely high probability, and designing of polynucleotides which cause RNA interference can be simplified.

In another preferred example, the prescribed sequence may be a sequence further conforming to the following rule (e). (e) A sequence in which 10 or more bases of guanine or cytosine are continuously present is not contained.

The rule (e) regulates so that the base sequence to be searched does not contain a sequence in which 10 or more bases of guanine (G) and/or cytosine (C) are continuously present. Examples of the sequence in which 10 or more bases of guanine and/or cytosine are continuously present include a sequence in which either guanine or cytosine is continuously present as well as a sequence in which a mixed sequence of guanine and cytosine is present. More specific examples include GGGGGGGGGG, CCCCCCCCCC, and a mixed sequence of GCGGCCCGCG.

In order to prevent RNA interference from occurring in genes not related to the target gene, preferably, a search is made to determine whether a sequence that is identical or similar to the designed sequence is included in the other genes. A search for the sequence that is identical or similar to the designed sequence may be performed using software capable of performing a general homology search, etc. In this case, in consideration of the RNAi effect caused by two strands (sense and antisense strands) of siRNA, a search is more preferably made on both the “designed sequence” and a “sequence having a base sequence complementary to the designed sequence (complementary sequence)” to determine whether an identical or similar sequence is included in the other genes. When sequences having a sequence that is identical/similar to the designed sequence or its complementary sequence are excluded from the designed sequences, it is possible to design a sequence which causes RNA interference specifically to the target gene only.

Thus, when sequences for which other genes have similar sequences containing a small number of mismatches in their base sequences are excluded from the designed sequences, it is possible to select a sequence with high specificity. For example, in the case of designing a base sequence of 19 bases, it is preferable to exclude sequences for which other genes have similar sequences containing mismatches of 2 or less bases. In this case, if the number of mismatches, a threshold for similarity determination, is set at a higher value, a sequence to be designed will have a higher specificity. In the case of designing a base sequence of 19 bases, it is more preferable to exclude sequences for which other genes have similar sequences containing mismatches of 3 or less bases, and it is still more preferable to exclude sequences for which other genes have similar sequences containing mismatches of 4 or less bases. Moreover, when sequences for which other genes have similar sequences containing a small number of mismatches in their base sequences are excluded with respect to both a sequence having the prescribed sequence and its complementary sequence, such exclusion is preferred because it is possible to design a sequence with a higher specificity.

The number of mismatches, a criterion for determining sequence similarity, will also vary depending on the number of bases in a sequence to be designed, and is therefore difficult to define sweepingly. Given that the number of mismatches in a base sequence is defined by homology, a search may be made to determine whether the base sequence conforms to the following rule (f). (f) A sequence sharing at least 90% homology with the prescribed sequence is not contained in the base sequences of genes other than the target gene among all gene sequences of the target organism.

In the rule (f), the base sequences of genes other than the target gene preferably do not contain a sequence sharing at least 85% homology with the prescribed sequence, more preferably do not contain a sequence sharing at least 80% homology with the prescribed sequence, and still more preferably do not contain a sequence sharing at least 75% homology with the prescribed sequence. Moreover, when sequences for which other genes have similar sequences with high base sequence homology are excluded with respect to both a sequence having the prescribed sequence and its complementary sequence, such exclusion is preferred because it is possible to design a sequence with a higher specificity.

Furthermore, in the search of the prescribed sequence, detection can be efficiently performed by using a computer installed with a program which allows a search of segments conforming to the rules (a) to (c), etc., after determining the number of bases. More specific embodiments will be described below in the columns <5> siRNA sequence design program and <7> Base sequence processing apparatus for running siRNA sequence design program.

The polynucleotides shown in the sequence listing of the present application under SEQ ID NOs: 47 to 817081 are human and mouse sequences that are selected as prescribed sequences conforming to the above rules (a) to (f) or that are selected as target sequences containing the prescribed sequences.

### <2> Method for Designing Base Sequence of Polynucleotide for Causing RNA Interference

In the method for designing a base sequence in accordance with the present invention, a base sequence of polynucleotide which causes RNA interference is designed on the basis of the base sequence searched by the search method described above. A polynucleotide for causing RNA interference is a polynucleotide having a double-stranded region designed on the basis of the prescribed sequence searched by the above search method. Such a polynucleotide is not particularly limited as long as it can cause RNA interference against a target gene.

Polynucleotides for causing RNA interference may be principally classified into a double-stranded type (e.g., siRNA) and a single-stranded type (e.g., RNA with a hairpin structure (short hairpin RNA: shRNA)).

Although siRNA and shRNA are mainly composed of RNA, they also include hybrid polynucleotides partially containing DNA. In the method for designing a base sequence in accordance with the present invention, a base sequence conforming to the rules (a) to (d) is searched from the base sequences of a target gene, and a base sequence homologous to the searched base sequence is designed. In another preferred design example, it may be possible to take into consideration the above rules (e) and (f), etc. The rules (a) to (d) and the search method are the same as those described above regarding the search method of the present invention.

With respect to the double-stranded region in the polynucleotide for causing RNA interference, one strand consists of a base sequence homologous to a prescribed sequence which is contained in the base sequences of a target gene and which conforms to the above rules (a) to (d), and the other strand consists of a base sequence having a sequence complementary to the base sequence homologous to the prescribed sequence. The term “homologous sequence” refers to the same sequence and a sequence in which mutations, such as deletions, substitutions, and additions, have occurred to the same sequence to an extent that the function of causing the RNA interference has not been lost. Although depending on the conditions, such as the type and sequence of the target gene, the range of the allowable mutation, in terms of homology, is preferably 80% or more, more preferably 90% or more, and still more preferably 95% or more. When homology in the range of the allowable mutation is calculated, desirably, the numerical values calculated using the same search algorithm are compared. The search algorithm is not particularly limited. A search algorithm suitable for searching for local sequences is preferable. More specifically, BLAST, ssearch, or the like is preferably used.

More specifically, the percent identity between nucleic acids (polynucleotides) can be determined by visual inspection and mathematical calculation. Alternatively, the percent identity of two nucleic acid sequences can be determined by visual inspection and mathematical calculation, or more preferably, the comparison is done by comparing sequence information using a computer program. An exemplary, preferred computer program is the Genetic Computer Group (GCG; Madison, Wis.) Wisconsin package version 10.0 program, “GAP” (Devereux et al., 1984, Nucl. Acids Res. 12:387). In addition to making a comparison between two nucleic acid sequences, this “GAP” program can be used for comparison between two amino acid sequences and between a nucleic acid sequence and an amino acid sequence. The preferred default parameters for the “GAP” program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.2.7, available for use via the National Library of Medicine website: http://www.ncbi.nlm.nih.gov/blast/bl2seq/bls.html, or the UW-BLAST 2.0 algorithm. Standard default parameter settings for UW-BLAST 2.0 are described at the following Internet site: http://blast.wustl.edu. In addition, the BLAST algorithm uses the BLOSUM62 amino acid scoring matrix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul, 1990; if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The polynucleotide of the present invention also includes a polynucleotide that is hybridizable, as a “base sequence homologous” to a prescribed sequence conforming to the above rules (a) to (d), to the prescribed sequence under stringent conditions (e.g., under moderately or highly stringent conditions) and that preferably has the ability to cause RNA interference.

The term “under stringent condition” means that two sequences can hybridize under moderately or highly stringent conditions. More specifically, moderately stringent conditions can be readily determined by those having ordinary skill in the art, e.g., depending on the length of DNA. The basic parameters affecting the choice of hybridization conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of about 60° C., 0.5×SSC, 0.1% SDS. Preferably, moderately stringent conditions may include hybridization at about 50° C. and 6×SSC. Highly stringent conditions can also be readily determined by those skilled in the art, e.g., depending on the length of DNA. Generally, such conditions include hybridization and/or washing at higher temperature and/or lower salt concentration (such as hybridization at about 65° C., 6×SCC-0.2×SSC, preferably 6×SCC, more preferably 2×SSC, most preferably 0.2×SSC), compared to the moderately stringent conditions. For example, highly stringent conditions may include hybridization as defined above, and washing at approximately 68° C., 0.2×SSC, 0.1% SDS. SSPE (1×SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and wash buffers; washes are performed for 15 minutes after hybridization is completed.

It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 2001). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5° C. to 25° C. less than the melting temperature (Tm) of the hybrid, where Tm is determined according to the following equations. For hybrids less than 18 base pairs in length, Tm (° C.)=2(number of A+T bases)+4(number of G+C bases). For hybrids above 18 base pairs in length, Tm (° C.)=81.5° C.+16.6(log₁₀[Na⁺])+0.41(molar fraction [G+C])−0.63(% formamide−(500/N), where N is the number of bases in the hybrid, and [Na⁺] is the concentration of sodium ions in the hybridization buffer ([Na⁺] for 1×SSC=0.165 M).

As described above, although slight modification of the searched sequence is allowable, it is particularly preferred that the number of bases in the base sequence to be designed be the same as that of the searched sequence. For example, with respect to the allowance for change under the same number of bases, the bases of the base sequence to be designed correspond to those of the sequence searched at a rate of preferably 80% or more, more preferably 90% or more, and particularly preferably 95% or more. For example, when a base sequence having 19 bases is designed, preferably 16 or more bases, more preferably 18 or more bases, correspond to those of the searched base sequence. Furthermore, when a sequence homologous to the searched base sequence is designed, desirably, the 3′ end base of the base sequence searched is the same as the 3′ end base of the base sequence designed, and also desirably, the 5′ end base of the base sequence searched is the same as the 5′ end base of the base sequenced designed.

An overhanging portion is usually provided on a siRNA molecule. The overhanging portion is a protrusion provided on the 3′ end of each strand in a double-stranded RNA molecule. Although depending on the species of organism, the number of bases in the overhanging portion is preferably 2. Basically, any base sequence is acceptable in the overhanging portion. In some cases, the same base sequence as that of the target gene to be searched, TT, UU, or the like may be preferably used. As described above, by providing the overhanging portion at the 3′ end of the prescribed sequence which has been designed so as to be homologous to the base sequence searched, a sense strand constituting siRNA is designed.

Alternatively, it may be possible to search the prescribed sequence with the overhanging portion being included from the start to perform designing. The preferred number of bases in the overhanging portion is 2. Consequently, for example, in order to design a single strand constituting siRNA including a prescribed sequence having 19 bases and an overhanging portion having 2 bases, as the number of bases of siRNA including the overhanging portion, a sequence of 21 bases is searched from the target gene. Furthermore, when a double-stranded state is searched, a sequence of 23 bases may be searched.

shRNA is a single-stranded polynucleotide in which the 3′ end of one strand in the double-stranded region and the 5′ end of the other strand in the double-stranded region are linked through a loop segment. shRNA may have a protrusion in a single-stranded state at the 5′ end of the one strand and/or at the 3′ end of the other strand. Such shRNA can be designed according to known procedures as found in WO01/49844.

In the method for designing a base sequence in accordance with the present invention, as described above, a given sequence is searched from a desired target gene. The target to which RNA interference is intended to be caused does not necessarily correspond to the origin of the target gene, and is also applicable to an analogous species, etc. For example, it is possible to design siRNA used for a second species that is analogous to a first species using a gene isolated from the first species as a target gene. Furthermore, it is possible to design siRNA that can be widely applied to mammals, for example, by searching a common sequence from two or more species of mammals and searching a prescribed sequence from the common sequence to perform designing. The reason for this is that it is highly probable that the sequence common to two or more mammals exists in other mammals.

In the design method of the present invention, RNA molecules that cause RNA interference can be easily designed with high probability. Although synthesis of RNA still requires effort, time, and cost, the design method of the present invention can greatly minimize them.

### <3> Method for Producing Polynucleotide

By the method for producing a polynucleotide in accordance with the present invention, a polynucleotide that has a high probability of causing RNA interference can be produced. For the polynucleotide of the present invention, a base sequence of the polynucleotide is designed in accordance with the method for designing the base sequence of the present invention described above, and a polynucleotide is synthesized so as to follow the sequence design. Although, as described above, the polynucleotide of the present invention includes both double-stranded type (e.g., siRNA) and single-stranded type (e.g., shRNA), the following explanation will be made principally for double-stranded polynucleotides.

Preferred embodiments in the sequence design are the same as those described above regarding the method for designing the base sequence. Additionally, the double-stranded polynucleotide produced by the production method of the present invention is preferably composed of RNA, but a hybrid polynucleotide which partially contains DNA may be acceptable. In this specification, double-stranded polynucleotides partially containing DNA are also included in the concept of siRNA. Also, RNA and DNA constituting the polynucleotide may have chemical modifications such as methylation of sugar hydroxyl groups. For example, siRNA in this specification may have a hybrid structure composed of a DNA strand and an RNA strand. Although such a hybrid structure is not particularly limited as long as it provides the ability to inhibit the expression of a target gene when introduced into a recipient, it is desired that such a hybrid polynucleotide is a double-stranded polynucleotide having a sense strand composed of DNA and an antisense strand composed of RNA.

Alternatively, siRNA in this specification may also have a chimeric structure. The chimeric structure refers to a structure containing both DNA and RNA in a single-stranded polynucleotide. Such a chimeric structure is not particularly limited as long as it provides the ability to inhibit the expression of a target gene when introduced into a recipient. According to the research conducted by the present inventors, siRNA tends to have structural and functional asymmetry, and in view of the object of causing RNA interference, a half of the sense strand at the 5′ end side and a half of the antisense strand at the 3′ end side are desirably composed of RNA.

Incidentally, in siRNA having a chimeric structure, the content of RNA is preferably minimized in terms of in vivo stability in a recipient and production costs, etc. To this end, the inventors have made extensive and intensive efforts to study siRNA whose RNA content can be reduced while maintaining a high inhibitory effect on the expression of a target gene. As a result, the inventors have obtained the results indicating that a portion of 9 to 13 nucleotides from the 5′ end of the sense strand and a portion of 9 to 13 nucleotides from the 3′ end of the antisense strand (e.g., portions of 11 nucleotides, preferably 10 nucleotides, more preferably 9 nucleotides, from the above respective ends of the sense and antisense strands) are desirably composed of RNA and, in particularly, the 3′ end side of the antisense strand desirably has such a structure. The positions of RNA portions in the sense and antisense strands are not necessarily matched.

In a double-stranded polynucleotide, one strand is formed by providing an overhanging portion to the 3′ end of a base sequence homologous to the prescribed sequence conforming to the rules (a) to (d) contained in the base sequence of the target gene, and the other strand is formed by providing an overhanging portion to the 3′ end of a base sequence complementary to the base sequence homologous to the prescribed sequence. The number of bases in each strand, including the overhanging portion, is 18 to 24, more preferably 20 to 22, and particularly preferably 21. The number of bases in the overhanging portion is preferably 2. siRNA having 21 bases in total in which the overhanging portion is composed of 2 bases is suitable for causing RNA interference with high probability without causing cytotoxicity even in mammals.

RNA may be synthesized, for example, by chemical synthesis or by standard biotechnology. In one technique, a DNA strand having a predetermined sequence is produced, single-stranded RNA is synthesized using the produced DNA strand as a template in the presence of a transcriptase, and the synthesized single-stranded RNA is formed into double-stranded RNA.

With respect to the basic technique for molecular biology, there are many standard, experimental manuals, for example, BASIC METHODS IN MOLECULAR BIOLOGY (1986); Sambrook et al., MOLECULAR CLONING; A LABORATORY MANUAL, Second Edition, Cold Spring Harbor Laboratory Press, Cold Spring Harbor, N.Y. (1989); Saibo-Kogaku Handbook (Handbook for cell engineering), edited by Toshio Kuroki et al., Yodosha (1992); and Shin-Idenshi-Kogaku Handbook (New handbook for genetic engineering), edited by Muramatsu et al., Yodosha (1999).

One preferred embodiment of polynucleotide produced by the production method of the present invention is a double-stranded polynucleotide produced by a method in which a sequence segment including 13 to 28 bases conforming to the rules (a) to (d) is searched from a base sequence of a target gene for RNA interference, one strand is formed by providing an overhanging portion at the 3′ end of a base sequence homologous to the prescribed sequence following the rules (a) to (d), the other strand is formed by providing an overhanging portion at the 3′ end of a sequence complementary to the base sequence homologous to the prescribed sequence, and synthesis is performed so that the number of bases in each strand is 15 to 30. The resulting polynucleotide has a high probability of causing RNA interference.

It is also possible to prepare an expression vector which expresses siRNA. By placing a vector which expresses a sequence containing the prescribed sequence under a condition of a cell line or cell-free system in which expression is allowed to occur, it is possible to supply predetermined siRNA using the expression vector.

Since conventional designing of siRNA has depended on the experiences and intuition of the researcher, trial and error have often been repeated. However, by the double-stranded polynucleotide production method in accordance with the present invention, it is possible to produce a double-stranded polynucleotide which causes RNA interference with high probability. In accordance with the search method, sequence design method, or polynucleotide production method of the present invention, it is possible to greatly reduce effort, time, and cost required for various experiments, manufacturing, etc., which use RNA interference. Namely, the present invention greatly simplifies various experiments, research, development, manufacturing, etc., in which RNA interference is used, such as gene analysis, search for targets for new drug development, development of new drugs, gene therapy, and research on differences between species, and thus efficiency can be improved.

In one embodiment, the present invention also provides a method for selecting the polypeptide of the present invention described above. More specifically, the present invention provides a method for selecting a polynucleotide to be introduced into an expression system for a target gene whose expression is to be inhibited,

wherein the polynucleotide has at least a double-stranded region,

wherein one strand in the double-stranded region consists of a base sequence homologous to a prescribed sequence which is contained in the base sequences of the target gene and which conforms to the following rules (a) to (f):

(a) The 3′ end base is adenine, thymine or uracil;
(b) The 5′ end base is guanine or cytosine;
(c) A 7-base sequence from the 3′ end is rich in one or more types of bases selected from the group consisting of adenine, thymine and uracil;
(d) The number of bases is within a range that allows RNA interference to occur without causing cytotoxicity;
(e) A sequence in which 10 or more bases of guanine or cytosine are continuously present is not contained; and
(f) A sequence sharing at least 90% homology with the prescribed sequence is not contained in the base sequences of genes other than the target gene among all gene sequences of the target organism, and

wherein the other strand in the double-stranded region consists of a base sequence having a sequence complementary to the base sequence homologous to the prescribed sequence.

The sequence to be targeted by the polypeptide obtained by the selection method of the present invention is a sequence selected as a prescribed sequence conforming to the above rules (a) to (f). Preferably, such a sequence may be any of SEQ ID NOs: 47 to 817081.

In the selection method of the present invention, a polynucleotide having a sequence, wherein the base sequence homologous to the prescribed sequence of the target gene contains mismatches of at least 3 bases against the base sequences of genes other than the target gene, and for which there is only a minimum number of other genes having a base sequence containing the mismatches of at least 3 bases, may further be selected from the selected polynucleotides.

Namely, if the target sequence is a sequence highly specific to the target gene, the polynucleotide selectively produces an inhibitory effect only on the expression of the target gene containing the target sequence, but not on the other genes (i.e., the polynucleotide has less off-target effect), thus reducing influences of side effects, etc. It is therefore more preferred that the target sequence of the polynucleotide has high specificity to the target gene. Among the selected sequences (e.g., SEQ ID NOs: 47 to 817081), a sequence whose off-target effect can be further reduced is preferred as a prescribed sequence conforming to the above rules (a) to (f). As a preferred prescribed sequence of the target gene, it is possible to select a sequence which contains mismatches of at least 3 bases against the base sequences of other genes and for which there is a minimum number of other genes having a base sequence containing mismatches of at least 3 bases. The requirement “there is only a minimum number of other genes” means that “other genes having a base sequence containing mismatches of at least 3 bases” (i.e., similar genes) are as few in number as possible; for example, there are preferably 10 or less genes, more preferably 6 or less genes, still more preferably only one gene, or most preferably no gene.

For example, the 53998 sequences shown in FIG. 46 are obtained among SEQ ID NOs: 47 to 817081 by selecting sequences which contain mismatches of 3 bases against the base sequences of other genes (i.e., prescribed sequences of 19 bases (in the narrow sense) in which 16 bases other than these 3 mismatched bases are the same as those of other genes) and for which there is only a minimum number of other genes having a base sequence containing mismatches of 3 bases. Thus, the target sequence is particularly preferably any of these sequences.

### <4> Method for Inhibiting Gene Expression

The method for inhibiting gene expression in accordance with the present invention includes a step of searching a predetermined base sequence, a step of designing and synthesizing a base sequence of a polynucleotide based on the searched base sequence, and a step of introducing the resulting polynucleotide into an expression system containing a target gene.

The step of searching a predetermined base sequence follows the method for searching a target base sequence for RNA interference described above. Preferred embodiments are the same as those described above. The step of designing and synthesizing the base sequence of siRNA based on the searched base sequence can be carried out in accordance with the method for designing the base sequence of a polynucleotide for causing RNA interference and the method for producing a polynucleotide described above. Preferred embodiments are the same as those described above.

The resulting polynucleotide is added to an expression system for a target gene to inhibit the expression of the target gene. The expression system for a target gene means a system in which the target gene is expressed, and more specifically, a system provided with a reaction system in which at least mRNA of the target gene is formed. Examples of the expression system for a target gene include both in vitro and in vivo systems. In addition to cultured cells, cultured tissues, and living bodies, cell-free systems can also be used as expression systems for target genes. The target gene whose expression is intended to be inhibited (inhibition target gene) is not necessarily a gene of a species corresponding to the origin of the searched sequence. However, as the relationship between the origin of the search target gene and the origin of the inhibition target gene becomes closer, a predetermined gene can be more specifically and effectively inhibited.

Introduction into an expression system for a target gene means incorporation into the expression reaction system for the target gene. For example, in one method, a double-stranded nucleotide is transfected to a cultured cell including a target gene and incorporated into the cell. In another method, an expression vector having a base sequence comprising a prescribed sequence and an overhanging portion is formed, and the expression vector is introduced into a cell having a target gene (WO01/36646, WO01/49844).

In accordance with the gene inhibition method of the present invention, since polynucleotides which cause RNA interference can be efficiently produced, it is possible to inhibit genes efficiently and simply. Thus, for example, in a case where the target gene is a disease-related gene, siRNA (or shRNA) targeting the disease-related gene or a vector expressing such siRNA (or shRNA) may be introduced into cells which express the disease-related gene, so that the disease-related gene can be made inactive.

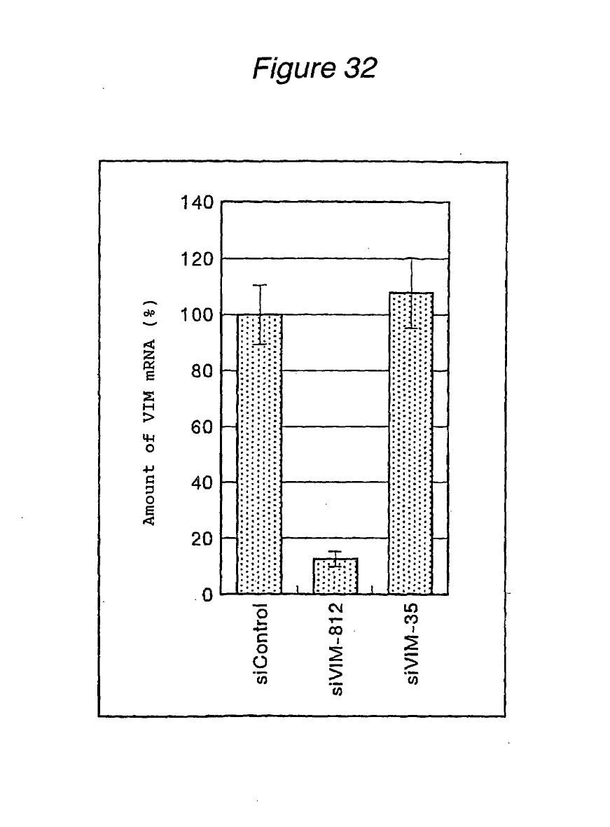

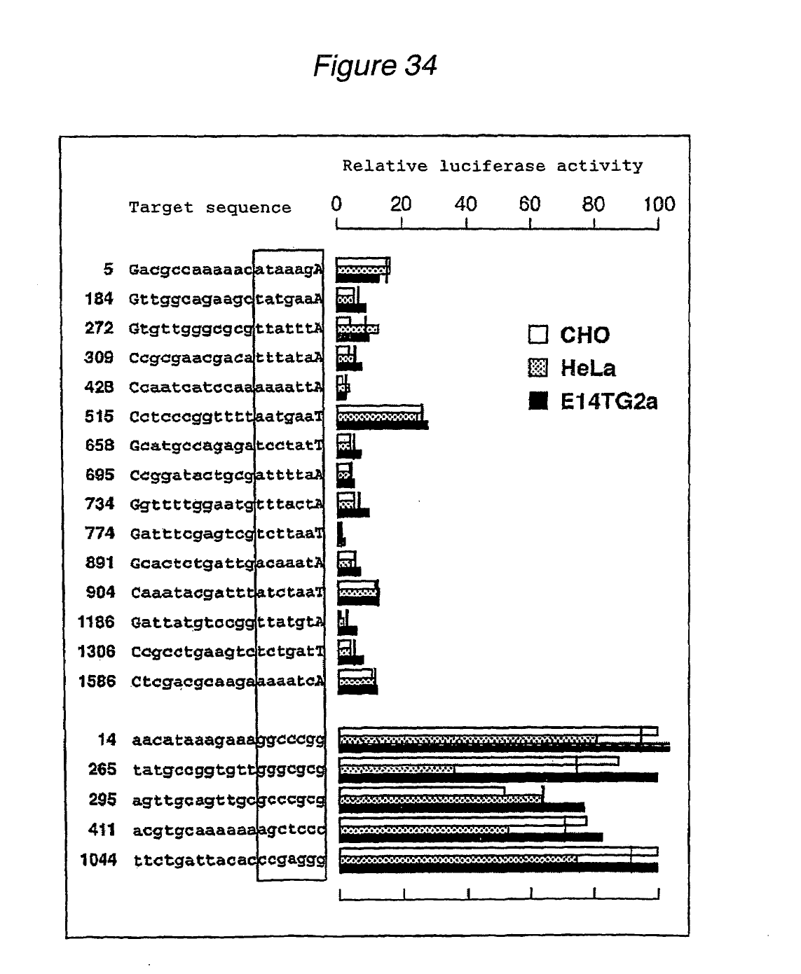

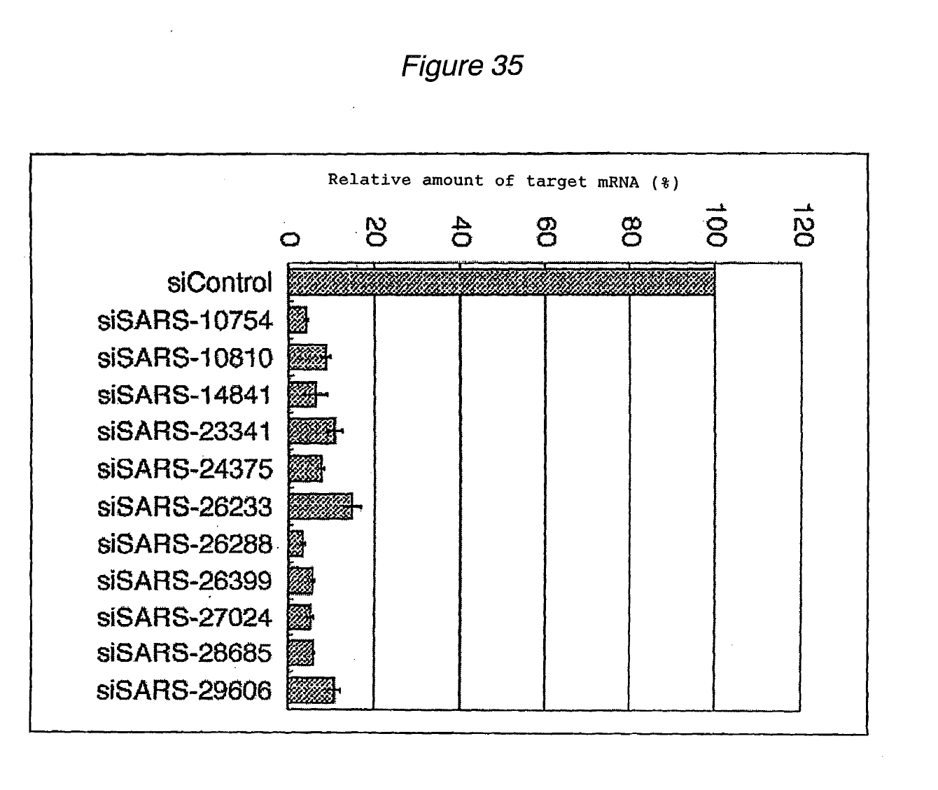

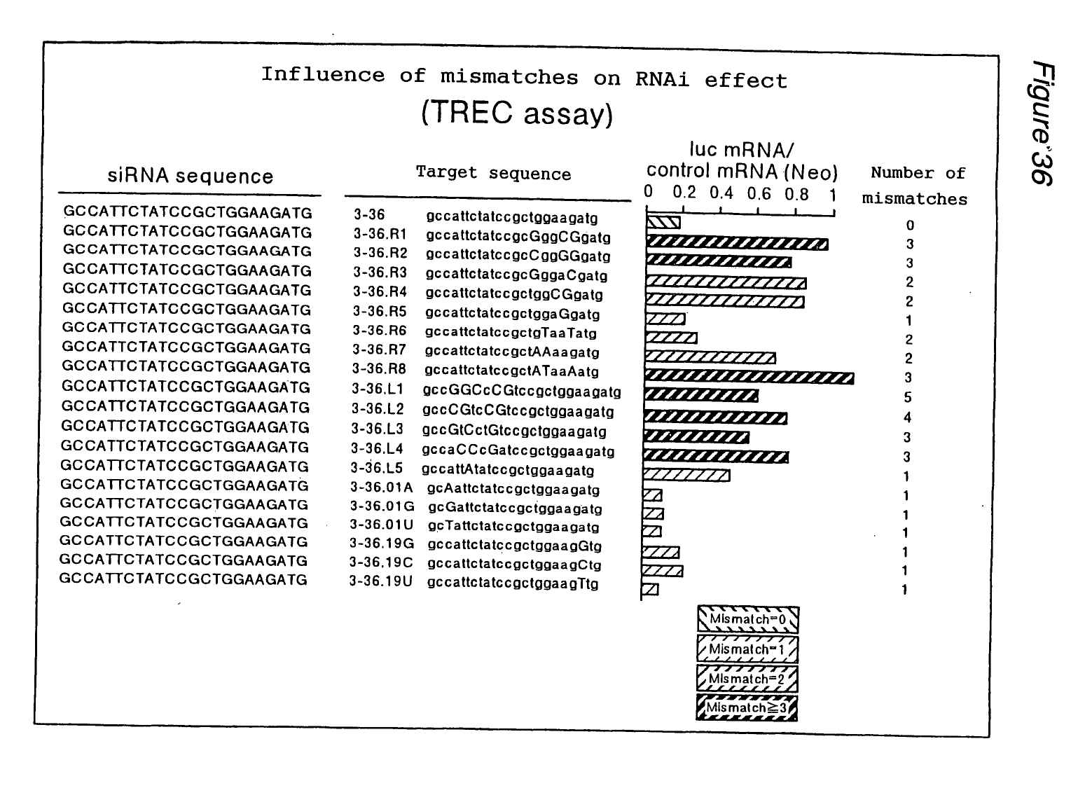

In Examples 2 to 5 described herein later, the RNAi effect of the polynucleotide of the present invention against the genes of human vimentin, luciferase, SARS virus and the like was examined as a relative expression level of mRNA compared to the control. FIGS. 31, 32 and 35 show the results of mRNA expression levels measured by quantitative PCR. In FIGS. 31, 32 and 35, the relative mRNA expression levels are respectively reduced to about 7-8% (Example 2, FIG. 31), about 12-13% (Example 3, FIG. 32), and a few % to less than about 15% (Example 5, FIG. 35); the polynucleotide of the present invention was confirmed to have an inhibitory effect on the expression of each gene. Likewise, FIG. 34 from Example 4 shows the results of mRNA expression levels (as RNAi effect) examined by luciferase activity. The luciferase activity was also reduced to a few % to less than about 20%, as compared to the control.

Moreover, in Example 8, among the genes shown in FIG. 46 whose related diseases and/or biological functions have been identified, about 300 genes selected at random were examined for the expression levels of their mRNA in human-derived HeLa cells, expressed as relative expression levels. As shown in Table 1, the RQ values (described later) that were calculated to evaluate an inhibitory effect on the expression of these genes, i.e., an RNAi effect were all less than 1, and almost all less than 0.5.

In the method for inhibiting gene expression in accordance with the present invention, the phrase “inhibiting the expression of the target gene” means that the mRNA expression level of the target gene is substantially reduced. If the mRNA expression level has been substantially reduced, inhibited expression has been achieved regardless of the degree of change in the mRNA expression level. In particular, since a larger amount of reduction means a higher inhibitory effect on expression, the criterion for inhibited expression may be, without being limited to, a case where the mRNA expression level is preferably reduced to about 80% or below, more preferably reduced to about 50% or below, still more preferably reduced to about 20% or below, still even more preferably reduced to about 15% or below, and further preferably reduced to about 8% or below. In accordance with the gene inhibition method of the present invention which uses a polynucleotide selected according to the rules of the present invention, it becomes possible to preferably cause at least a 50% or more reduction in the mRNA expression level of the target gene.

<5> siRNA Sequence Design Program

Embodiments of the siRNA sequence design program will be described below.

(5-1) Outline of the Program

When species whose genomes are not sequenced, for example, horse and swine, are subjected to RNA interference, this program calculates a sequence of siRNA usable in the target species based on published sequence information regarding human beings and mice. If siRNA is designed using this program, RNA interference can be carried out rapidly without sequencing the target gene. In the design (calculation) of siRNA, sequences having RNAi activity with high probability are selected in consideration of the rules of allocation of G or C (the rules (a) to (d) described above), and checking is performed by homology search so that RNA interference does not occur in genes that are not related to the target gene. In this specification, “G or C” may also be written as “G/C”, and “A or T” may also be written as “A/T”. Furthermore, “T(U)” in “A/T(U)” means T (thymine) in the case of sequences of deoxyribonucleic acid and U (uracil) in the case of sequences of ribonucleic acid.

(5-2) Policy of siRNA Design

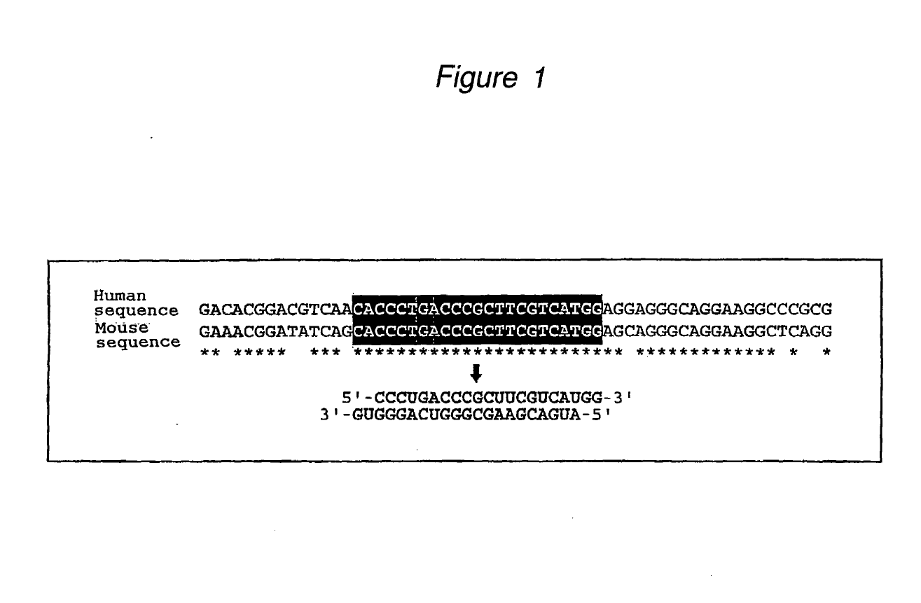

Sequences of human gene X and mouse gene X which are homologous to the human gene are assumed to be known. This program reads the sequences and searches completely common sequences each having 23 or more bases from the coding regions (CDS). By designing siRNA from the common portions, the resulting siRNA can target both human and mouse gene X (FIG. 1).

Since the portions completely common to human beings and mice are believed to also exist in other mammals with high probability, the siRNA is expected to act not only on gene X of human beings and mice but also on gene X of other mammals. Namely, even if in an animal species in which the sequence of a target gene is not known, if sequence information is known regarding the corresponding homologues of human beings and mice, it is possible to design siRNA using this program.

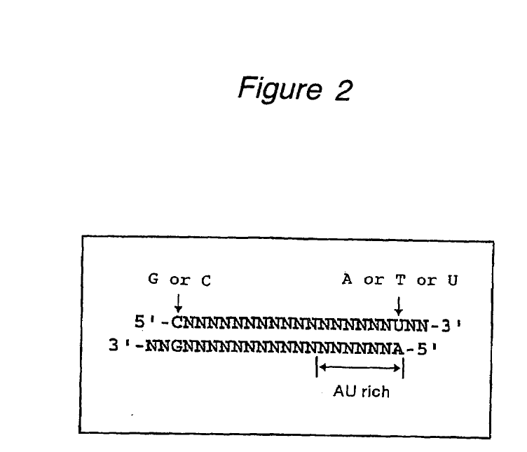

Furthermore, in mammals, it is known that sequences of effective siRNA have regularity (FIG. 2). In this program, only sequences conforming to the rules are selected. FIG. 2 is a diagram which shows regularity of siRNA sequences exhibiting an RNAi effect (rules of G/C allocation of siRNA). In FIG. 2, with respect to siRNA in which two RNA strands, each having a length of 21 bases and having an overhang of 2 bases on the 3′ side, form base pairs between 19 bases at the 5′ side of the two strands, the sequence in the coding side among the 19 bases forming the base pairs must satisfy the following conditions: 1) The 3′ end is A/U; 2) the 5′ end is G/C, and 3) 7 characters on the 3′ side has a high ratio of A/U. In particular, the conditions 1) and 2) are important.

(5-3) Structure of Program

This program consists of three parts, i.e., (5-3-1) a part which searches sequences of sites common to human beings and mice (partial sequences), (5-3-2) a part which scores the sequences according to the rules of G/C allocation, and (5-3-3) a part which performs checking by homology search so that unrelated genes are not targeted.

(5-3-1) Part which Searches Common Sequences

This part reads a plurality of base sequence files (file 1, file 2, file 3, . . . ) and finds all sequences of 23 characters that commonly appear in all the files.

### CALCULATION EXAMPLE

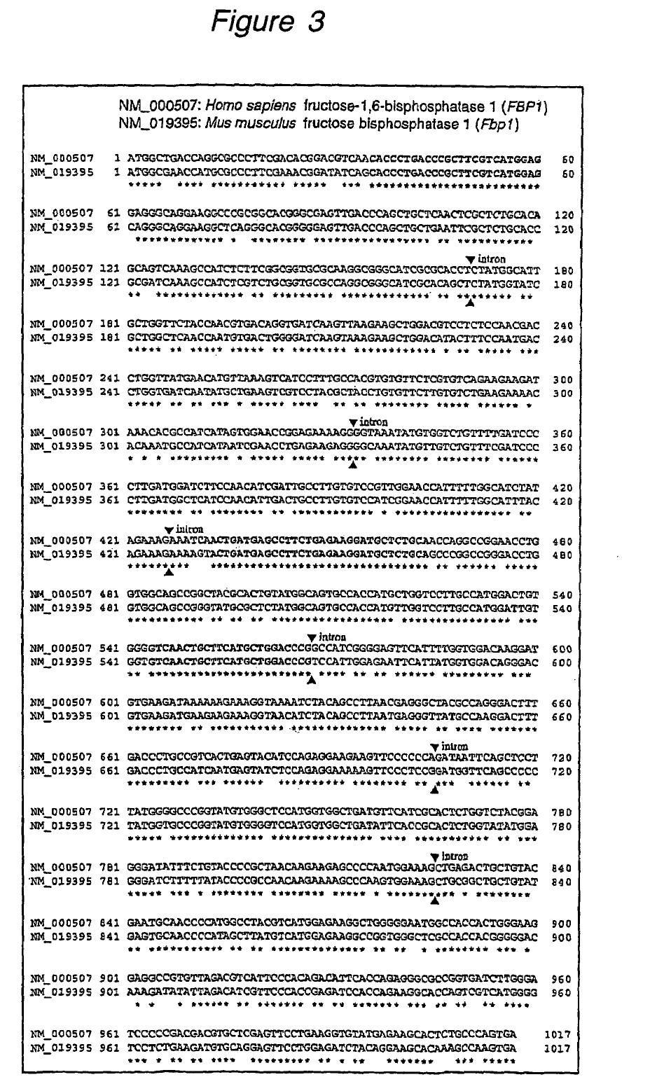

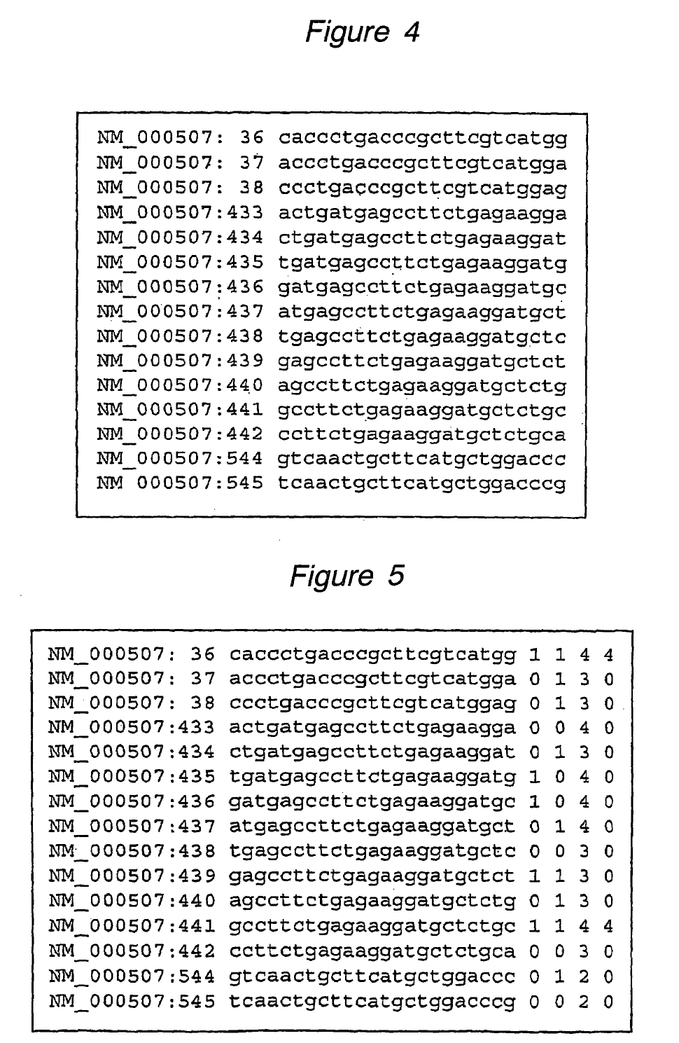

As file 1, sequences of human gene FBP1 (HM_{—}000507: Homo sapiens fructose-1,6-bisphosphatase 1) and, as file 2, sequences of mouse gene Fbp1 (NM_{—}019395: Mus musculus fructose bisphosphatase 1) were inputted into the program. As a result, from the sequences of the two (FIG. 3), 15 sequences, each having 23 characters, that were common to the two (sequences common to human FBP1 and mouse Fbp1) were found (FIG. 4).

(5-3-2) Part which Scores Sequences

This part scores the sequences each having 23 characters in order to only select the sequences conforming to the rules of G/C allocation.

(Method)

The sequences each having 23 characters are scored in the following manner.

Score 1: Is the 21st character from the head A/U?
[no=0, yes=1]

Score 2: Is the third character from the head G/C?
[no=0, yes=1]

Score 3: The number of A/U among 7 characters between the 15th character and 21st character from the head
[0 to 7]

Total score: Product of scores 1 to 3. However, if the product is 3 or less, the total score is considered as zero.

### CALCULATION EXAMPLE

With respect to 15 sequences in FIG. 4, the results of calculation are shown in FIG. 5. FIG. 5 is a diagram in which the sequences common to human FBP1 and mouse Fbp1 are scored. Furthermore, score 1, score 2, score 3, and total score are described in this order after the sequences shown in FIG. 5.

(5-3-3) Part which Performs Checking so that Unrelated Genes are Not Targeted

In order to prevent the designed siRNA from acting on genes unrelated to the target gene, homology search is performed against all the published mRNA of human beings and mice, and the degree of unrelated genes being hit is evaluated. Various search algorithms can be used in the homology search. Herein, an example in which BLAST is used will be described. Additionally, when BLAST is used, in view that the sequences to be searched are as short as 23 bases, it is desirable that Word Size be decreased sufficiently.

After the Blast search, among the hits with an E-value of 10.0 or less, with respect to all the hits other than the target gene, the total sum of the reciprocals of the E-values are calculated (hereinafter, the value is referred to as a homology score). Namely, the homology score (X) is found in accordance with the following expression.

$$X = {\sum }_{all hits} \frac{1}{E}$$

Note: A lower E value of the hit indicates higher homology to 23 characters of the query and higher risk of being targeted by siRNA. A larger number of hits indicates a higher probability that more unrelated genes are targeted. In consideration of these two respects, the risk that siRNA targets genes unrelated to the target gene is evaluated using the above expression.

### CALCULATION EXAMPLE

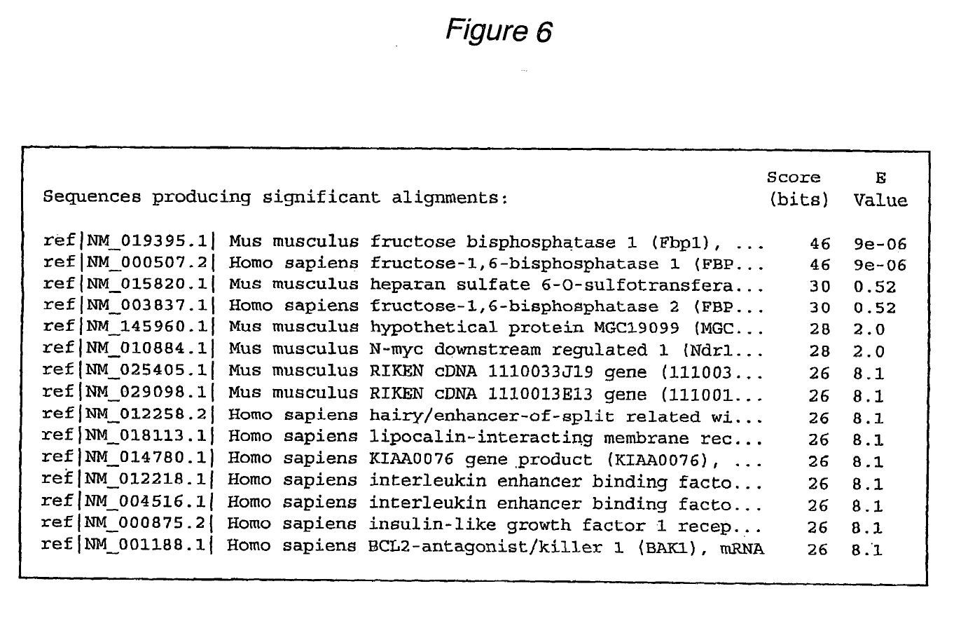

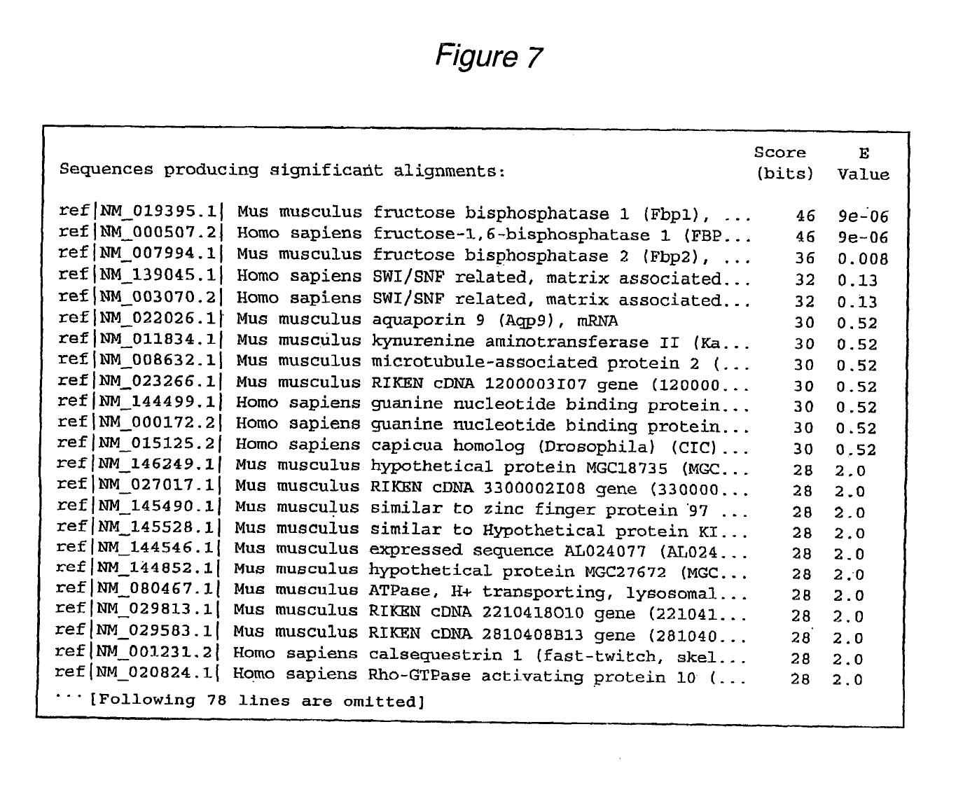

The results of homology search against the sequences each having 23 characters and the homology scores are shown (FIGS. 6 and 7). FIG. 6 shows the results of BLAST searches of a sequence common to human FBP1 and mouse Fbp1, i.e., “caccctgacccgcttcgtcatgg”, and the first two lines are the results in which both mouse Fbp1 and human FBP1 are hit. The homology score is 5.9, and this is an example of a small number of hits. The risk that siRNA of this sequence targets other genes is low. Furthermore, FIG. 7 shows the results of BLAST searches of a sequence common to human FBP1 and mouse Fbp1, i.e., “gccttctgagaaggatgctctgc”. This is an example of a large number of hits, and the homology score is 170.8. Since the risk of targeting other genes is high, the sequence is not suitable as siRNA.

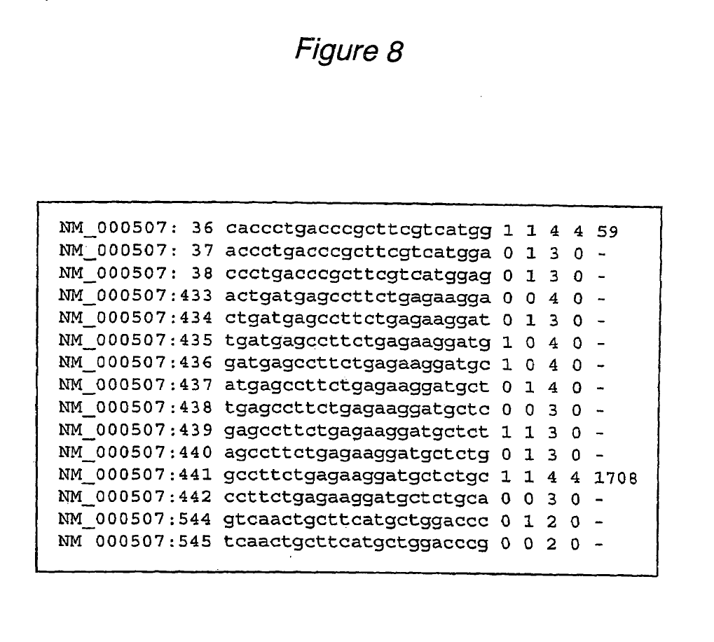

In practice, the parts (5-3-1), (5-3-2) and (5-3-3) may be integrated, and when the sequences of human beings and mice shown in FIG. 3 are inputted, an output as shown in FIG. 8 is directly obtained. Herein, after the sequences shown in FIG. 8, score 1, score 2, score 3, total score, and the tenfold value of homology score are described in this order. Additionally, in order to save processing time, the program may be designed so that the homology score is not calculated when the total score is zero. As a result, it is evident that the segment “36 caccctgacccgcttcgtcatgg” can be used as siRNA. Furthermore, one of the parts (5-3-1), (5-3-2) and (5-3-3) may be used independently.

(5-4) Actual Calculation

With respect to about 6,400 gene pairs among the homologues between human beings and mice, siRNA was actually designed using this program. As a result, regarding about 70% thereof, it was possible to design siRNA which had a sequence common to human beings and mice and which satisfied the rules of effective siRNA sequence regularity so that unrelated genes were not targeted.

These siRNA sequences are expected to effectively inhibit target genes not only in human beings and mice but also in a wide range of mammals, and are believed to have a high industrial value, such as applications to livestock and pet animals. Moreover, it is possible to design siRNA which simultaneously targets two or more genes of the same species, e.g., eIF2C1 and eIF2C2, using this program. Thus, the method for designing siRNA provided by this program has a wide range of application and is extremely strong. In further application, by designing a PCR primer using a sequence segment common to human beings and mice, target genes can be amplified in a wide range of mammals.

Additionally, embodiments of the apparatus which runs the siRNA sequence design program will be described in detail below in the column <7> Base sequence processing apparatus for running siRNA sequence design program.

<6> siRNA Sequence Design Business Model System

In the siRNA sequence design business model system of the present invention, when the siRNA sequence design program is applied, the system refers to a genome database, an EST database, and a phylogenetic tree database, alone or in combination, according to the logic of this program, and effective siRNA in response to availability of gene sequence information is proposed to the client. The term “availability” means a state in which information is available.

(1) In a case in which it is difficult to specify an ORF although genome information is available, siRNA candidates effective against assumed exon sites are extracted based on EST information, etc., and siRNA sequences in consideration of splicing variants and evaluation results thereof are displayed.

### (2) In a case in which a gene sequence and a gene name are known, after the input of the gene sequence or the gene name, effective siRNA candidates are extracted, and siRNA sequences and evaluation results thereof are displayed.

(3) In a case in which genome information is not available, using the gene sequences of a related species storing the same type of gene functions (congeneric or having the same origin) or gene sequences of two or more species which have a short distance in phylogenetic trees and of which genome sequences are available, effective siRNA candidates are extracted, and siRNA sequences and evaluation results thereof are displayed.
(4) In order to analyze functions of genes relating infectious diseases and search for targets for new drug development, a technique is effective in which the genome database and phylogenetic tree database of microorganisms are further combined with apoptosis induction site information and function expression site information of microorganisms to obtain exhaustive siRNA candidate sequences.

### <7> Base Sequence Processing Apparatus for Running siRNA Sequence Design Program, etc.

Embodiments of the base sequence processing apparatus which is an apparatus for running the siRNA sequence design program described above, the program for running a base sequence processing method on a computer, the recording medium, and the base sequence processing system in accordance with the present invention will be described in detail below with reference to the drawings. However, it is to be understood that the present invention is not restricted by the embodiments.

### [Summary of the Present Invention]

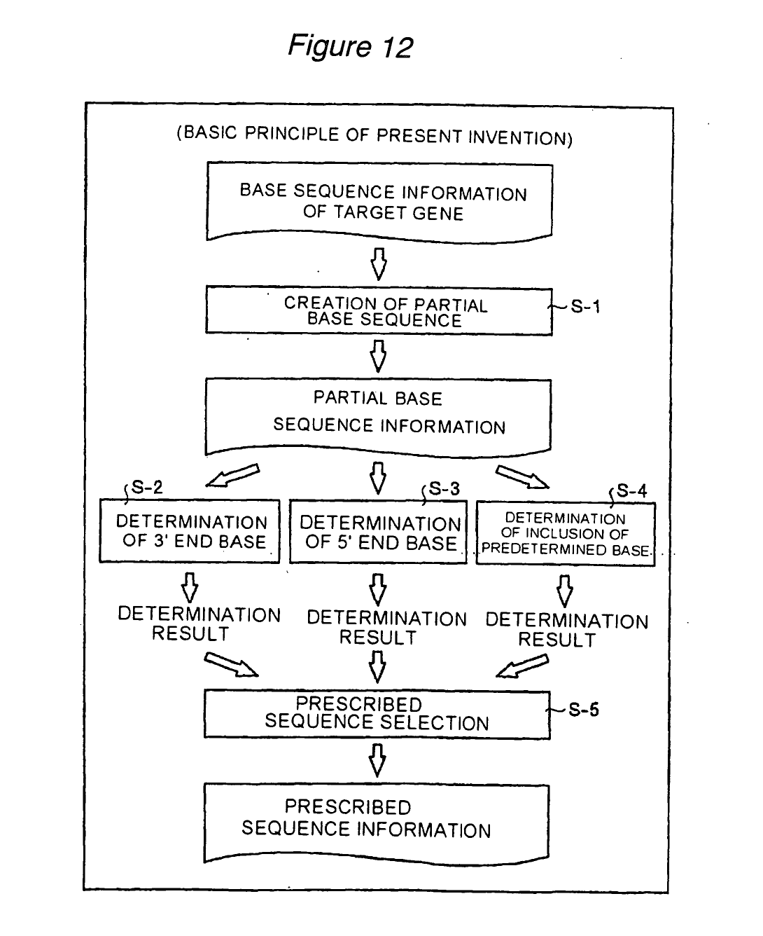

The summary of the present invention will be described below, and then the constitution, processing, etc., of the present invention will be described in detail. FIG. 12 is a principle diagram showing the basic principle of the present invention.

Overall, the present invention has the following basic features. That is, in the present invention, base sequence information of a target gene for RNA interference is obtained, and partial base sequence information corresponding to a sequence segment having a predetermined number of bases in the base sequence information is created (step S-1).

In step S-1, partial base sequence information having a predetermined number of bases may be created from a segment corresponding to a coding region or transcription region of the target gene in the base sequence information. Furthermore, partial base sequence information having a predetermined number of bases which is common in a plurality of base sequence information derived from different organisms (e.g., human base sequence information and mouse base sequence information) may be created. Furthermore, partial base sequence information having a predetermined number of bases which is common in a plurality of analogous base sequence information in the same species may be created. Furthermore, common partial base sequence information having a predetermined number of bases may be created from segments corresponding to coding regions or transcription regions of the target gene in a plurality of base sequence information derived from different species. Furthermore, common partial base sequence information having a predetermined number of bases may be created from segments corresponding to coding regions or transcription regions of the target gene in a plurality of analogous base sequence information in the same species. Consequently, a prescribed sequence which specifically causes RNA interference in the target gene can be efficiently selected, and calculation load can be reduced.

Furthermore, in step S-1, partial base sequence information including an overhanging portion may be created. Specifically, for example, partial base sequence information to which overhanging portion inclusion information, which shows that an overhanging portion is included, is added may be created. Namely, partial base sequence information and overhanging portion inclusion information may be correlated with each other. Thereby, it becomes possible to select the prescribed sequence with the overhanging portion being included from the start to perform designing.

The upper limit of the predetermined number of bases is, in the case of not including the overhanging portion, preferably 28 or less, more preferably 22 or less, and still more preferably 20 or less, and in the case of including the overhanging portion, preferably 32 or less, more preferably 26 or less, and still more preferably 24 or less. The lower limit of the predetermined number of bases is, in the case of not including the overhanging portion, preferably at least 13, more preferably at least 16, and still more preferably at least 18, and in the case of including the overhanging portion, preferably at least 17, more preferably at least 20, and still more preferably at least 22. Most preferably, the predetermined number of bases is, in the case of not including the overhanging portion, 19, and in the case of including the overhanging portion, 23. Thereby, it is possible to efficiently select the prescribed sequence which causes RNA interference without causing cytotoxicity even in mammals.

Subsequently, it is determined whether the 3′ end base in the partial base sequence information created in step S-1 is adenine, thymine, or uracil (step S-2). Specifically, for example, when the 3′ end base is adenine, thymine, or uracil, “1” may be outputted as the determination result, and when it is not, “0” may be outputted.

Subsequently, it is determined whether the 5′ end base in the partial base sequence information created in step S-1 is guanine or cytosine (step S-3). Specifically, for example, when the 5′ end base is guanine or cytosine, “1” may be outputted as the determination result, and when it is not, “0” may be outputted.

Subsequently, it is determined whether base sequence information comprising 7 bases at the 3′ end in the partial base sequence information created in step S-1 is rich in one or more types of bases selected from the group consisting of adenine, thymine, and uracil (step S-4). Specifically, for example, the number of bases of one or more types of bases selected from the group consisting of adenine, thymine, and uracil contained in the base sequence information comprising 7 bases at the 3′ end in the partial base sequence information may be outputted as the determination result. The rule of determination in step S-4 regulates that base sequence information in the vicinity of the 3′ end of the partial base sequence information created in step S-1 contains a rich amount of one or more types of bases selected from the group consisting of adenine, thymine, and uracil, and more specifically, as an index for search, regulates that the base sequence information in the range from the 3′ end base to the seventh base from the 3′ end is rich in one or more types of bases selected from the group consisting of adenine, thymine, and uracil.

In step S-4, the phrase “base sequence information rich in” corresponds to the phrase “sequence rich in” described in the column <1> Method for searching target base sequence for RNA interference. Specifically, for example, when the partial base sequence information created in step S-1 comprises about 19 bases, in the base sequence information comprising 7 bases in the partial base sequence information, preferably at least 3 bases, more preferably at least 4 bases, and particularly preferably at least 5 bases, are one or more types of bases selected from the group consisting of adenine, thymine, and uracil.

Furthermore, in steps S-2 to S-4, when partial base sequence information including the overhanging portion is determined, the sequence segment excluding the overhanging portion in the partial base sequence information is considered as the determination target.

Subsequently, based on the determination results in steps S-2, S-3, and S-4, prescribed sequence information which specifically causes RNA interference in the target gene is selected from the partial base sequence information created in step S-1 (Step S-5).

Specifically, for example, partial base sequence information in which the 3′ end base has been determined as adenine, thymine, or uracil in step S-2, the 5′ end base has been determined as guanine or cytosine in step S-3, and base sequence information comprising 7 bases at the 3′ end in the partial base sequence information has been determined as being rich in one or more types of bases selected from the group consisting of adenine, thymine, and uracil is selected as prescribed sequence information. Specifically, for example, a product of the values outputted in steps S-2, S-3, and S-4 may be calculated, and based on the product, prescribed sequence information may be selected from the partial base sequence information created in step S-1.

Consequently, it is possible to efficiently and easily produce a siRNA sequence which has an extremely high probability of causing RNA interference, i.e., which is effective for RNA interference, in mammals, etc.

Here, an overhanging portion may be added to at least one end of the prescribed sequence information selected in step S-5. Additionally, for example, when a target is searched, the overhanging portion may be added to both ends of the prescribed sequence information. Consequently, designing of a polynucleotide which causes RNA interference can be simplified.

Additionally, the number of bases in the overhanging portion corresponds to the number of bases described in the column <2> Method for designing base sequence of polynucleotide for causing RNA interference. Specifically, for example, 2 is particularly suitable as the number of bases.

Furthermore, base sequence information that is identical or similar to the prescribed sequence information selected in step S-5 may be searched from other base sequence information (e.g., base sequence information published in a public database, such as RefSeq (Reference Sequence project) of NCBI) using a known homology search method, such as BLAST, FASTA, or ssearch, and based on the searched identical or similar base sequence information, evaluation may be made whether the prescribed sequence information targets genes unrelated to the target gene.

Specifically, for example, base sequence information that is identical or similar to the prescribed sequence information selected in step S-5 is searched from other base sequence information (e.g., base sequence information published in a public database, such as RefSeq of NCBI) using a known homology search method, such as BLAST, FASTA, or ssearch. Based on the total amount of base sequence information on the genes unrelated to the target gene in the searched identical or similar base sequence information and the values showing the degree of identity or similarity (e.g., “E value” in BLAST, FASTA, or ssearch) attached to the base sequence information on the genes unrelated to the target gene, the total sum of the reciprocals of the values showing the degree of identity or similarity is calculated, and based on the calculated total sum (e.g., based on the size of the total sum calculated), evaluation may be made whether the prescribed sequence information targets genes unrelated to the target gene.

Consequently, it is possible to select a sequence which specifically causes RNA interference only to the target gene.

If RNA is synthesized based on the prescribed sequence information which is selected in accordance with the present invention and which does not cause RNA interference in genes unrelated to the target gene, it is possible to greatly reduce effort, time, and cost required compared with conventional techniques.

### [System Configuration]

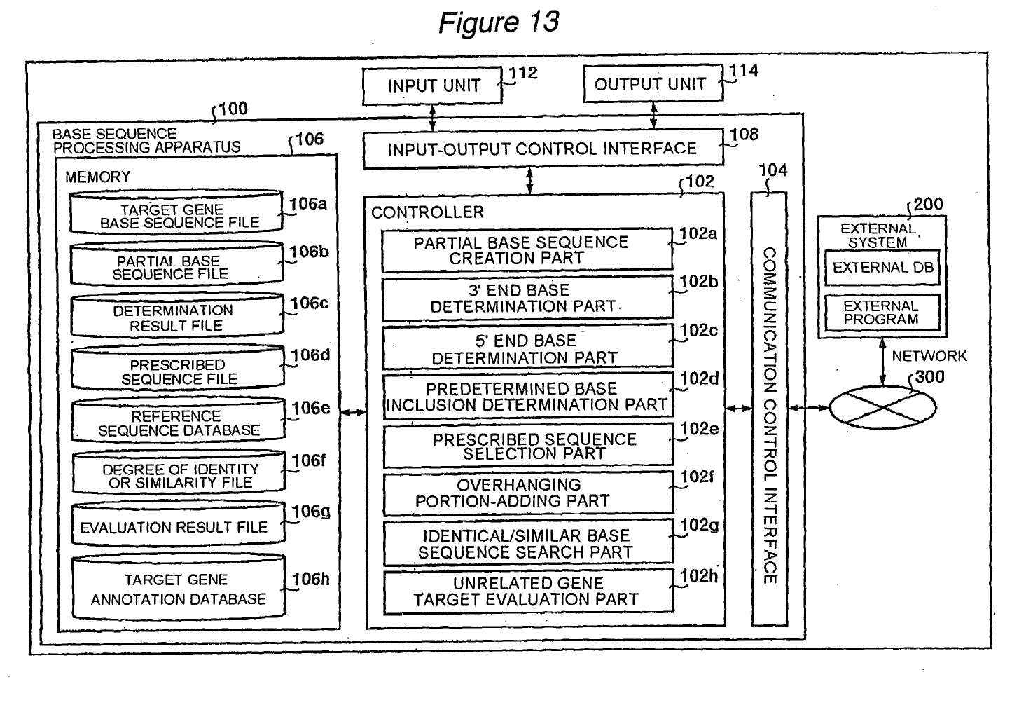

First, the configuration of this system will be described. FIG. 13 is a block diagram which shows an example of the system to which the present invention is applied and which conceptually shows only the parts related to the present invention.

Schematically, in this system, a base sequence processing apparatus 100 which processes base sequence information of a target gene for RNA interference and an external system 200 which provides external databases regarding sequence information, structural information, etc., and external programs, such as homology search, are connected to each other via a network 300 in a communicable manner.

In FIG. 13, the network 300 has a function of interconnecting between the base sequence processing apparatus 100 and the external system 200, and is, for example, the Internet.

In FIG. 13, the external system 200 is connected to the base sequence processing apparatus 100 via the network 300, and has a function of providing the user with the external databases regarding sequence information, structural information, etc., and Web sites which execute external programs, such as homology search and motif search.

The external system 200 may be constructed as a WEB server, ASP server, or the like, and the hardware structure thereof may include a commercially available information processing apparatus, such as a workstation or a personal computer, and its accessories. Individual functions of the external system 200 are implemented by a CPU, a disk drive, a memory unit, an input unit, an output unit, a communication control unit, etc., and programs for controlling them in the hardware structure of the external system 200.

In FIG. 13, the base sequence processing apparatus 100 schematically includes a controller 102, such as a CPU, which controls the base sequence processing apparatus 100 overall; a communication control interface 104 which is connected to a communication device (not shown in the drawing), such as a router, connected to a communication line or the like; an input-output control interface 108 connected to an input unit 112 and an output unit 114; and a memory 106 which stores various databases and tables. These parts are connected via given communication channels in a communicable manner. Furthermore, the base sequence processing apparatus 100 is connected to the network 300 in a communicable manner via a communication device, such as a router, and a wired or radio communication line.

Various databases and tables (a target gene base sequence file 106a˜a target gene annotation database 106h) which are stored in the memory 106 are storage means, such as fixed disk drives, for storing various programs used for various processes, tables, files, databases, files for web pages, etc.

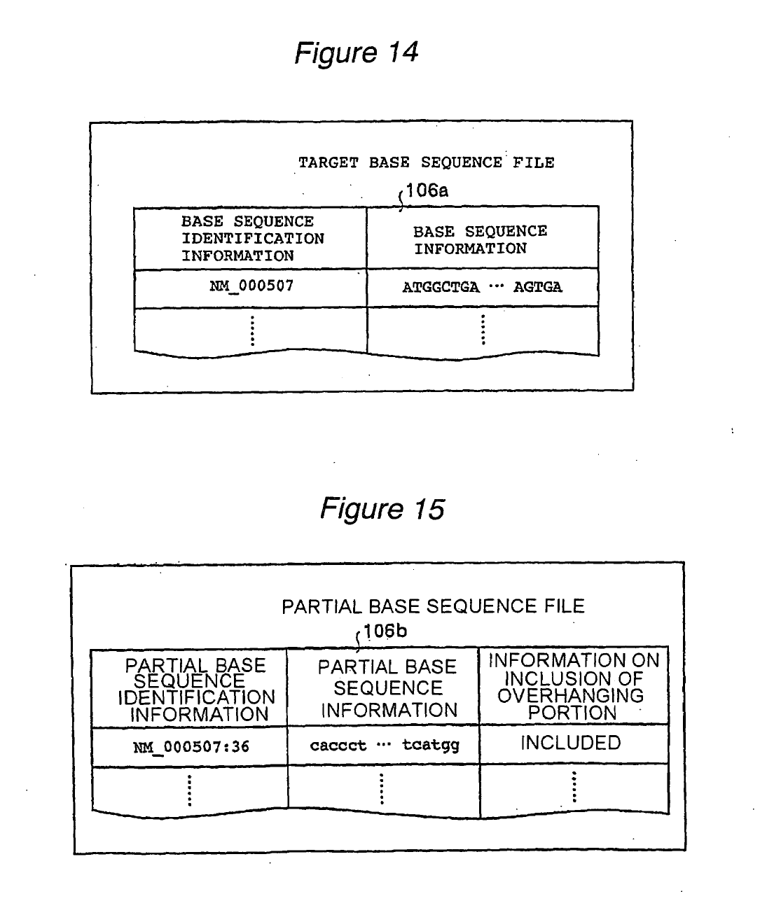

Among these components of the memory 106, the target gene base sequence file 106a is target gene base sequence storage means for storing base sequence information of the target gene for RNA interference. FIG. 14 is a diagram which shows an example of information stored in the target gene base sequence file 106a.

As shown in FIG. 14, the information stored in the target gene base sequence file 106a consists of base sequence identification information which uniquely identifies base sequence information of the target gene for RNA interference (e.g., “NM_{—}000507” in FIG. 14) and base sequence information (e.g., “ATGGCTGA . . . AGTGA” in FIG. 14), the base sequence identification information and the base sequence information being associated with each other.

Furthermore, a partial base sequence file 106b is partial base sequence storage means for storing partial base sequence information, i.e., a sequence segment having a predetermined number of bases in base sequence information of the target gene for RNA interference. FIG. 15 is a diagram which shows an example of information stored in the partial base sequence file 106b.

As shown in FIG. 15, the information stored in the partial base sequence file 106b consists of partial base sequence identification information which uniquely identifies partial base sequence information (e.g., “NM_{—}000507:36” in FIG. 15), partial base sequence information (e.g., “caccct . . . tcatgg” in FIG. 15), and information on inclusion of an overhanging portion which shows the inclusion of the overhanging portion (e.g., “included” in FIG. 15), the partial base sequence identification information, the partial base sequence information, and the information on inclusion of the overhanging portion being associated with each other.

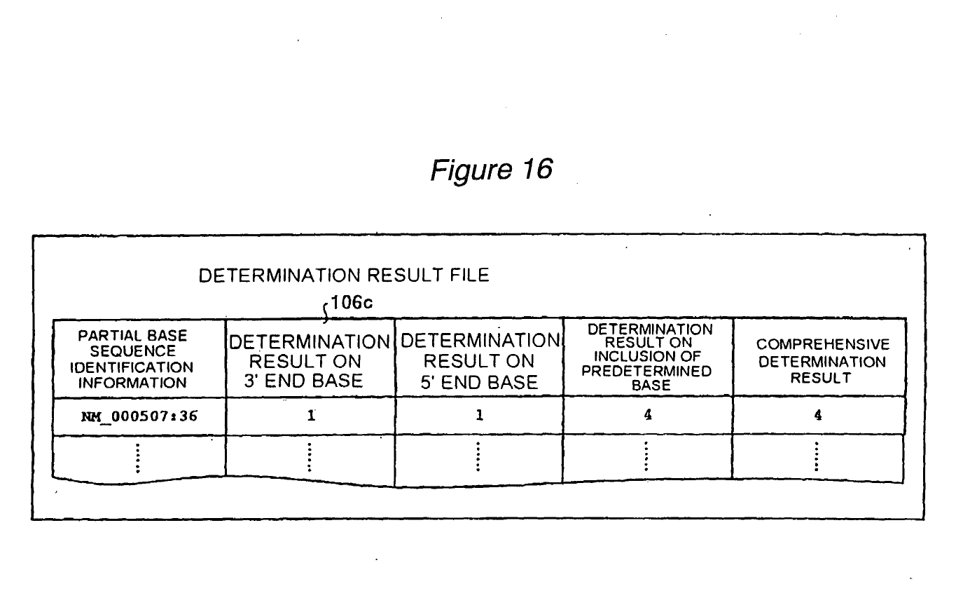

A determination result file 106c is determination result storage means for storing the results determined by a 3′ end base determination part 102b, a 5′ end base determination part 102c, and a predetermined base inclusion determination part 102d, which will be described below. FIG. 16 is a diagram which shows an example of information stored in the determination result file 106c.

As shown in FIG. 16, the information stored in the determination result file 106c consists of partial base sequence identification information (e.g., “NM_{—}000507:36” in FIG. 16), determination result on 3′ end base corresponding to a result determined by the 3′ end base determination part 102b (e.g., “1” in FIG. 16), determination result on 5′ end base corresponding to a result determined by the 5′ end base determination part 102c (e.g., “1” in FIG. 16), determination result on inclusion of predetermined base corresponding to a result determined by the predetermined base inclusion determination part 102d (e.g., “4” in FIG. 16), and comprehensive determination result corresponding to a result obtained by putting together the results determined by the 3′ end base determination part 102b, the 5′ end base determination part 102c, and the predetermined base inclusion determination part 102d (e.g., “4” in FIG. 16), the partial base sequence identification information, the determination result on 3′ end base, the determination result on 5′ end base, the determination result on inclusion of predetermined base, and the comprehensive determination result being associated with each other.

Additionally, FIG. 16 shows an example of the case in which, with respect to the determination result on 3′ end base and the determination result on 5′ end base, “1” is set when determined as being “included” by each of the 3′ end base determination part 102b and the 5′ end base determination part 102c and “0” is set when determined as being “not included”. Furthermore, FIG. 16 shows an example of the case in which the determination result on inclusion of predetermined base is set as the number of bases corresponding to one or more types of bases selected from the group consisting of adenine, thymine, and uracil contained in the base sequence information comprising 7 bases at the 3′ end in the partial base sequence information. Furthermore, FIG. 16 shows an example of the case in which the comprehensive determination result is set as the product of the determination result on 3′ end base, the determination result on 5′ end base, and the determination result on inclusion of predetermined base. Specifically, for example, when the product is 3 or less, “0” may be set.

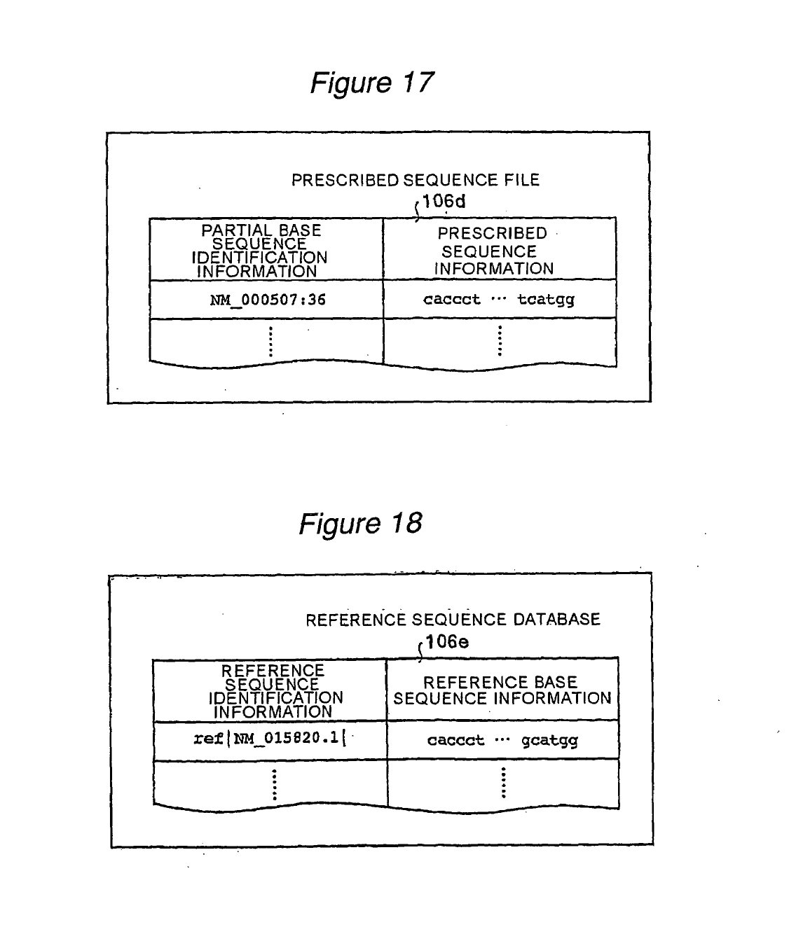

Furthermore, a prescribed sequence file 106d is prescribed sequence storage means for storing prescribed sequence information corresponding to partial base sequence information which specifically causes RNA interference in the target gene. FIG. 17 is a diagram which shows an example of information stored in the prescribed sequence file 106d.

As shown in FIG. 17, the information stored in the prescribed sequence file 106d consists of partial base sequence identification information (e.g., “NM_{—}000507:36” in FIG. 17) and prescribed sequence information corresponding to partial base sequence information which specifically causes RNA interference in the target gene (e.g., caccct . . . tcatgg” in FIG. 17), the partial base sequence identification information and the prescribed sequence information being associated with each other.

Furthermore, a reference sequence database 106e is a database which stores reference base sequence information corresponding to base sequence information to which reference is made to search base sequence information identical or similar to the prescribed sequence information by an identical/similar base sequence search part 102g, which will be described below. The reference sequence database 106e may be an external base sequence information database accessed via the Internet or may be an in-house database created by copying such a database, storing the original sequence information, or further adding unique annotation information to such a database. FIG. 18 is a diagram which shows an example of information stored in the reference sequence database 106e.

As shown in FIG. 18, the information stored in the reference sequence database 106e consists of reference sequence identification information (e.g., “ref|NM_{—}015820.1|” in FIG. 18) and reference base sequence information (e.g., “caccct . . . gcatgg” in FIG. 18), the reference sequence identification information and the reference base sequence information being associated with each other.

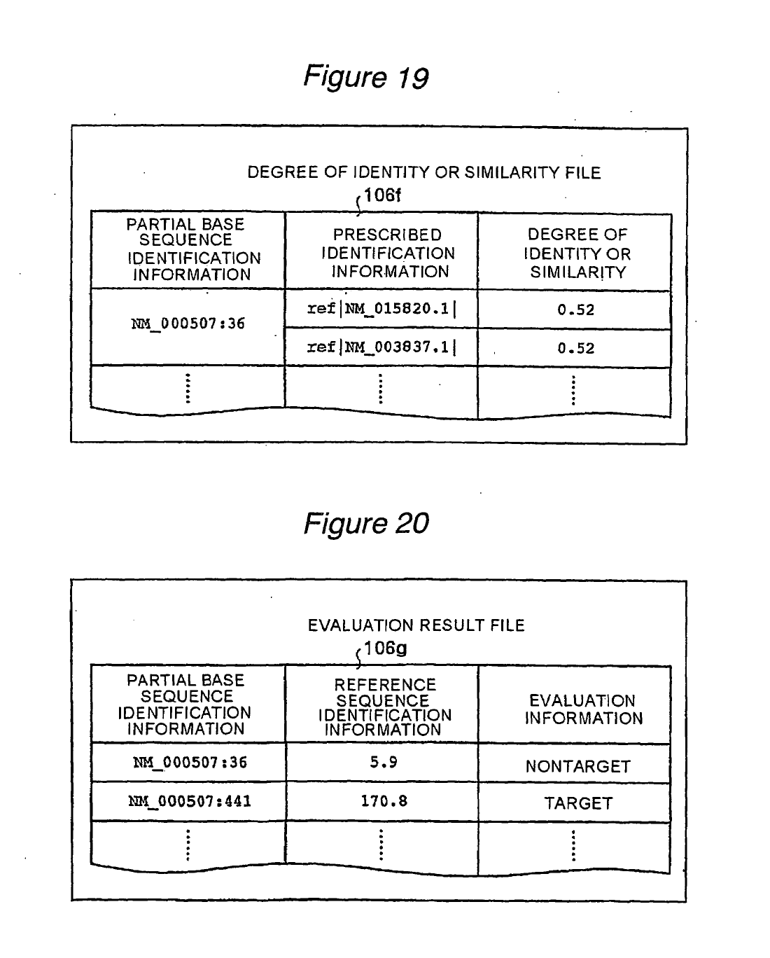

Furthermore, a degree of identity or similarity file 106f is degree of identity or similarity storage means for storing the degree of identity or similarity corresponding to a degree of identity or similarity of identical or similar base sequence information searched by an identical/similar base sequence search part 102g, which will be described below. FIG. 19 is a diagram which shows an example of information stored in the degree of identity or similarity file 106f.

As shown in FIG. 19, the information stored in the degree of identity or similarity file 106f consists of partial base sequence identification information (e.g., “NM_{—}000507:36” in FIG. 19), reference sequence identification information (e.g., “ref|NM_{—}015820.1|” and “ref|NM_{—}003837.1|” in FIG. 19), and degree of identity or similarity (e.g., “0.52” in FIG. 19), the partial base sequence identification information, the reference sequence identification information, and the degree of identity or similarity being associated with each other.

Furthermore, an evaluation result file 106g is evaluation result storage means for storing the result of evaluation on whether genes unrelated to the target gene are targeted by an unrelated gene target evaluation part 102h, which will be described below. FIG. 20 is a diagram which shows an example of information stored in the evaluation result file 106g.

As shown in FIG. 20, the information stored in the evaluation result file 106g consists of partial base sequence identification information (e.g., “NM_{—}000507:36” and “NM_{—}000507:441” in FIG. 20), total sum calculated by a total sum calculation part 102m, which will be described below, (e.g., “5.9” and “170.8” in FIG. 20), and evaluation result (e.g., “nontarget” and “target” in FIG. 20), the partial base sequence identification information, the total sum, and the evaluation result being associated with each other. Additionally, in FIG. 20, “nontarget” means that the prescribed sequence information does not target genes unrelated to the target gene, and “target” means that the prescribed sequence information targets genes unrelated to the target gene.

A target gene annotation database 106h is target gene annotation storage means for storing annotation information regarding the target gene. The target gene annotation database 106h may be an external annotation database which stores annotation information regarding genes and which is accessed via the Internet or may be an in-house database created by copying such a database, storing the original sequence information, or further adding unique annotation information to such a database.

The information stored in the target gene annotation database 106h consists of target gene identification information which identifies the target gene (e.g., the name of a gene to be targeted, and Accession number (e.g., “NM_{—}000507” and “FBP1” described on the top in FIG. 3)) and simplified information on the target gene (e.g., “Homo sapiens fructose-1,6-bisphosphatase 1” describe on the top in FIG. 3), the target gene identification information and the simplified information being associated with each other.

In FIG. 13, the communication control interface 104 controls communication between the base sequence processing apparatus 100 and the network 300 (or a communication device, such as a router). Namely, the communication control interface 104 performs data communication with other terminals via communication lines.

In FIG. 13, the input-output control interface 108 controls the input unit 112 and the output unit 114. Here, as the output unit 114, in addition to a monitor (including a home television), a speaker may be used (hereinafter, the output unit 114 may also be described as a monitor). As the input unit 112, a keyboard, a mouse, a microphone, or the like may be used. The monitor cooperates with a mouse to implement a pointing device function.

In FIG. 13, the controller 102 includes control programs, such as OS (Operating System), programs regulating various processing procedures, etc., and internal memories for storing required data, and performs information processing for implementing various processes using the programs, etc. The controller 102 functionally includes a partial base sequence creation part 102a, a 3′ end base determination part 102b, a 5′ end base determination part 102c, a predetermined base inclusion determination part 102d, a prescribed sequence selection part 102e, an overhanging portion-adding part 102f, an identical/similar base sequence search part 102g, and an unrelated gene target evaluation part 102h.

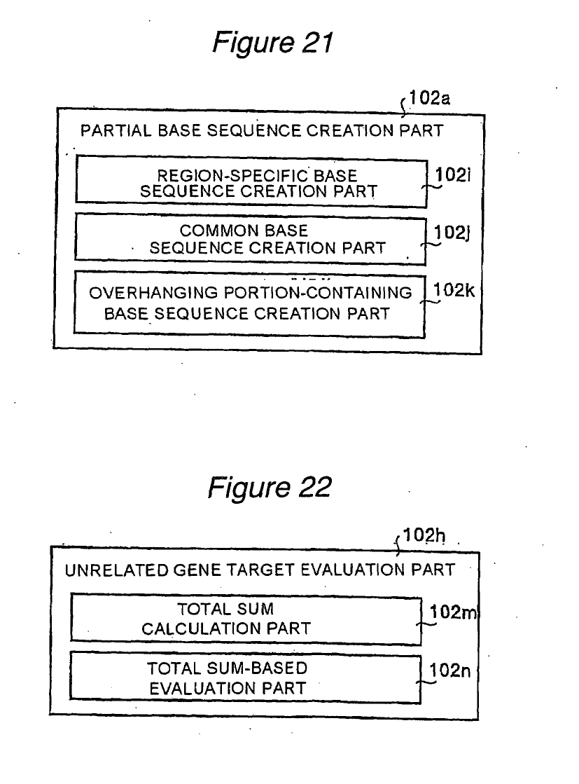

Among them, the partial base sequence creation part 102a is partial base sequence creation means for acquiring base sequence information of a target gene for RNA interference and creating partial base sequence information corresponding to a sequence segment having a predetermined number of bases in the base sequence information. As shown in FIG. 21, the partial base sequence creation part 102a includes a region-specific base sequence creation part 102i, a common base sequence creation part 102j, and an overhanging portion-containing base sequence creation part 102k.

FIG. 21 is a block diagram which shows an example of the structure of the partial base sequence creation part 102a of the system to which the present invention is applied and which shows only the parts related to the present invention.

In FIG. 21, the region-specific base sequence creation part 102i is region-specific base sequence creation means for creating partial base sequence information having a predetermined number of bases from a segment corresponding to a coding region or transcription region of the target gene in the base sequence information.

The common base sequence creation part 102j is common base sequence creation means for creating partial base sequence information having a predetermined number of bases which is common in a plurality of base sequence information derived from different organisms.

The overhanging portion-containing base sequence creation part 102k is overhanging portion-containing base sequence creation means for creating partial base sequence information containing an overhanging portion.

Referring back to FIG. 13, the 3′ end base determination part 102b is 3′ end base determination means for determining whether the 3′ end base in the partial base sequence information is adenine, thymine, or uracil.

Furthermore, the 5′ end base determination part 102c is 5′ end base determination means for determining whether the 5′ end base in the partial base sequence information is guanine or cytosine.

Furthermore, the predetermined base inclusion determination part 102d is predetermined base inclusion determination means for determining whether the base sequence information comprising 7 bases at the 3′ end in the partial base sequence information is rich in one or more types of bases selected from the group consisting of adenine, thymine, and uracil.

Furthermore, the prescribed sequence selection part 102e is prescribed sequence selection means for selecting prescribed sequence information, which specifically causes RNA interference in the target gene, from the partial base sequence information based on the results determined by the 3′ end base determination part 102b, the 5′ end base determination part 102c, and the predetermined base inclusion determination part 102c.

Furthermore, the overhanging portion-adding part 102f is overhanging portion addition means for adding an overhanging portion to at least one end of the prescribed sequence information.

Furthermore, the identical/similar base sequence search part 102g is identical/similar base sequence search means for searching base sequence information, identical or similar to the prescribed sequence information, from other base sequence information.

Furthermore, the unrelated gene target evaluation part 102h is unrelated gene target evaluation means for evaluating whether the prescribed sequence information targets genes unrelated to the target gene based on the identical or similar base sequence information. As shown in FIG. 22, the unrelated gene target evaluation part 102h further includes a total sum calculation part 102m and a total sum-based evaluation part 102n.

FIG. 22 is a block diagram which shows an example of the structure of the unrelated gene target evaluation part 102h of the system to which the present invention is applied and which schematically shows only the parts related to the present invention.

In FIG. 22, the total sum calculation part 102m is total sum calculation means for calculating the total sum of reciprocals of the values showing the degree of identity or similarity based on the total amount of base sequence information on the genes unrelated to the target gene in identical or similar base sequence information and the values showing the degree of identity or similarity attached to the base sequence information on the genes unrelated to the target gene (identity or similarity).

Furthermore, the total sum-based evaluation part 102n is total sum-based target evaluation means for evaluating whether the prescribed sequence information targets genes unrelated to the target gene based on the total sum calculated by the total sum calculation part 102m.

The details of processing of each part will be described later.

### [Processing of the System]

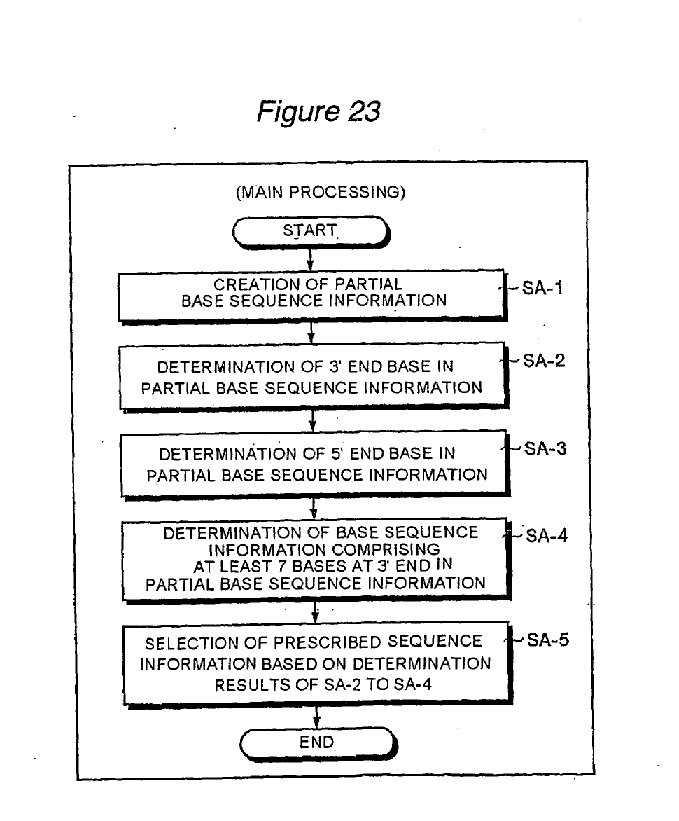

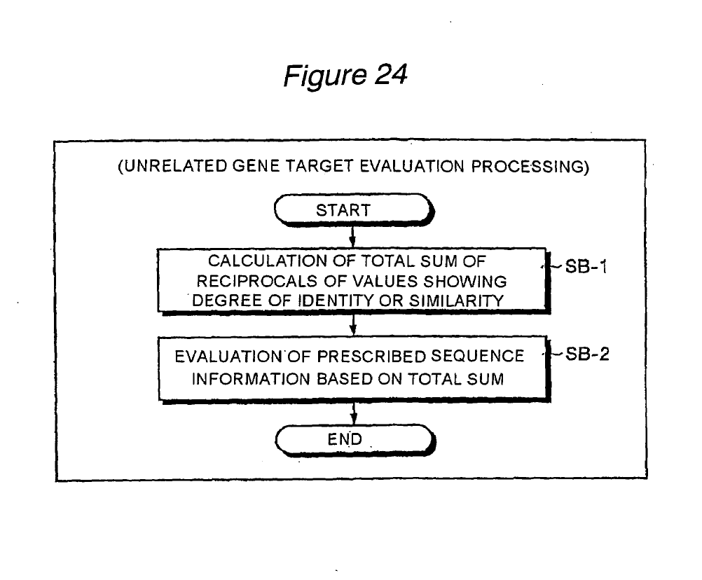

An example of processing of the system having the configuration described above in this embodiment will be described in detail with reference to FIGS. 23 and 24.

### [Main Processing]

First, the details of the main processing will be described with reference to FIG. 23, etc. FIG. 23 is a flowchart which shows an example of the main processing of the system in this embodiment.

The base sequence processing apparatus 100 acquires base sequence information of a target gene for RNA interference by the partial base sequence creation process performed by the partial base sequence creation part 102a, stores it in a predetermined memory region of the target gene base sequence file 106a, creates partial base sequence information corresponding to a sequence segment having a predetermined number of bases in the base sequence information, and stores the created partial base sequence information in a predetermined memory region of the partial base sequence file 106b (step SA-1).

In step SA-1, the partial base sequence creation part 102a may create partial base sequence information having a predetermined number of bases from a segment corresponding to a coding region or transcription region of the target gene in the base sequence information by the processing of the region-specific base sequence creation part 102i and may store the created partial base sequence information in a predetermined memory region of the partial base sequence file 106b.

In step SA-1, the partial base sequence creation part 102a may create partial base sequence information having a predetermined number of bases which is common in a plurality of base sequence information derived from different organisms (e.g., human base sequence information and mouse base sequence information) by the processing of the common base sequence creation part 102j and may store the created partial base sequence information in a predetermined memory region of the partial base sequence file 106b. Furthermore, common partial base sequence information having a predetermined number of bases which is common in a plurality of analogous base sequence information in the same species may be created.

In step SA-1, the partial base sequence creation part 102a may create partial base sequence information having a predetermined number of bases from segments corresponding to coding regions or transcription regions of the target gene in a plurality of base sequence information derived from different species by the processing of the region-specific base sequence creation part 102i and the common base sequence creation part 102j and may store the created partial base sequence information in a predetermined memory region of the partial base sequence file 106b. Furthermore, common partial base sequence information having a predetermined number of bases may be created from segments corresponding to coding regions or transcription regions of the target gene in a plurality of analogous base sequence information in the same species.

Furthermore, in step SA-1, the partial base sequence creation part 102a may create partial base sequence information containing an overhanging portion by the processing of the overhanging portion-containing base sequence creation part 102k. Specifically, for example, the partial base sequence creation part 102a may create partial base sequence information to which the overhanging portion inclusion information which shows the inclusion of the overhanging portion by the processing of the overhanging portion-containing base sequence creation part 102k and may store the created partial base sequence information and the overhanging portion inclusion information so as to be associated with each other in a predetermined memory region of the partial base sequence file 106b.

The upper limit of the predetermined number of bases is, in the case of not including the overhanging portion, preferably 28 or less, more preferably 22 or less, and still more preferably 20 or less, and in the case of including the overhanging portion, preferably 32 or less, more preferably 26 or less, and still more preferably 24 or less. The lower limit of the predetermined number of bases is, in the case of not including the overhanging portion, preferably at least 13, more preferably at least 16, and still more preferably at least 18, and in the case of including the overhanging portion, preferably at least 17, more preferably at least 20, and still more preferably at least 22. Most preferably, the predetermined number of bases is, in the case of not including the overhanging portion, 19, and in the case of including the overhanging portion, 23.

Subsequently, the base sequence processing apparatus 100 determines whether the 3′ end base in the partial base sequence information created in step SA-1 is adenine, thymine, or uracil by the processing of the 3′ end base determination part 102b and stores the determination result in a predetermined memory region of the determination result file 106c (step SA-2). Specifically, for example, the base sequence processing apparatus 100 may store “1” when the 3′ end base in the partial base sequence information created in step SA-1 is adenine, thymine, or uracil, by the processing of the 3′ end base determination part 102b, and “0” when it is not, in a predetermined memory region of the determination result file 106c.

Subsequently, the base sequence processing apparatus 100 determines whether the 5′ end base in the partial base sequence information created in step SA-1 is guanine or cytosine by the processing of the 5′ end base determination part 102c and stores the determination result in a predetermined memory region of the determination result file 106c (step SA-3). Specifically, for example, the base sequence processing apparatus 100 may store “1” when the 5′ end base in the partial base sequence information created in step SA-1 is guanine or cytosine, by the processing of the 5′ end base determination part 102c, and “0” when it is not, in a predetermined memory region of the determination result file 106c.

Subsequently, the base sequence processing apparatus 100 determines whether the base sequence information comprising 7 bases at the 3′ end in the partial base sequence information created in step SA-1 is rich in one or more types of bases selected from the group consisting of adenine, thymine, and uracil by the processing of the predetermined base inclusion determination part 102d and stores the determination result in a predetermined memory region of the determination result file 106c (step SA-4). Specifically, for example, the base sequence processing apparatus 100, by the processing of the predetermined base inclusion determination part 102d, may store the number of bases corresponding to one or more types of bases selected from the group consisting of adenine, thymine, and uracil contained in the base sequence information comprising 7 bases at the 3′ end in the partial base sequence information created in step SA-1 in a predetermined memory region of the determination result file 106c. The rule of determination in step SA-4 regulates that base sequence information in the vicinity of the 3′ end of the partial base sequence information created in step SA-1 contains a rich amount of one or more types of bases selected from the group consisting of adenine, thymine, and uracil, and more specifically, as an index for search, regulates that the base sequence information in the range from the 3′ end base to the seventh base from the 3′ end is rich in one or more types of bases selected from the group consisting of adenine, thymine, and uracil.

In step SA-4, the phrase “base sequence information rich in” corresponds to the phrase “sequence rich in” described in the column <1> Method for searching target base sequence for RNA interference. Specifically, for example, when the partial base sequence information created in step SA-1 comprises about 19 bases, in the base sequence information comprising 7 bases at the 3′ end in the partial base sequence information, preferably at least 3 bases, more preferably at least 4 bases, and particularly preferably at least 5 bases, are one or more types of bases selected from the group consisting of adenine, thymine, and uracil.

Furthermore, in steps SA-2 to SA-4, when partial base sequence information including the overhanging portion is determined, the sequence segment excluding the overhanging portion in the partial base sequence information is considered as the determination target.

Subsequently, based on the determination results in steps SA-2, SA-3, and SA-4, the base sequence processing apparatus 100, by the processing of the prescribed sequence selection part 102e, selects prescribed sequence information which specifically causes RNA interference in the target gene from the partial base sequence information created in step SA-1 and stores it in a predetermined memory region of the prescribed sequence file 106d (Step SA-5).

Specifically, for example, the base sequence processing apparatus 100, by the processing of the prescribed sequence selection part 102e, selects partial base sequence information, in which the 3′ end base has been determined as adenine, thymine, or uracil in step SA-2, the 5′ end base has been determined as guanine or cytosine in step SA-3, and base sequence information comprising 7 bases at the 3′ end in the partial base sequence information has been determined as being rich in one or more types of bases selected from the group consisting of adenine, thymine, and uracil, as prescribed sequence information, and stores it in a predetermined memory region of the prescribed sequence file 106d. Specifically, for example, the base sequence processing apparatus 100, by the processing of the prescribed sequence selection part 102e, may calculate a product of the values outputted in steps SA-2, SA-3, and SA-4 and, based on the product, select prescribed sequence information from the partial base sequence information created in step SA-1.

Here, the base sequence processing apparatus 100 may add an overhanging portion to at least one end of the prescribed sequence information selected in step SA-5 by the processing of the overhanging portion-adding part 102f, and may store it in a predetermined memory region of the prescribed sequence file 106d. Specifically, for example, by the processing of the overhanging portion-adding part 102f, the base sequence processing apparatus 100 may change the prescribed sequence information stored in the prescribed sequence information section in the prescribed sequence file 106d to prescribed sequence information in which an overhanging portion is added to at least one end. Additionally, for example, when a target is searched, the overhanging portion may be added to both ends of the prescribed sequence information.

Additionally, the number of bases in the overhanging portion corresponds to the number of bases described in the column <2> Method for designing base sequence of polynucleotide for causing RNA interference. Specifically, for example, 2 is particularly suitable as the number of bases.

Furthermore, the base sequence processing apparatus 100, by the processing of the identical/similar base sequence search part 102g, may search base sequence information that is identical or similar to the prescribed sequence information selected in step SA-5 from other base sequence information (e.g., base sequence information published in a public database, such as RefSeq of NCBI) using a known homology search method, such as BLAST, FASTA, or ssearch, and based on the searched identical or similar base sequence information, by the unrelated gene target evaluation process performed by the unrelated gene target evaluation part 102h, may evaluate whether the prescribed sequence information targets genes unrelated to the target gene.

Specifically, for example, the base sequence processing apparatus 100, by the processing of the identical/similar base sequence search part 102g, may search base sequence information that is identical or similar to the prescribed sequence information selected in step SA-5 from other base sequence information (e.g., base sequence information published in a public database, such as RefSeq of NCBI) using a known homology search method, such as BLAST, FASTA, or ssearch. The unrelated gene target evaluation part 102h, by the processing of the total sum calculation part 102m, may calculate the total sum of the reciprocals of the values showing the degree of identity or similarity based on the total amount of base sequence information on the genes unrelated to the target gene in the searched identical or similar base sequence information and the values showing the degree of identity or similarity (e.g., “E value” in BLAST, FASTA, or ssearch) attached to the base sequence information on the genes, unrelated to the target gene. The unrelated gene target evaluation part 102h, by the processing of the total sum-based evaluation part 102n, may evaluate whether the prescribed sequence information targets genes unrelated to the target gene based on the calculated total sum.

Here, the details of the unrelated gene target evaluation process performed by the unrelated gene target evaluation part 102h will be described with reference to FIG. 24.

FIG. 24 is a flowchart which shows an example of the unrelated gene evaluation process of the system in this embodiment.

First, the base sequence processing apparatus 100, by the processing of the identical/similar base sequence search part 102g, searches base sequence information that is identical or similar to the prescribed sequence information selected in step SA-5 from other base sequence information (e.g., base sequence information published in a public database, such as RefSeq of NCBI) using a known homology search method, such as BLAST, FASTA, or ssearch, and stores identification information of the prescribed sequence information (“partial base sequence identification information” in FIG. 19), identification information of the searched identical or similar base sequence information (“reference sequence identification information” in FIG. 19), and the value showing the degree of identity or similarity (e.g., “E value” in BLAST, FASTA, or ssearch) (“degree of identity or similarity” in FIG. 19) attached to the searched identical or similar base sequence information so as to be associated with each other in a predetermined memory region of the degree of identity or similarity file 106f.

Subsequently, the unrelated gene target evaluation part 102h, by the processing of the total sum calculation part 102m, calculates the total sum of reciprocals of the values showing the degree of identity or similarity based on the total amount of base sequence information on the genes unrelated to the target gene in the searched identical or similar base sequence information and the values showing the degree of identity or similarity (e.g., “E value” in BLAST, FASTA, or ssearch) attached to the base sequence information on the genes unrelated to the target gene, and stores identification information of the prescribed sequence information (“partial base sequence identification information” in FIG. 20) and the calculated total sum (“total sum” in FIG. 20) so as to be associated with each other in a predetermined memory region of the evaluation result file 106g (step SB-1).

Subsequently, the unrelated gene target evaluation part 102h, by the processing of the total sum-based evaluation part 102n, evaluates whether the prescribed sequence information targets genes unrelated to the target gene based on the total sum calculated in step SB-1 (e.g., based on the size of the total sum calculated in step SB-1), and stores the evaluation results (“nontarget” and “target” in FIG. 20) in a predetermined memory region of the evaluation result file 106g (Step SB-2).

The main process is thereby completed.

### <8> Pharmaceutical Composition

The present invention also provides a pharmaceutical composition comprising a pharmaceutically effective amount of the polynucleotide of the present invention. The use of the pharmaceutical composition of the present invention is not particularly limited. Since the pharmaceutical composition inhibits, through RNAi, the expression of a gene containing a target sequence of each polynucleotide, which is an active ingredient, it is useful in preventing and/or treating diseases in which such genes are involved.

The sequence to be targeted by the polynucleotide contained in the pharmaceutical composition of the present invention is a sequence selected as a prescribed sequence conforming to the above rules (a) to (f). Preferably, such a sequence may be any of SEQ ID NOs: 47 to 817081. In particular, if the target sequence is a sequence highly specific to the target gene, the polynucleotide selectively produces an inhibitory effect only on the expression of the target gene containing the target sequence, but not on the other genes (i.e., the polynucleotide has less off-target effect), thus reducing influences of side effects, etc. It is therefore more preferred that the target sequence of the polynucleotide has high specificity to the target gene. Among the selected sequences (e.g., SEQ ID NOs: 47 to 817081), a sequence whose off-target effect can be further reduced is preferred as a prescribed sequence conforming to the above rules (a) to (f). As a preferred prescribed sequence of the target gene, it is possible to select a sequence which contains mismatches of at least 3 bases against the base sequences of other genes and for which there is only a minimum number of other genes having a base sequence containing mismatches of at least 3 bases. The requirement “there is only a minimum number of other genes” means that “other genes having a base sequence containing mismatches of at least 3 bases” (i.e., similar genes) are as few in number as possible; for example, there are preferably 10 or less genes, more preferably 6 or less genes, still more preferably only one gene, or most preferably no gene.

For example, the 53998 sequences shown in FIG. 46 are obtained among SEQ ID NOs: 47 to 817081 by selecting sequences which contain mismatches of 3 bases against the base sequences of other genes (i.e., prescribed sequences of 19 bases in which 16 bases other than these 3 mismatched bases are the same as those of other genes) and for which there is only a minimum number of other genes having a base sequence containing mismatches of 3 bases. Thus, the target sequence is particularly preferably any of these sequences. With respect to these sequences, most of their relationships have been identified, such as genes containing these target sequences, disease names related to these genes, biological function categories according to GO_ID of these genes in Gene Ontology, and biological functions reported in documents. These relationships are shown in FIG. 46. The polynucleotide of the present invention inhibits the expression of a gene containing a target sequence through RNAi, and hence allows treatment and/or prevention of diseases related to the gene and control of its biological functions. Once a target sequence of the polynucleotide has been identified on the basis of the disclosures of the present specification, drawings and so on, those skilled in the art will readily understand diseases and/or biological functions on which the polynucleotide produces an effect.

Thus, the pharmaceutical composition of the present invention is preferably useful in treating and/or preventing the diseases listed in the column “Related Disease” of FIG. 46 or diseases associated with the gene-related biological functions listed in the columns “Biological Function Category” and/or “Reported Biological Function” of FIG. 46.

The pharmaceutical composition of the present invention is more preferably useful in treating and/or preventing a disease in which a gene belonging to any of the following 1) to 9) is involved:

1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or

### 9) G protein-coupled receptor or a G protein-coupled receptor-related gene.

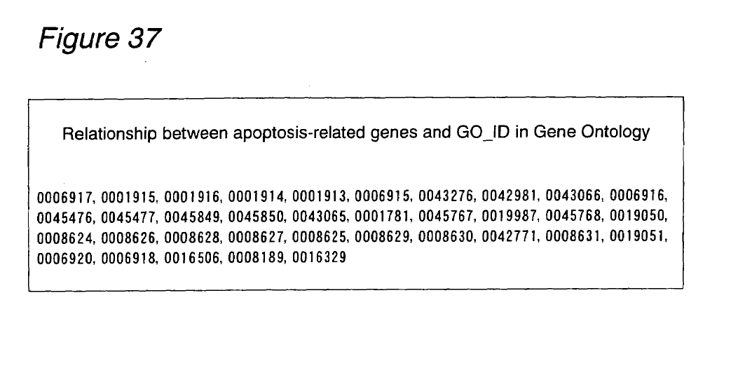

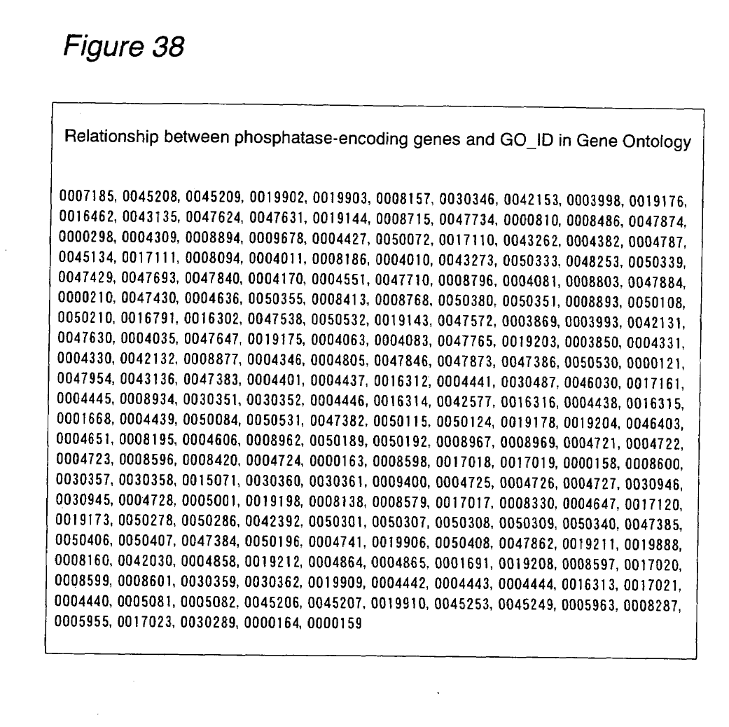

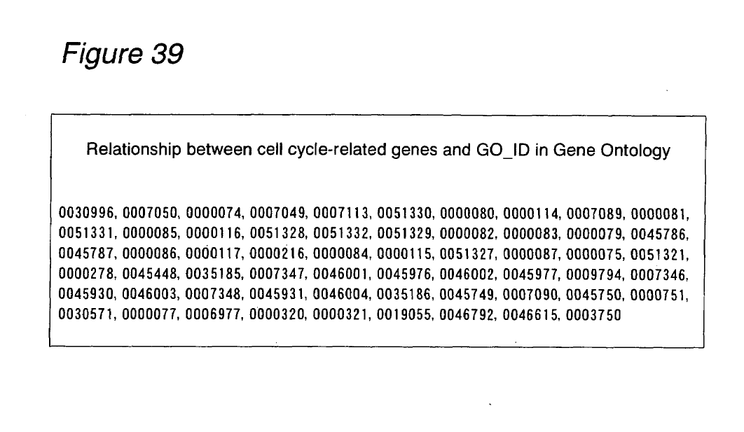

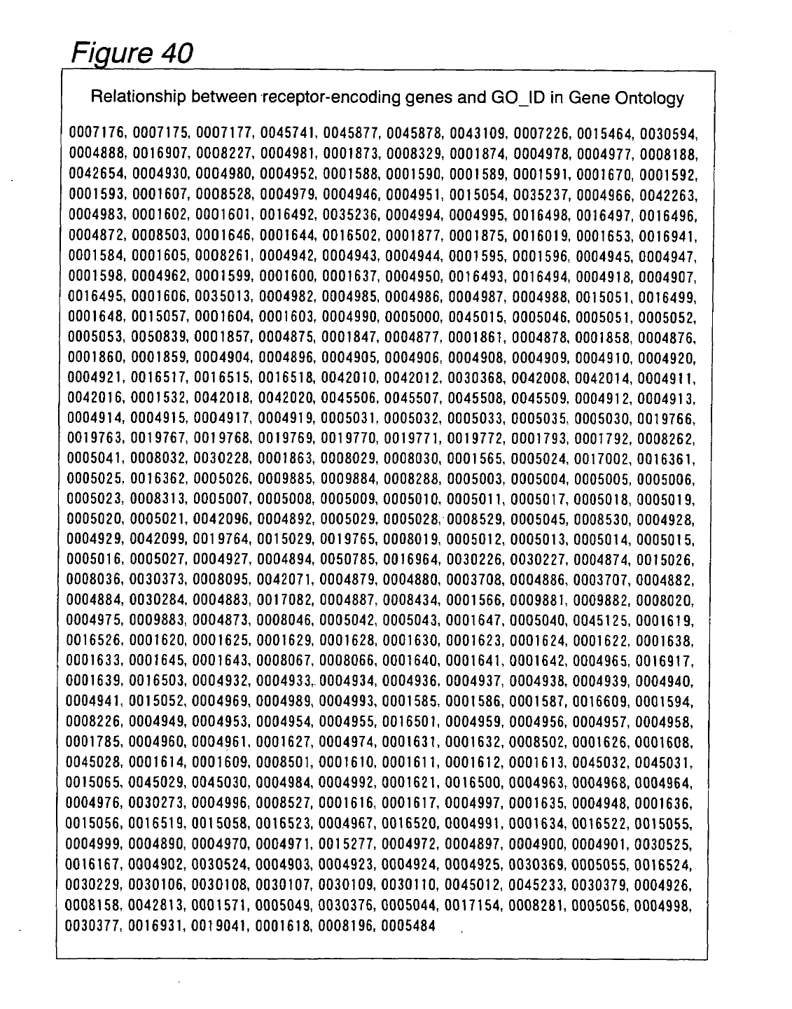

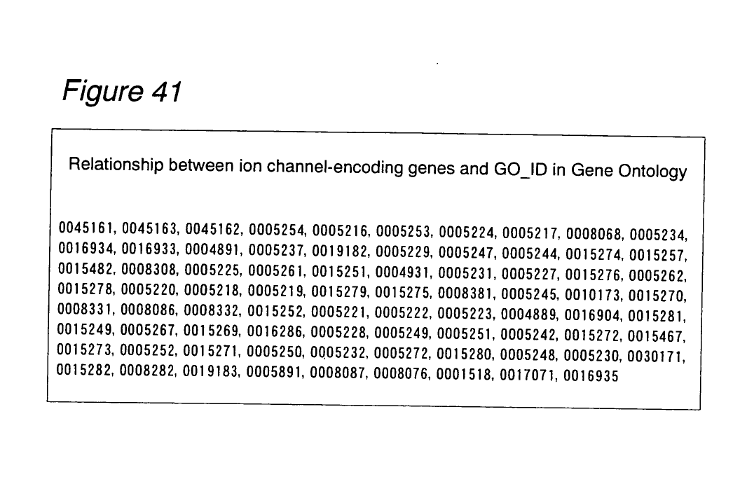

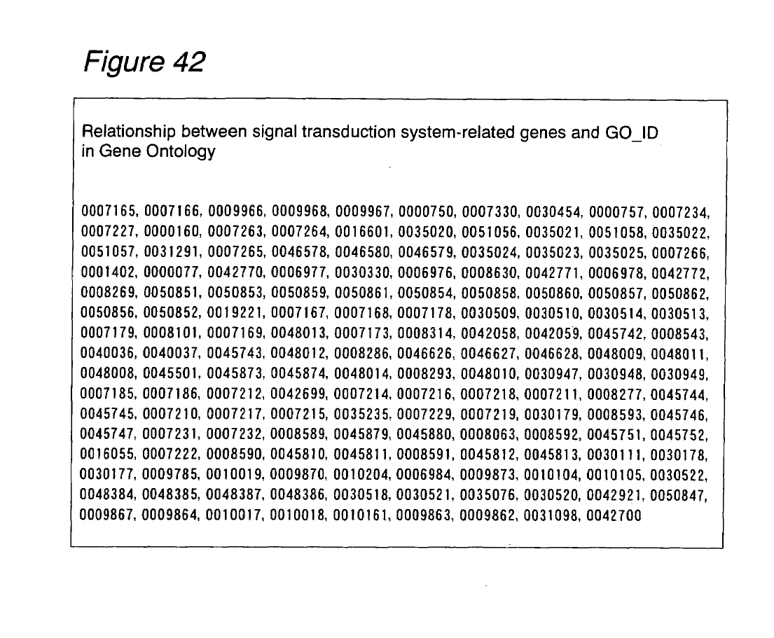

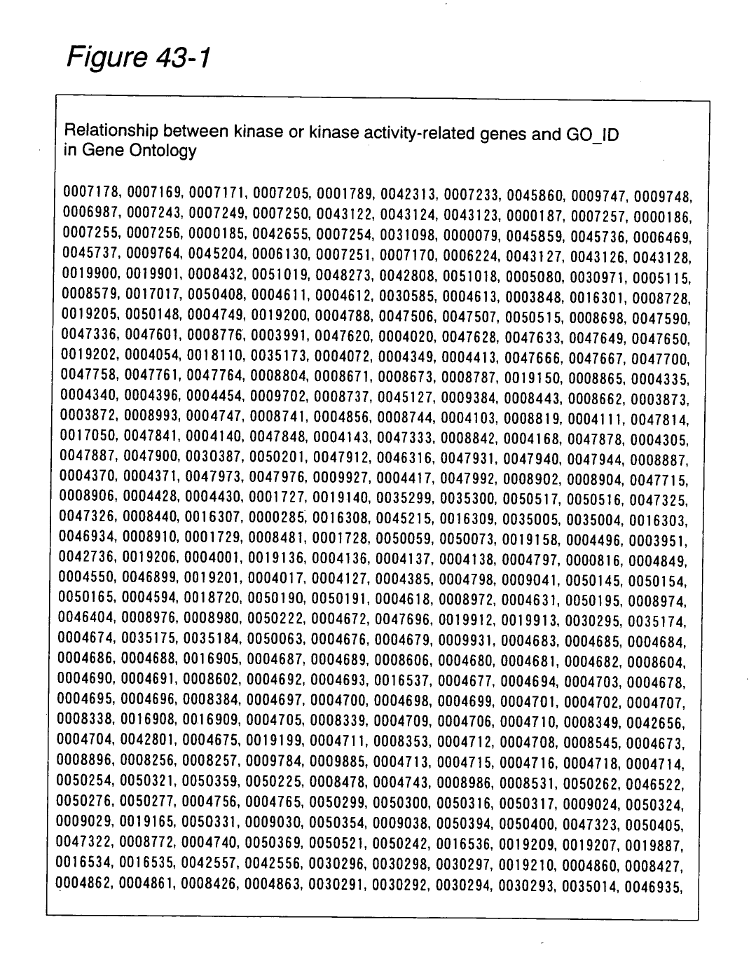

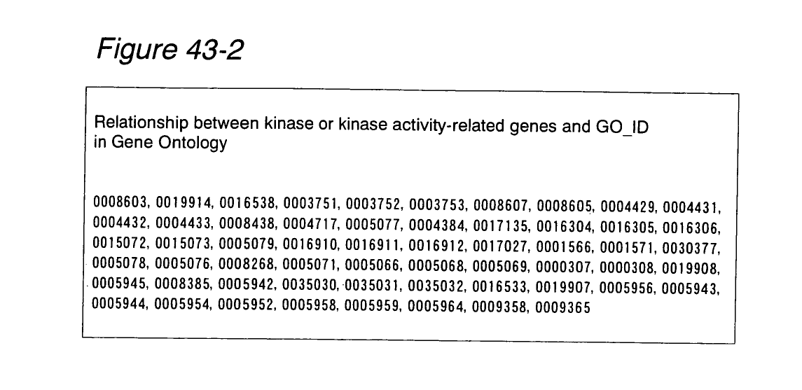

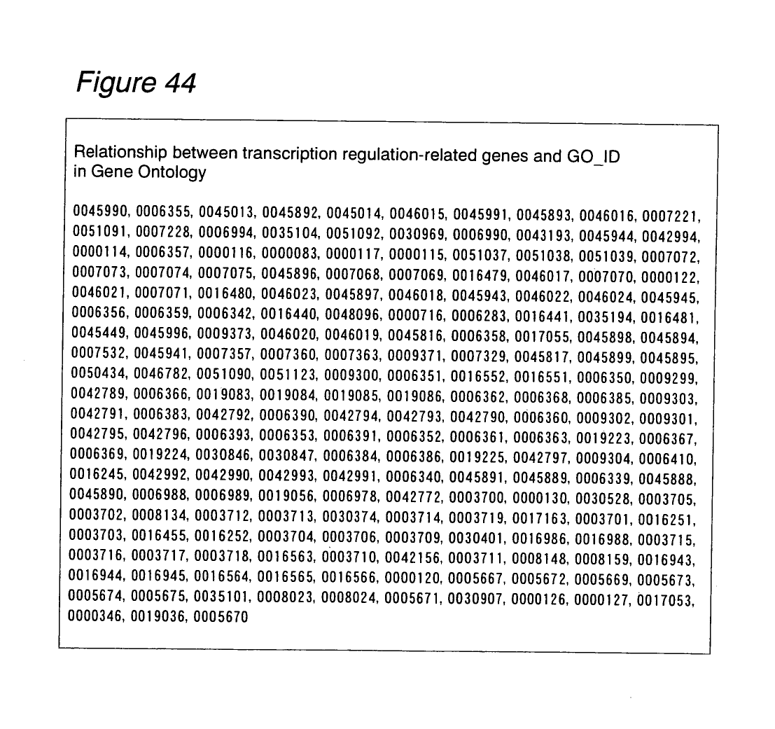

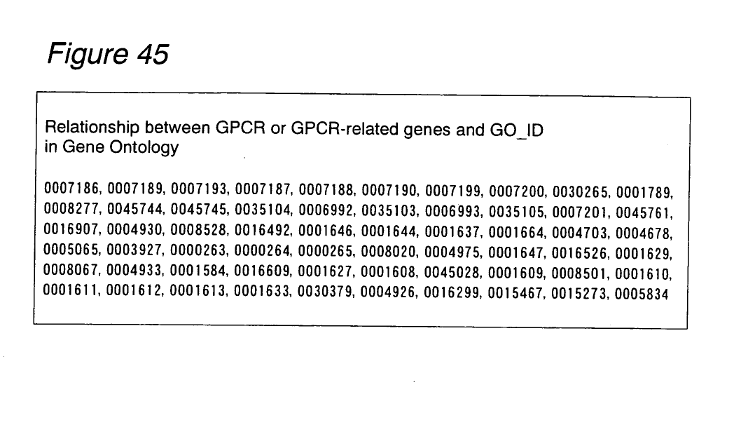

The column “Biological Function Category” of FIG. 46 shows biological functions classified into the above 9 categories. To give more detailed information about what biological function is provided by genes belonging to each group of 1) to 9) above, the relationship with GO_ID in Gene Ontology is shown for each gene in FIGS. 37 to 45. The 7-digit numbers shown in FIGS. 37 to 45 each denote an attribute (more specifically an ID number) in Gene Ontology belonging to each group.

For details about Gene Ontology, refer to, e.g., the Gene Ontology Consortium, “Gene Ontology Consortium home page,” [online], 1999, the Gene Ontology Consortium, [searched on Oct. 25, 2004], Internet <URL: http://www.geneontology.org/>.

For example, Gene Ontology defines gene attributes such as “signal transducer activity (GO:0004871)” and “receptor activity (GO:0004872)” and further defines inherited relationships between attributes to describe, e.g., that “the attribute of receptor activity inherits the attribute of signal transducer activity.” The definitions of attributes and inherited relationships between attributes are available from the Gene Ontology Consortium (http://www.geneontology.org/). Likewise, corresponding relationships between individual human or mouse genes and Gene Ontology attributes are available from various databases including the Cancer genome Anatomy project (http://cgap.nci.nih.gov/). Gene Ontology data of genes, for example, indicate that the human ZYX gene (NM_{—}003461) has receptor activity and further lead to the fact that the ZYX gene also has signal transducer activity when using inherited relationships between attributes.

With respect to gene attributes (annotations), Gene Ontology provides a definition for each attribute and defines inherited relationships between attributes. These inherited relationships between attributes in the ontology of genes form directed acyclic graphs (DAGs). In Gene Ontology, genes are classified and organized by “molecular function”, “biological process” and “cellular component.” Moreover, each classification defines inherited relationships between attributes. Once the ID numbers of attributes in Gene Ontology have been identified, those skilled in the art will understand the details of each attribute from its ID number.

In addition to the above 9 biological function categories according to Gene Ontology, FIG. 46 shows biological functional information of each gene, which is obtained from the reported documents. More specifically, biological functional information of each gene reported in the documents obtained from PubMed is shown in the column “Reported Biological Function.”

In a more preferred embodiment, the pharmaceutical composition of the present invention more preferably comprises a polynucleotide targeting the base sequence shown in any of SEQ ID NOs listed in the column “SEQ ID NO (human)” or “SEQ ID NO (mouse)” of FIG. 46. Each polynucleotide is useful in treating and/or preventing a disease shown in the column “Related Disease” under the same reference number as that of its target sequence. Alternatively, each polynucleotide is useful in controlling a biological function(s) (e.g., inhibition and promotion) shown in the column “Biological Function Category” or “Reported Biological Function” under the same reference number, or in treating and/or preventing a disease(s) associated with the biological function(s).

Table 1 in Example 8 described herein later shows the polynucleotides of the present invention, more specifically, siRNA sense strands corresponding to these polynucleotides (whose base sequences are shown in the column “siRNA-sense” of Table 1), their antisense strands (whose base sequences are shown in the column “siRNA-antisense” of Table 1, provided that the sequences are shown in the direction from 3′ to 5′), target genes to be targeted by these siRNA sequences for RNAi (which are shown in the column “Gene Name” of Table 1) and the positions of target sequences in these genes. As shown in Table 1, the polynucleotides of the present invention served as siRNA-sense or siRNA-antisense strands to produce an RNAi effect against the genes listed in the column “Gene Name” of Table 1, thereby significantly inhibiting the expression of these genes. Thus, pharmaceutical compositions comprising the polynucleotides of the present invention are useful in treating or preventing diseases related to the genes listed in the column “Gene Name” of Table 1, more specifically, diseases corresponding to the genes, as listed in the column “Related Disease” of FIG. 46, as well as diseases associated with biological functions corresponding to the genes, as listed in the columns “Biological Function Category” and/or “Reported Biological Function” of FIG. 46.

In Example 8, the sequences used as targets of siRNA (see the column “Target Sequence” of Table 1) were selected at random from the 53998 target sequences shown in FIG. 46 among possible target sequences to be targeted by the polynucleotides of the present invention. As described later, all the selected target sequences were confirmed to have an RNAi effect. When the results thus obtained in Example 8 were statistically processed by the “population ratio estimation method,” it was found to be statistically reasonable that the polynucleotides of the present invention (more specifically, polynucleotides whose one strand in the double-stranded region is a sequence homologous to a prescribed sequence of a target gene shown in any of SEQ ID NOs: 47 to 817081) would produce an inhibitory effect on the expression of target genes, and that particularly when using polynucleotides in which the above prescribed sequence is any of the 53998 sequences shown in FIG. 46, almost all of them would produce an inhibitory effect on the expression of target genes.

Genes to be targeted by the polynucleotides of the present invention may be those related to any of the diseases shown in FIG. 46. Particularly when the target genes are those related to various cancers including bladder cancer, breast cancer, colorectal cancer, gastric cancer, hepatoma, lung cancer, melanoma, ovarian cancer, pancreas cancer, prostate cancer, oral cancer, skin cancer and thyroid gland cancer, it becomes possible to treat or prevent these various cancers through an inhibitory effect on the expression of the genes. Thus, without being limited thereto, the pharmaceutical composition of the present invention is useful in treating or preventing any cancer selected from those listed above.

The pharmaceutical composition of the present invention most preferably comprises a polynucleotide having any of the base sequences shown in SEQ ID NOs: 817102 to 817651. Each polynucleotide can inhibit the expression of its target gene (see the column “Gene Name” of Table 1) and hence is useful in treating and/or preventing a disease related to the gene (more specifically, see the column “Related Disease” of FIG. 46 with respect to the gene) or a disease associated with a biological function(s) of the gene (more specifically, see the columns “Biological Function Category” and/or “Reported Biological Function” of FIG. 46 with respect to the gene). It is also possible to use sequences having mutations (e.g., mismatches as described above) in the base sequences of these SEQ ID NOs, as long as their RNAi effect is not impaired.

In Examples 1 to 8 described later, a large number of polynucleotides selected according to the selection method of the present invention were demonstrated to produce a significant RNAi effect. Thus, those skilled in the art will easily understand that polynucleotides selected according to common rules produce the same RNAi effect. Moreover, the validity of these rules is also evident from the above statistically processed results. It is therefore easily understood that in the genes shown in Table 1, for example, when a sequence different from the disclosed target sequence is selected from the same gene according to the present invention from a different position than the actually disclosed target position of the target sequence, the same inhibitory effect on gene expression is obtained for the same gene. Moreover, once an inhibitory effect on the expression of a gene related to a certain disease has been identified, it will be easily understood that when its target sequence is selected according to the present invention to prepare a polynucleotide, treatment and/or prevention of the disease through an inhibitory effect on expression is also possible for other genes related to the same disease.

Moreover, Example 7 of the present invention has shown that even in the case of genes other than those containing a sequence completely homologous to a target sequence, when these other genes contain similar sequences having a small number (preferably 2 or less bases) of mismatches, these similar sequence portions may serve as targets for RNA interference. Thus, such genes containing similar sequences, which are other than those containing a sequence completely homologous to a target sequence, are also used as targets of the polynucleotide of the present invention and are expected to produce an RNA interference-based inhibitory effect on expression. The pharmaceutical composition of the present invention is therefore also useful in treating and/or preventing diseases in which these genes are involved.

In a case where a polynucleotide for causing RNAi is used for a pharmaceutical composition, a pharmaceutically acceptable carrier or diluent and the polynucleotide of the present invention may be blended into a pharmaceutical composition. In this case, the ratio of active ingredient to carrier or diluent ranges from about 0.01% to about 99.9% by weight.

The above carrier or diluent may be in gaseous, liquid or solid form. Examples of the carrier include aqueous or alcohol solutions or suspensions, oil solutions or suspensions, oil-in-water or water-in-oil emulsions, hydrophobic carriers, liquid vehicles, and microcrystals.

Moreover, the pharmaceutical composition of the present invention comprising the above polynucleotide may further comprise, for example, at least one of the following: other therapeutic agents, surfactants, fillers, buffers, dispersants, antioxidants and preservatives. Such a pharmaceutical composition may be a formulation for oral, intraoral, intrapulmonary, intrarectal, intrauterine, intratumoral, intracranial, nasal, intramuscular, subcutaneous, intravascular, intrathecal, percutaneous, intracutaneous, intraarticular, intracavitary, ocular, vaginal, ophthalmic, intravenous, intraglandular, interstitial, intralymphatic, implantable, inhalant or sustained release use, or an enteric-coated formulation.

For example, an oral formulation comprising a polynucleotide may be in a dosage form of powders, sugar-coated pills, tablets, capsules, syrups, aerosols, solutions, suspensions or emulsions (e.g., oil-in-water or water-in-oil emulsions). Alternatively, topical formulations are also acceptable, whose carrier is a cream, a gel, an ointment, a syrup, an aerosol, a patch, a solution, a suspension or an emulsion. Moreover, injectable formulations and percutaneous formulations are also acceptable, whose carrier is an aqueous or alcohol solution or suspension, an oil solution or suspension, or an oil-in-water or water-in-oil emulsion. Further, rectal formulations and suppositories are also acceptable. Furthermore, it is also possible to use formulations provided in the form of implants, capsules or cartridges, as well as respirable or inhalant formulations, and aerosols.

The dose of such a pharmaceutical composition comprising a polynucleotide will be selected as appropriate for the symptoms, age and body weight of a patient, etc. With respect to how to administer the pharmaceutical composition to a recipient, in a case where the recipient is a cell or tissue, administration may be accomplished by using techniques such as the calcium phosphate method, electroporation, lipofection, virus infection, and immersion in a polynucleotide solution. Likewise, when introducing into an embryo, it is possible to use microinjection, electroporation, virus infection, etc. For administration, conventionally used commercially available reagents, instruments, apparatuses, kits and the like may be used. For example, an introducing reagent such as TransIT®-In Vivo Gene Delivery System or TransIT®-QR Hydrodynamic Delivery Solution (both manufactured by Takara Bio Inc., Japan) may be used for administration to cells in living organisms. Likewise, for introduction by virus infection, retrovirus vectors (e.g., RNAi Ready pSIREN-RetroQ Vector, manufactured by BD Biosciences Clontech), adenovirus vectors (e.g., BD Knockout Adenoviral RNAi System, manufactured by BD Biosciences Clontech) or lentivirus vectors (e.g., RetroNectin, manufactured by Takara Bio Inc., Japan) may also be used.

In a case where the recipient is a plant, administration may be accomplished by using techniques for injection or spraying into a cavity or interstitial cells in the plant. Likewise, in a case where the recipient is an animal individual, administration may be accomplished, e.g., by oral, parenteral, transvaginal, transrectal, transnasal, transocular or intraperitoneal route. These techniques allow systemic or topical administration of one or more polynucleotides at the same time or at different times. By way of example for oral administration, a pharmaceutical agent or food incorporated with a polynucleotide(s) may be taken directly. Alternatively, by way of example for oral and transnasal routes, administration may be performed using an inhalator. Likewise, by way of example for parenteral route, syringes with or without needles may be used for, e.g., subcutaneous, intramuscular or intravenous administration.

### <9> Composition for Inhibiting Gene Expression

The present invention further provides a composition for inhibiting gene expression to inhibit the expression of a target gene, which comprises the polynucleotide of the present invention.

As has been shown in the present invention, the polynucleotide of the present invention produces an expression inhibitory effect against a gene containing each target sequence. Inhibited expression of the gene controls, preferably inhibits, biological functions of the gene.

Preferably, the target gene is related to any of the diseases listed in the column “Related Disease” of FIG. 46.

Preferably, the target gene is any of the genes listed in the column “Gene Name” of FIG. 46.

Alternatively, the target gene is a gene belonging to any of the following 1) to 9):

1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or

### 9) G protein-coupled receptor or a G protein-coupled receptor-related gene.

As described in the above section “Pharmaceutical composition,” the polynucleotide of the present invention (more specifically siRNA) has been found to produce an RNAi effect based on the results of Example 8 and their statistically processed results. In particular, in Example 8 described later, the polynucleotide of the present invention was confirmed to produce an inhibitory effect on mRNA expression (i.e., RNAi effect) against all the genes listed in the column “Gene Name” of Table 1. Thus, the composition for inhibiting gene expression, which comprises the polynucleotide of the present invention, may target any of the target genes shown in FIG. 46; the target gene is more preferably any of the genes listed in the column “Gene Name” of Table 1. With respect to each gene in Table 1, it is preferably desirable to use a sequence of siRNA-sense or siRNA-antisense shown in the same line as the gene. It is also possible to use sequences having mutations (e.g., mismatches as described above) in these base sequences, as long as their inhibitory effect on expression is not impaired.

If the target gene is any of the genes shown in Table 1, the composition is useful in treating and/or preventing a disease related to the gene (more specifically, see the column “Related Disease” of FIG. 46 with respect to the gene) or a disease associated with a biological function(s) of the gene (more specifically, see the columns “Biological Function Category” and/or “Reported Biological Function” of FIG. 46 with respect to the gene).

For example, genes to be targeted by the composition for inhibiting gene expression in accordance with the present invention may be those related to any of the diseases shown in FIG. 46. Particularly when the target genes are those related to various cancers including bladder cancer, breast cancer, colorectal cancer, gastric cancer, hepatoma, lung cancer, melanoma, ovarian cancer, pancreas cancer, prostate cancer, oral cancer, skin cancer and thyroid gland cancer, it becomes possible to treat or prevent these various cancers through an inhibitory effect on the expression of the genes. Thus, without being limited thereto, the pharmaceutical composition of the present invention is useful in treating or preventing any cancer selected from those listed above.

In Examples 2 to 5 described herein later, the RNAi effect of the polynucleotide of the present invention against the genes of human vimentin, luciferase, SARS virus and the like was examined as a relative expression level of mRNA compared to the control. FIGS. 31, 32 and 35 show the results of mRNA expression levels measured by quantitative PCR. In FIGS. 31, 32 and 35, the relative mRNA expression levels are respectively reduced to about 7-8% (Example 2, FIG. 31), about 12-13% (Example 3, FIG. 32), and a few % to less than about 15% (Example 5, FIG. 35); the polynucleotide of the present invention was confirmed to have an inhibitory effect on the expression of each gene. Likewise, FIG. 34 from Example 4 shows the results of mRNA expression levels (as RNAi effect) examined by luciferase activity. The luciferase activity was also reduced to a few % to less than about 20%, as compared to the control.

Moreover, in Example 8, among the genes shown in FIG. 46 whose related diseases and/or biological functions have been identified, about 300 genes selected at random were examined for the expression levels of their mRNA in human-derived HeLa cells, expressed as relative expression levels. As shown in Table 1, the RQ values (described later) that were calculated to evaluate an inhibitory effect on the expression of these genes, i.e., an RNAi effect were all less than 1, and almost all less than 0.5.

In the composition for inhibiting gene expression in accordance with the present invention, the phrase “inhibiting the expression of the target gene” means that the mRNA expression level of the target gene is substantially reduced. If the mRNA expression level has been substantially reduced, inhibited expression has been achieved regardless of the degree of change in the mRNA expression level. In light of the results from Examples 2-5 and 8 as described above, the composition for inhibiting gene expression in accordance with the present invention is identified to preferably cause at least a 50% or more reduction in the mRNA expression level of the target gene.

### <10> Method for Treating or Preventing Diseases

The present invention further provides a method for treating or preventing the diseases listed in the column “Related Disease” of FIG. 46, which comprises administering a pharmaceutically effective amount of the polynucleotide of the present invention.

### OTHER EMBODIMENTS

One preferred embodiment of the present invention has been described above. However, it is to be understood that the present invention can be carried out in various embodiments other than the embodiment described above within the scope of the technical idea described in the claims.

For example, although the case in which the base sequence processing apparatus 100 performs processing on a stand-alone mode has been described, construction may be made such that processing is performed in accordance with the request from a client terminal which is constructed separately from the base sequence processing apparatus 100, and the processing results are sent back to the client terminal. Specifically, for example, the client terminal transmits a name of the target gene for RNA interference (e.g., gene name or accession number) or base sequence information regarding the target gene to the base sequence processing apparatus 100, and the base sequence processing apparatus 100 performs the processes described above in the controller 102 on base sequence information corresponding to the name or the base sequence information transmitted from the client terminal to select prescribed sequence information which specifically causes RNA interference in the target gene and transmits it to the client terminal. In such a case, for example, by acquiring sequence information from a public database, siRNA against the gene in query may be selected. Alternatively, for example, siRNA for all the genes may be calculated and stored preliminarily, and siRNA may be immediately selected in response to the request from the client terminal (e.g., gene name or accession number) and the selected siRNA may be sent back to the client terminal.

Furthermore, the base sequence processing apparatus 100 may check the specificity of prescribed sequence information with respect to genes unrelated to the target gene. Thereby, it is possible to select prescribed sequence information which specifically causes RNA interference only in the target gene.

Furthermore, in the system comprising a client terminal and the base sequence processing apparatus 100, an interface function may be introduced in which, for example, the results of RNA interference effect of siRNA (e.g., “effective” or “not effective”) are fed back from the Web page users on the Web, and the experimental results fed back from the users are accumulated in the base sequence processing apparatus 100 so that the sequence regularity of siRNA effective for RNA interference is improved.

Furthermore, the base sequence processing apparatus 100 may calculate base sequence information of a sense strand of siRNA and base sequence information of an antisense strand complementary to the sense strand from the prescribed sequence information. Specifically, for example, when “caccctgacccgcttcgtcatgg” is selected as 23-base sequence information wherein 2-base overhanging portions are added to both ends of the prescribed sequence as a result of the processes described above, the base sequence processing apparatus 100 calculates the base sequence information of a sense strand “5′-CCCUGACCCGCUUCGUCAUGG-3” and the base sequence information of an antisense strand “5′-AUGACGAAGCGGGUCAGGGUG-3”. Consequently, it is not necessary to manually arrange the sense strand and the antisense strand when a polynucleotide is ordered, thus improving convenience.

Furthermore, in the processes described in the embodiment, the processes described as being automatically performed may be entirely or partially performed manually, or the processes described as being manually performed may be entirely or partially performed automatically by a known method.

In addition, processing procedures, control procedures, specific names, information including various registration data and parameters, such as search conditions, examples of display screen, and database structures may be changed in any manner except when otherwise described.

Furthermore, with respect to the base sequence processing apparatus 100, the components are shown in the drawings only based on the functional concept, and it is not always necessary to physically construct the components as shown in the drawings.

For example, the process functions of the individual parts or individual units of the base sequence processing apparatus 100, in particular, the process functions performed in the controller 102, may be entirely or partially carried out by a CPU (Central Processing Unit) or programs which are interpreted and executed by the CPU. Alternatively, it may be possible to realize the functions based on hardware according to a wired logic. Additionally, the program is recorded in a recording medium which will be described below and is mechanically read by the base sequence processing apparatus 100 as required.

Namely, the memory 106, such as a ROM or HD, records a computer program which, together with OS (Operating System), gives orders to the CPU to perform various types of processing. The computer program is executed by being loaded into a RAM or the like, and, together with the CPU, constitutes the controller 102. Furthermore, the computer program may be recorded in an application program server which is connected to the base sequence processing apparatus 100 via any network 300, and may be entirely or partially downloaded as required.

The program of the present invention may be stored in a computer-readable recording medium. Here, examples of the “recording medium” include any “portable physical medium”, such as a flexible disk, an optomagnetic disk, a ROM, an EPROM, an EEPROM, a CD-ROM, a MO, a DVD, or a flash disk; any “fixed physical medium”, such as a ROM, a RAM, or a HD which is incorporated into various types of computer system; and a “communication medium” which holds the program for a short period of time, such as a communication line or carrier wave, in the case when the program is transmitted via a network, such as a LAN, a WAN, or Internet.

Furthermore, the “program” means a data processing method described in any language or by any description method, and the program may have any format (e.g., source code or binary code). The “program” is not always limited to the one having a single system configuration, and may have a distributed system configuration including a plurality of modules or libraries, or may achieve its function together with another program, such as OS (Operating System). With respect to specific configurations and procedures for reading the recording medium in the individual units shown in the embodiment, or installation procedures after reading, etc., known configurations and procedures may be employed.

The various types of databases, etc. (target gene base sequence file 106a˜target gene annotation database 106h) stored in the memory 106 are storage means, such as memories (e.g., RAMs and ROMs), fixed disk drives (e.g., hard disks), flexible disks, and optical disks, which store various types of programs used for various processes and Web site provision, tables, files, databases, files for Web pages, etc.

Furthermore, the base sequence processing apparatus 100 may be produced by connecting peripheral apparatuses, such as a printer, a monitor, and an image scanner, to a known information processing apparatus, for example, an information processing terminal, such as a personal computer or a workstation, and installing software (including programs, data, etc.) which implements the method of the present invention into the information processing apparatus.

Furthermore, specific modes of distribution/integration of the base sequence processing apparatus 100, etc. are not limited to those shown in the specification and the drawings, and the base sequence processing apparatus 100, etc., may be entirely or partially distributed/integrated functionally or physically in any unit corresponding to various types of loading, etc. (e.g., grid computing). For example, the individual databases may be independently constructed as independent database units, or processing may be partially performed using CGI (Common Gateway Interface).

Furthermore, the network 300 has a function of interconnecting between the base sequence processing apparatus 100 and the external system 200, and for example, may include any one of the Internet, intranets, LANs (including both wired and radio), VANs, personal computer communication networks, public telephone networks (including both analog and digital), dedicated line networks (including both analog and digital), CATV networks, portable line exchange networks/portable packet exchange networks of the IMT2000 system, CSM system, or PDC/PDC-P system, radio paging networks, local radio networks, such as the Bluetooth, PHS networks, and satellite communication networks, such as CS, BS, and ISDB. Namely, the present system can transmit and receive various types of data via any network regardless of wired or radio.

### EXAMPLES

The present invention will be described in more detail with reference to the examples. However, it is to be understood that the present invention is not restricted by the examples.

### Example 1

### <1> Gene for Measuring RNAi Effect and Expression Vector

As a target gene for measuring an RNAi effect by siRNA, a firefly (Photinus pyralis, P. pyralis) luciferase (luc) gene (P. pyralis luc gene: accession number: U47296) was used, and as an expression vector containing this gene, a pGL3-Control Vector (manufactured by Promega Corporation) was used. The segment of the P. pyralis luc gene is located between an SV40 promoter and a poly A signal within the vector. As an internal control gene, a luc-gene of sea pansy (Renilla reniformis, R. reniformis) was used, and as an expression vector containing this gene, pRL-TK (manufactured by Promega Corporation) was used.

### <2> Synthesis of 21-Base Double-Stranded RNA (siRNA)

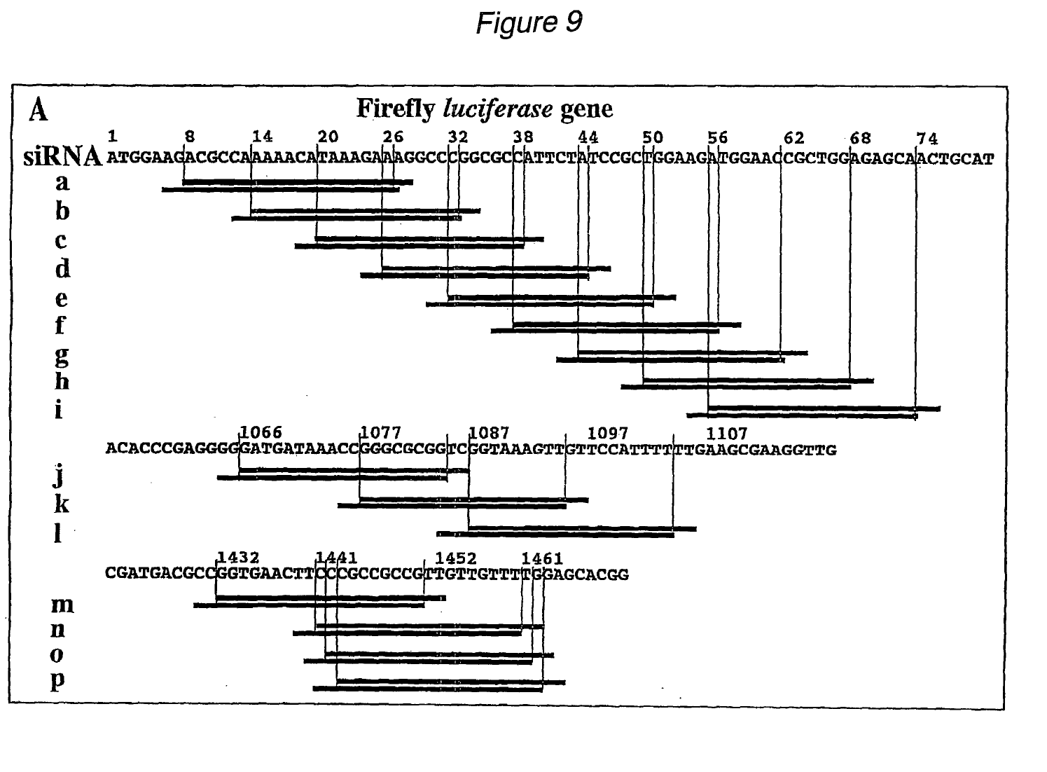

Synthesis of 21-base sense strand and 21-base antisense strand RNA (located as shown in FIG. 9; a to p) was entrusted to Genset Corporation through Hitachi Instrument Service Co., Ltd.

The double-stranded RNA used for inhibiting expression of the P. pyralis luc gene was prepared by associating sense and antisense strands. In the association process, the sense strand RNA and the antisense strand RNA were heated for 3 minutes in a reaction liquid of 10 mM Tris-HCl (pH 7.5) and 20 mM NaCl, incubated for one hour at 37° C., and left to stand until the temperature reached room temperature. Formation of double-stranded polynucleotides was assayed by electrophoresis on 2% agarose gel in a TBE buffer, and it was confirmed that almost all the single-stranded polynucleotides were associated to form double-stranded polynucleotides.

### <3> Mammalian Cell Cultivation

As mammalian cultured cells, human HeLa cells and HEK293 cells and Chinese hamster CHO-KI cells (RIKEN Cell bank) were used. As a medium, Dulbecco's modified Eagle's medium (manufactured by Gibco BRL) to which a 10% inactivated fetal bovine serum (manufactured by Mitsubishi Kasei) and as antibiotics, 10 units/ml of penicillin (manufactured by Meiji) and 50 μg/ml of streptomycin (manufactured by Meiji) had been added was used. Cultivation was performed at 37° C. in the presence of 5% CO₂.

### <4> Transfection of Target Gene, Internal Control Gene, and siRNA into Mammalian Cultured Cells

The mammalian cells were seeded at a concentration of 0.2 to 0.3×10⁶ cells/ml into a 24-well plate, and after one day, using a Ca-phosphate precipitation method (Saibo-Kogaku Handbook (Handbook for cell engineering), edited by Toshio Kuroki et al., Yodosha (1992)), 1.0 μg of pGL3-Control DNA, 0.5 or 1.0 μg of pRL-TK DNA, and 0.01, 0.1, 1, 10 or 100 nM of siRNA were introduced.

### <5> Drosophila Cell Cultivation

As drosophila cultured cells, S2 cells (Schneider, I., et al., J. Embryol. Exp. Morph., 27, 353-365 (1972)) were used. As a medium, Schneider's Drosophila medium (manufactured by Gibco BRL) to which a 10% inactivated fetal bovine serum (manufactured by Mitsubishi Kasei) and as antibiotics, 10 units/ml of penicillin (manufactured by Meiji) and 50 μg/ml of streptomycin (manufactured by Meiji) had been added was used. Cultivation was performed at 25° C. in the presence of 5% CO₂.

### <6> Transfection of Target Gene, Internal Control Gene, and siRNA into Drosophila Cultured Cells

The S2 cells were seeded at a concentration of 1.0×10⁶ cells/ml into a 24-well plate, and after one day, using a Ca-phosphate precipitation method (Saibo-Kogaku Handbook (Handbook for cell engineering), edited by Toshio Kuroki et al., Yodosha (1992)), 1.0 μg of pGL3-Control DNA, 0.1 μg of pRL-TK DNA, and 0.01, 0.1, 1, 10 or 100 nM of siRNA were introduced.

### <7> Measurement of RNAi Effect

The cells transfected with siRNA were recovered 20 hours after transfection, and using a Dual-Luciferase Reporter Assay System (manufactured by Promega Corporation), the levels of expression (luciferase activities) of two types of luciferase (P. pyralis luc and reniformis luc) protein were measured. The amount of luminescence was measured using a Lumat LB9507 luminometer (EG&G Berthold).

### <8> Results

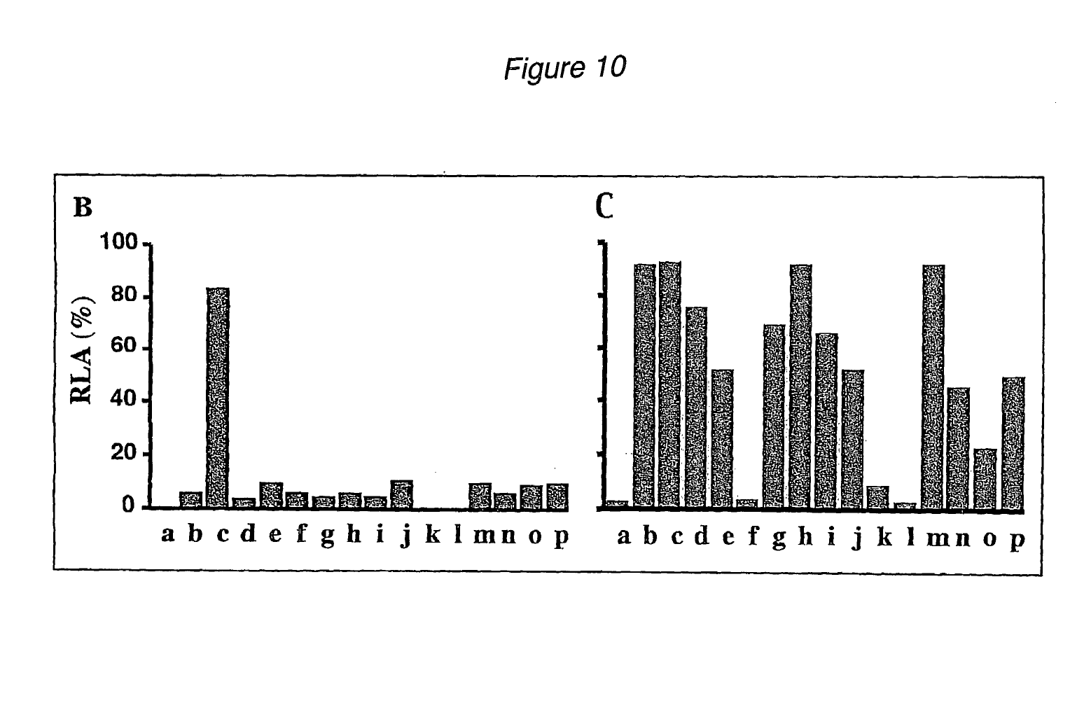

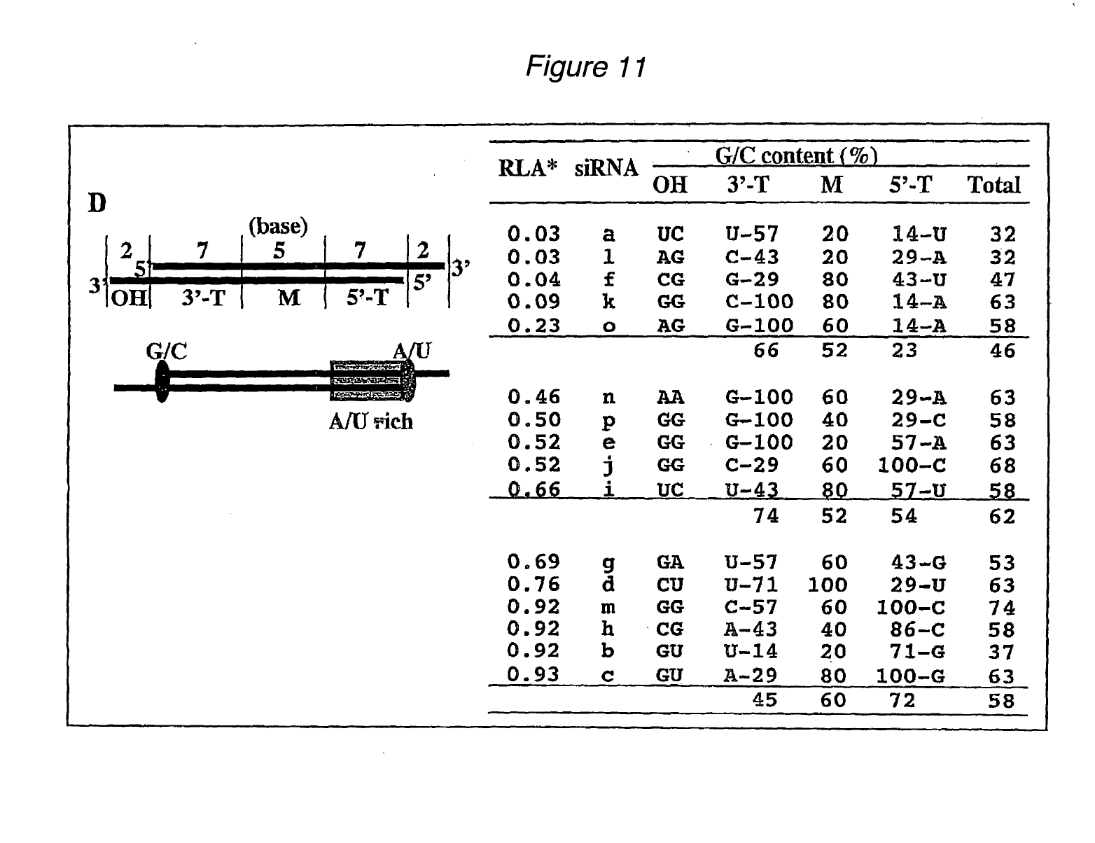

The measurement results on the luciferase activities are shown in FIG. 10. Furthermore, the results of study on correspondence between the luciferase activities and the individual base sequences are shown in FIG. 11.

In FIG. 10, the graph represented by B shows the results in the drosophila cells, and the graph represented by C shows the results in the human cells. As shown in FIG. 10, in the drosophila cells, by creating RNA with a base number of 21, it was possible to inhibit the luciferase activities in almost all the sequences. On the other hand, in the human cells, it was evident that it was difficult to obtain sequences which could inhibit the luciferase activities simply by setting the base number at 21.

Analysis was then conducted on the regularity of base sequence with respect to RNA a to p. As shown in FIG. 11, with respect to 5 points of the double-stranded RNA, the base sequence was analyzed. With respect to siRNA a in the top row of the table shown in FIG. 11, the relative luciferase activity (RLA) is 0.03. In the antisense strand, from the 3′ end, the base sequence of the overhanging portion (OH) is UC; the G/C content (content of guanine or cytosine) in the subsequent 7 bases (3′-T in FIG. 11) is 57%; the G/C content in the further subsequent 5 bases (M in FIG. 11) is 20; the G/C content in the further subsequent 7 bases (5′-T in FIG. 11) is 14%; the 5′ end is U; and the G/C content in total is 32%. In the table, a lower RLA value indicates lower RLA activity, i.e., inhibition of the expression of luciferase.

As is evident from the results, in the base sequences of polynucleotides for causing RNA interference, it is highly probable that the 3′ end is adenine or uracil and that the 5′ end is guanine or cytosine. Furthermore, it has become clear that the 7-base sequence from the 3′ end is rich in adenine or uracil.

### Example 2

### 1. Construction of Target Expression Vector pTREC

A target expression vector was constructed as follows. A target expression molecule is a molecule which allows expression of RNA having a sequence to be targeted by RNAi (hereinafter, also referred to as a “target sequence”).

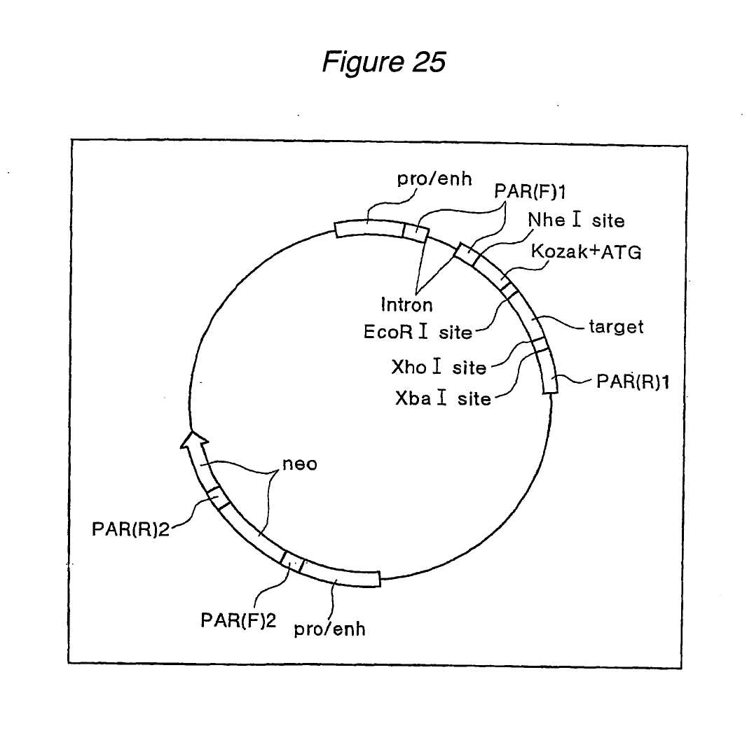

A target mRNA sequence was constructed downstream of the CMV enhancer/promoter of pCI-neo (GenBank Accession No. U47120, manufactured by Promega Corporation) (FIG. 25). That is, the following double-stranded oligomer was synthesized, the oligomer including a Kozak sequence (Kozak), an ATG sequence, a cloning site having a 23 base-pair sequence to be targeted (target), and an identification sequence for restriction enzyme (NheI, EcoRI, XhoI) for recombination. The double-stranded oligomer consists of a sequence shown in SEQ ID NO: 1 in the sequence listing and its complementary sequence. The synthesized double-stranded oligomer was inserted into the NheI/XbaI site of the pCI-neo to construct a target expression vector pTREC (FIG. 25). With respect to the intron, the intron site derived from P-globin originally incorporated in the pCI-neo was used.

5′-gctagccaccatggaattcacgcgtctcgagtctaga-3′ (SEQ ID NO: 1)

The pTREC shown in FIG. 25 is provided with a promoter and an enhancer (pro/enh) and regions PAR(F) 1 and PAR(R) 1 corresponding to the PCR primers. An intron (Intron) is inserted into PAR(F) 1, and the expression vector is designed such that the expression vector itself does not become a template of PCR. After transcription of RNA, in an environment in which splicing is performed in eukaryotic cultured cells or the like, the intron site of the pTREC is removed to join two neighboring PAR(F) 1's. RNA produced from the pTREC can be amplified by RT-PCR. With respect to the intron, the intron site derived from β-globin originally incorporated in the pCI-neo was used.

The pTREC is incorporated with a neomycin-resistant gene (neo) as a control, and by preparing PCR primers corresponding to a part of the sequence in the neomycin-resistant gene and by subjecting the part of the neomycin-resistant gene to RT-PCR, the neomycin-resistant gene can be used as an internal standard control (internal control). PAR(F) 2 and PAR(R) 2 represent the regions corresponding to the PCR primers in the neomycin-resistant gene. Although not shown in the example of FIG. 25, an intron may be inserted into at least one of PAR(F) 2 and PAR(R) 2.

### 2. Effect of Primer for Detecting Target mRNA

### (1) Transfection into Cultured Cells

HeLa cells were seeded at 0.2 to 0.3×10⁶ cells per well of a 24-well plate, and after one day, using Lipofectamine 2000 (manufactured by Invitrogen Corp.), 0.5 μg of pTREC vector was transfected according to the manual.

### (2) Recovery of Cells and Quantification of mRNA

One day after the transfection, the cells were recovered and total RNA was extracted with Trizol (manufactured by Invitrogen Corp.). One hundred nanograms of the resulting RNA was reverse transcribed by SuperScript II RT (manufactured by Invitrogen Corp.), using oligo (dT) primers, to synthesize cDNA. A control to which no reverse transcriptase was added was prepared. Using one three hundred and twentieth of the amount of the resulting cDNA as a PCR template, quantitative PCR was carried out in a 50-μl reaction system using SYBR Green PCR Master Mix (manufactured by Applied Biosystems Corp.) to quantify target mRNA (referred to as mRNA (T)) and, as an internal control, mRNA derived from the neomycin-resistant gene in the pTREC (referred to as mRNA (C)). A real-time monitoring apparatus ABI PRIZM7000 (manufactured by Applied Biosystems) was used for the quantitative PCR. A primer pair T (SEQ ID NOs: 2 and 3 in the sequence listing) and a primer pair C (SEQ ID NOs: 4 and 5 in the sequence listing) were used for the quantification of mRNA (T) and mRNA (C), respectively.

### Primer Pair T:

(SEQ ID NO: 2)

aggcactgggcaggtgtc

(SEQ ID NO: 3)

tgctcgaagcattaaccctcacta

### Primer Pair C

(SEQ ID NO: 4)

atcaggatgatctggacgaag

(SEQ ID NO: 5)

ctcttcagcaatatcacgggt

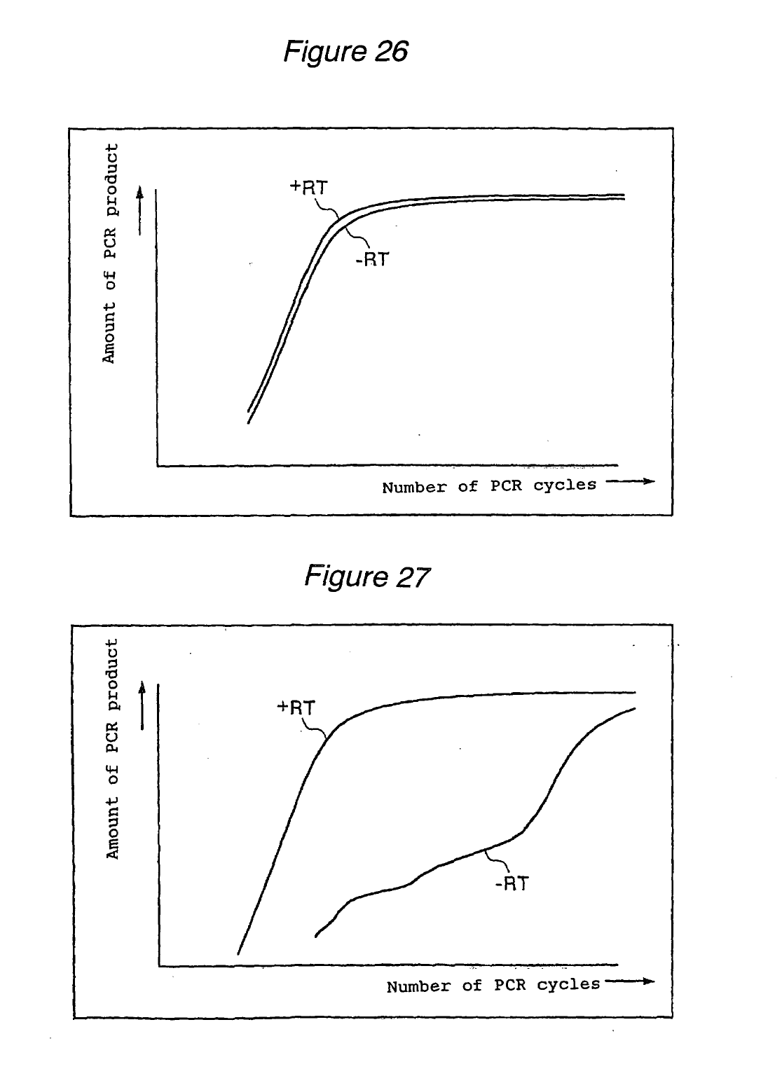

FIGS. 26 and 27 show the results of PCR. Each of FIGS. 26 and 27 is a graph in which the PCR product is taken on the axis of ordinate and the number of cycles of PCR is taken on the axis of abscissa. In the neomycin-resistant gene, there is a small difference in the amplification of the PCR product between the case in which cDNA was synthesized by the reverse transcriptase (+RT) and the control case which no reverse transcriptase was added (−RT) (FIG. 26). This indicates that not only cDNA but also the vector remaining in the cells also acted as a template and was amplified. On the other hand, in target sequence mRNA, there is a large difference between the case in which the reverse transcriptase was added (+RT) and the case in which no transcriptase was added (−RT) (FIG. 27). This result indicates that since one member of the primer pair T is designed so as to sandwich the intron, cDNA derived from intron-free mRNA is efficiently amplified, while the remaining vector having the intron does not easily become a template.

### 3. Inhibition of Expression of Target mRNA by siRNA

### (1) Cloning of Evaluation Sequence to Target Expression Vector

Sequences corresponding to the coding regions 812-834 and 35-57 of a human vimentin (VIM) gene (RefSeq ID: NM_{—}003380) were targeted for evaluation. The following synthetic oligonucleotides (evaluation sequence fragments) of SEQ ID NOs: 6 and 7 in the sequence listing were produced, the synthetic oligonucleotides including these sequences and identification sequences for EcoRI and XhoI.

### Evaluation Sequence VIM35 (Corresponding to 35-57 of VIM)

(SEQ ID NO: 6)

5′-gaattcgcaggatgttcggcggcccgggcctcgag-3′

### Evaluation Sequence VIM812 (Corresponding to 812-834 of VIM)

(SEQ ID NO: 7)

5′-gaattcacgtacgtcagcaatatgaaagtctcgag-3′

Using the EcoRI and XhoI sites located on both ends of each of the evaluation sequence fragments, each fragment was cloned as a new target sequence between the EcoRI and XhoI sites of the pTREC, and thereby pTREC-VIM35 and pTREC-VIM812 were constructed.

### (2) Production of siRNA

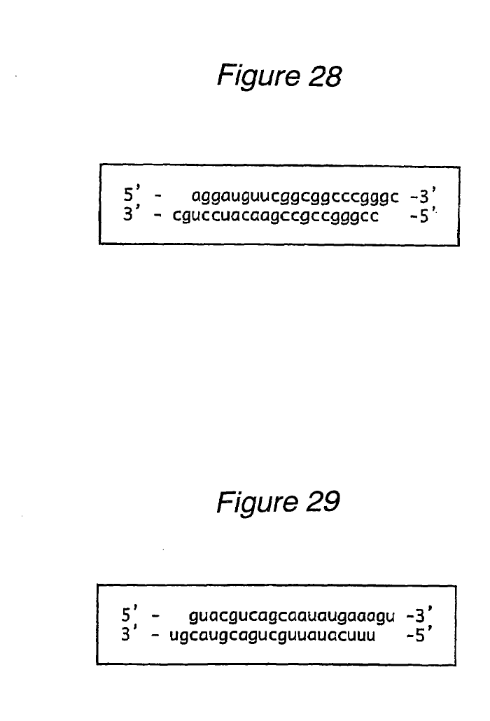

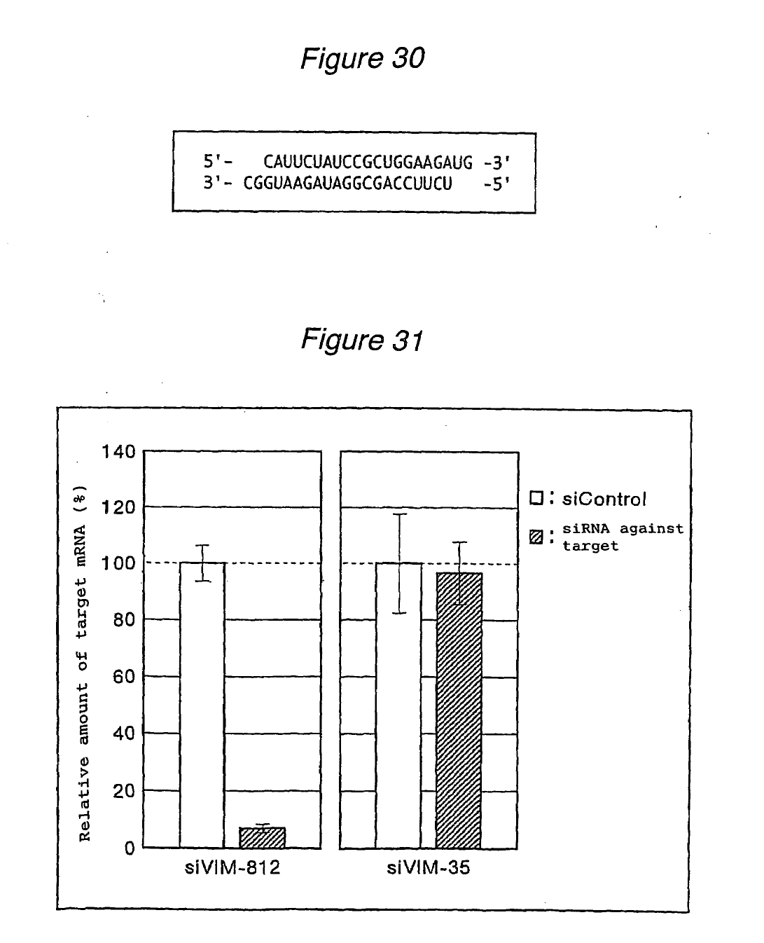

siRNA fragments corresponding to the evaluation sequence VIM35 (SEQ ID NO: 8 in the sequence list, FIG. 28), the evaluation sequence VIM812 (SEQ ID NO: 9, FIG. 29), and a control sequence (siContorol, SEQ ID NO: 10, FIG. 30) were synthesized, followed by annealing. Each of the following siRNA sequences is provided with an overhanging portion on the 3′ end.

siVIM35

(SEQ ID NO: 8)

5′-aggauguucggcggcccgggc-3′
siVIM812

(SEQ ID NO: 9)

5′-guacgucagcaauaugaaagu-3′

As a control, siRNA for the luciferase gene was used.

siControl

(SEQ ID NO: 10)

5′-cauucuauccgcuggaagaug-3′

### (3) Transfection into Cultured Cells

HeLa cells were seeded at 0.2 to 0.3×10⁶ cells per well of a 24-well plate, and after one day, using Lipofectamine 2000 (manufactured by Invitrogen Corp.), 0.5 μg of pTREC-VIM35 or pTREC-VIM812, and 100 nM of siRNA corresponding to the sequence derived from each VIM (siVIM35, siVIM812) were simultaneously transfected according to the manual. Into the control cells, 0.5 μg of pTREC-VIM35 or pTREC-VIM812 and 100 nM of siRNA for the luciferase gene (siControl) were simultaneously transfected.

### (4) Recovery of Cells and Quantification of mRNA

One day after the transfection, the cells were recovered and total RNA was extracted with Trizol (Invitrogen). One hundred nanograms of the resulting RNA was reverse transcribed by SuperScript II RT (manufactured by Invitrogen Corp.), using oligo (dT) primers, to synthesize cDNA. Using one three hundred and twentieth of the amount of the resulting cDNA as a PCR template, quantitative PCR was carried out in a 50-μl reaction system using SYBR Green PCR Master Mix (manufactured by Applied Biosystems Corp.) to quantify mRNA (referred to as mRNA (T)) including the sequence derived from VIM to be evaluated and, as an internal control, mRNA derived from the neomycin-resistant gene in the pTREC (referred to as mRNA (C)).

A real-time monitoring apparatus ABI PRIZM7000 (manufactured by Applied Biosystems) was used for the quantitative PCR. The primer pair T (SEQ ID NOs: 2 and 3 in the sequence listing) and the primer pair C (SEQ ID NOs: 4 and 5 in the sequence listing) were used for the quantification of mRNA (T) and mRNA (C), respectively. The ratio (T/C) of the resulting values of mRNA was taken on the axis of ordinate (relative amount of target mRNA (%)) in a graph (FIG. 31).

In the control cells, since siRNA for the luciferase gene does not affect target mRNA, the ratio T/C is substantially 1. In VIM812 siRNA, the ratio T/C is extremely decreased. The reason for this is that VIM812 siRNA cut mRNA having the corresponding sequence, and it was shown that VIM812 siRNA has the RNAi effect. On the other hand, in VIM35 siRNA, the T/C ratio was substantially the same as that of the control, and thus it was shown that the sequence of VIM35 does not substantially have the RNAi effect.

### Example 3

### 1. Inhibition of Expression of Endogenous Vimentin by siRNA

### (1) Transfection into Cultured Cells

HeLa cells were seeded at 0.2 to 0.3×10⁶ cells per well of a 24-well plate, and after one day, using Lipofectamine 2000 (manufactured by Invitrogen Corp.), 100 nM of siRNA for VIM (siVIM35 or siVIM812) or control siRNA (siControl) and, as a control for transfection efficiency, 0.5 μg of pEGFP (manufactured by Clontech) were simultaneously transfected according to the manual. pEGFP is incorporated with EGFP.

### (2) Assay of Endogenous Vimentin mRNA

Three days after the transfection, the cells were recovered and total RNA was extracted with Trizol (manufactured by Invitrogen Corp.). One hundred nanograms of the resulting RNA was reverse transcribed by SuperScript II RT (manufactured by Invitrogen Corp.), using oligo (dT) primers, to synthesize cDNA. PCR was carried out using the cDNA product as a template and using primers for vimentin, VIM-F3-84 and VIM-R3-274 (SEQ ID NOs: 11 and 12).

VIM-F3-84;

(SEQ ID NO: 11)

gagctacgtgactacgtcca
VIM-R3-274;

(SEQ ID NO: 12)

gttcttgaactcggtgttgat

Furthermore, as a control, PCR was carried out using β-actin primers ACTB-F2-481 and ACTB-R2-664 (SEQ ID NOs: 13 and 14). The level of expression of vimentin was evaluated under the common quantitative value of β-actin for each sample.

ACTB-F2-481;

(SEQ ID NO: 13)

cacactgtgcccatctacga
ACTB-R2-664;

(SEQ ID NO: 14)

gccatctcttgctcgaagtc

The results are shown in FIG. 32. In FIG. 32, the case in which siControl (i.e., the sequence unrelated to the target) is incorporated is considered as 100% for comparison, and the degree of decrease in mRNA of VIM when siRNA is incorporated into VIM is shown. siVIM-812 was able to effectively inhibit VIM mRNA. In contrast, use of siVIM-35 did not substantially exhibit the RNAi effect.

### (3) Antibody Staining of Cells

Three days after the transfection, the cells were fixed with 3.7% formaldehyde, and blocking was performed in accordance with a conventional method. Subsequently, a rabbit anti-vimentin antibody (α-VIM) or, as an internal control, a rabbit anti-Yes antibody (α-Yes) was added thereto, and reaction was carried out at room temperature. Subsequently, the surfaces of the cells were washed with PBS (Phosphate Buffered Saline), and as a secondary antibody, a fluorescently-labeled anti-rabbit IgG antibody was added thereto. Reaction was carried out at room temperature. After the surfaces of the cells were washed with PBS, observation was performed using a fluorescence microscope.

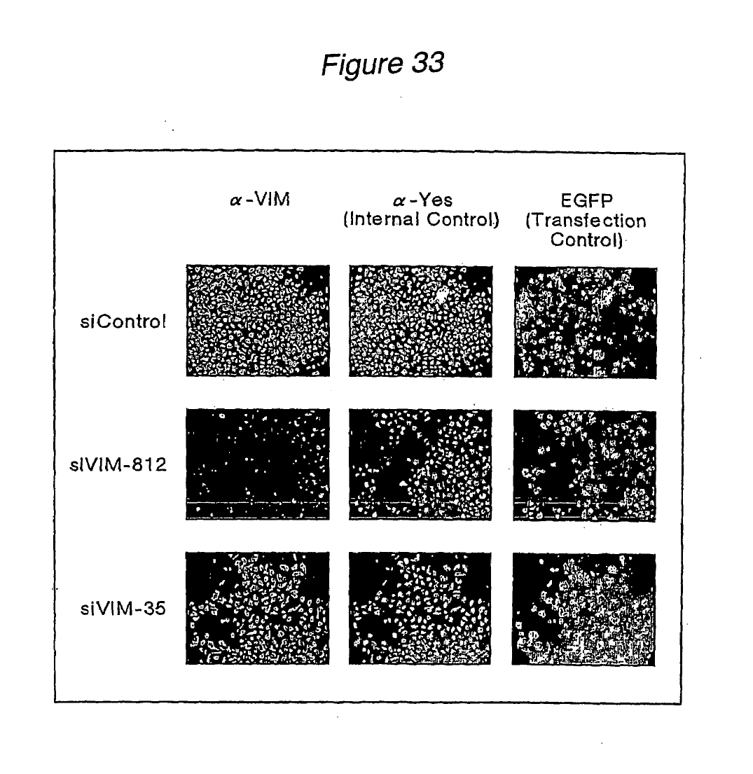

The fluorescence microscope observation results are shown in FIG. 33. In the nine frames of FIG. 33, the parts appearing white correspond to fluorescent portions. In EGFP and Yes, substantially the same expression was confirmed in all the cells. In the cells into which siControl and siVIM35 were introduced, fluorescence due to antibody staining of vimentin was observed, and the presence of endogenous vimentin was confirmed. On the other hand, in the cells into which siVIM812 was introduced, fluorescence was significantly weaker than that of the cells into which siControl and siVIM35 were introduced. The results show that endogenous vimentin mRNA was interfered by siVIM812, and consequently, the level of expression of vimentin protein was decreased. It has become evident that siVIM812 also has the RNAi effect against endogenous vimentin mRNA.

The results obtained in the assay system of the present invention [Example 2] matched well with the results obtained in the cases in which endogenous genes were actually treated with corresponding siRNA [Example 3]. Consequently, it has been confirmed that the assay system is effective as a method for evaluating the RNAi activity of any siRNA.

### Example 4

Base sequences were designed based on the above predetermined rules (a) to (d). The base sequences were designed by a base sequence processing apparatus which runs the siRNA sequence design program. As the base sequences, 15 sequences (SEQ ID NOs: 15 to 29) which were expected to have RNAi activity and 5 sequences (SEQ ID NOs: 30 to 34) which were not expected to have RNAi activity were prepared.

RNAi activity was evaluated by measuring the luciferase activity as in Example 1 except that the target sequence and siRNA to be evaluated were prepared based on each of the designed sequences. The results are shown in FIG. 34. A low luciferase relative activity value indicates an effective state, i.e., siRNA provided with RNAi activity. All of the siRNA which was expected to have RNAi activity by the program effectively inhibited the expression of luciferase.

### [Sequences which Exhibited RNAi Activity; Prescribed Sequence Portions, Excluding Overhanging Portions]

| | |
|---|---|
| 5, gacgccaaaaacataaaga | (SEQ ID NO: 15) |
| 184, gttggcagaagctatgaaa | (SEQ ID NO: 16) |
| 272, gtgttgggcgcgttattta | (SEQ ID NO: 17) |
| 309, ccgcgaacgacatttataa | (SEQ ID NO: 18) |
| 428, ccaatcatccaaaaaatta | (SEQ ID NO: 19) |
| 515, cctcccggttttaatgaat | (SEQ ID NO: 20) |
| 658, gcatgccagagatcctatt | (SEQ ID NO: 21) |
| 695, ccggatactgcgattttaa | (SEQ ID NO: 22) |
| 734, ggttttggaatgtttacta | (SEQ ID NO: 23) |
| 774, gatttcgagtcgtcttaat | (SEQ ID NO: 24) |
| 891, gcactctgattgacaaata | (SEQ ID NO: 25) |
| 904, caaatacgatttatctaat | (SEQ ID NO: 26) |
| 1186, gattatgtccggttatgta | (SEQ ID NO: 27) |
| 1306, ccgcctgaagtctctgatt | (SEQ ID NO: 28) |
| 1586, ctcgacgcaagaaaaatca | (SEQ ID NO: 29) |

### [Sequences which Did Not Exhibit RNAi Activity; Prescribed Sequence Portions, Excluding Overhanging Portions]

| | |
|---|---|
| 14, aacataaagaaaggcccgg | (SEQ ID NO: 30) |
| 265, tatgccggtgttgggcgcg | (SEQ ID NO: 31) |
| 295, agttgcagttgcgcccgcg | (SEQ ID NO: 32) |
| 411, acgtgcaaaaaaagctccc | (SEQ ID NO: 33) |
| 1044, ttctgattacacccgaggg | (SEQ ID NO: 34) |

### Example 5

siRNA sequences against SARS virus were designed and examined for their RNAi activity. RNAi activity was evaluated by the same assay as used in Example 2, except that both the target sequence and the sequence to be evaluated were changed.

siRNA sequences were designed on the basis of the genome of SARS virus by using the above siRNA sequence design program, such that the resulting siRNA sequences satisfied a given regularity for 3CL-PRO, RdRp, Spike glycoprotein, Small envelope E protein, Membrane glycoprotein M, Nucleocapsid protein and s2m motif, respectively.

As a result of the assay shown in FIG. 35, 11 siRNA sequences designed to satisfy the regularity were found to effectively inhibit RNA into which the respective corresponding siRNA sequences were incorporated as targets. The case in which siControl (the sequence unrelated to SARS) is incorporated is considered as 100%, and the relative amount of target mRNA when each siRNA of SARS is incorporated is shown. In the case of incorporating each siRNA, target RNA was reduced to around 10% or below; each siRNA was confirmed to have RNAi activity.

### [Designed siRNA Sequences (Prescribed Sequence Portions, Excluding Overhanging Portions)]

siControl;

(SEQ ID NO: 35)

gggcgcggtcggtaaagtt
3CL-PRO; SARS-10754;

(SEQ ID NO: 36)

ggaattgccgtcttagata
3CL-PRO; SARS-10810;

(SEQ ID NO: 37)

gaatggtcgtactatcctt
RdRp; SARS-14841;

(SEQ ID NO: 38)

ccaagtaatcgttaacaat
Spike glycoprotein; SARS-23341;

(SEQ ID NO: 39)

gcttggcgcatatattcta
Spike glycoprotein; SARS-24375;

(SEQ ID NO: 40)

cctttcgcgacttgataaa
Small envelope E protein; SARS-26233;

(SEQ ID NO: 41)

gtgcgtactgctgcaatat
Small envelope E protein; SARS-26288;

(SEQ ID NO: 42)

ctactcgcgtgttaaaaat
Membrane glycoprotein M; SARS-26399;

(SEQ ID NO: 43)

gcagacaacggtactatta
Membrane glycoprotein M; SARS-27024;

(SEQ ID NO: 44)

ccggtagcaacgacaatat
Nucleocapsid protein; SARS-28685;

(SEQ ID NO: 45)

cgtagtcgcggtaattcaa
s2m motif; SARS-29606;

(SEQ ID NO: 46)

gatcgagggtacagtgaat

### Example 6

According to “<5> siRNA sequence design program” and “<7> Base sequence processing apparatus for running siRNA sequence design program, etc.” described above, the following siRNA sequences were designed. Setting conditions for running the program are as shown below.

### (Setting Conditions)

(a) The 3′ end base is adenine, thymine or uracil.
(b) The 5′ end base is guanine or cytosine.
(c) In a 7-base sequence from the 3′ end, 4 or more bases are one or more types of bases selected from the group consisting of adenine, thymine and uracil.
(d) The number of bases is 19.
(e) A sequence in which 10 or more bases of guanine or cytosine are continuously present is not contained.
(f) A similar sequence containing mismatches of 2 or less bases against the prescribed sequence is not contained in the base sequences of genes other than the target gene among all gene sequences of the target organism.

The designed siRNA sequences are shown in the sequence listing under SEQ ID NOs: 47 to 817081. The name of an organism targeted by each of the siRNA sequences shown in the sequence listing under SEQ ID NOs: 47 to 817081 is shown in <213> of the sequence listing. Likewise, the gene name of each target gene for RNAi, the accession of each target gene, and a prescribed sequence-corresponding portion in the base sequence of each target gene are shown in <223> (Other information) of the sequence listing. It should be noted that gene names and accession information in this context correspond to the “RefSeq” database at NCBI (HYPERLINK “http://www.ncbi.nlm.nih.gov/” http://www.ncbi.nlm.nih.gov/), and information of each gene (including the sequence and function of the gene) can be obtained through access to the RefSeq database.

An example will be given of siRNA shown in SEQ ID NO: 47. The target organism is Homo sapiens, the gene name of the target gene is ATBF1, the accession of the target gene is NM_{—}006885.2, and the portion corresponding to the prescribed sequence is composed of 19 bases between bases 908 and 926 in the base sequence of NM_{—}006885.2. Upon access to the RefSeq database, the target gene will be found to be a gene related to AT-binding transcription factor 1.

### Example 7

To examine influences on other genes containing sequences with a small number of mismatches to siRNA, the same procedure as used in Example 5 was repeated to design siRNA against firefly luciferase, and the resulting siRNA was examined for its RNAi effect on the similar sequences with a small number of mismatches.

### [Designed siRNA Sequence (Prescribed Sequence Portion, Including Overhanging Portions of 2 Bases)]

3-36 gccattctatccgctggaagatg (SEQ ID NO: 817082)

### [Sequences Similar to Designed siRNA (Bases Indicated in Uppercase Letters Represent Mismatch Sites)]

3-36.R1

(SEQ ID NO: 817083)

gccattctatccgcGggCGgatg
3-36.R2

(SEQ ID NO: 817084)

gccattctatccgcCggGGgatg
3-36.R3

(SEQ ID NO: 817085)

gccattctatccgcGggaCgatg
3-36.R4

(SEQ ID NO: 817086)

gccattctatccgctggCGgatg
3-36.R5

(SEQ ID NO: 817087)

gccattctatccgctggaGgatg
3-36.R6

(SEQ ID NO: 817088)

gccattctatccgctgTaaTatg
3-36.R7

(SEQ ID NO: 817089)

gccattctatccgctAAaagatg
3-36.R8

(SEQ ID NO: 817090)

gccattctatccgctATaaAatg
3-36.L1

(SEQ ID NO: 817091)

gccGGCcCGtccgctggaagatg
3-36.L2

(SEQ ID NO: 817092)

gccCGtcCGtccgctggaagatg
3-36.L3

(SEQ ID NO: 817093)

gccGtCctGtccgctggaagatg
3-36.L4

(SEQ ID NO: 817094)

gccaCCcGatccgctggaagatg
3-36.L5

(SEQ ID NO: 817095)

gccattAtatccgctggaagatg
3-36.01A

(SEQ ID NO: 817096)

gcAattctatccgctggaagatg
3-36.01G

(SEQ ID NO: 817097)

gcGattctatccgctggaagatg
3-36.01U

(SEQ ID NO: 817098)

gcTattctatccgctggaagatg
3-36.19G

(SEQ ID NO: 817099)

gccattctatccgctggaagGtg
3-36.19C

(SEQ ID NO: 817100)

gccattctatccgctggaagCtg
3-36.19U

(SEQ ID NO: 817101)

gccattctatccgctggaagTtg

As a result of the assay shown in FIG. 36, in the case of designing base sequences of 19 bases, when genes other than the target gene contain similar sequences with mismatches of 2 or less bases, these similar sequence portions were confirmed to have a high probability of being targeted by RNA interference.

### Example 8

In this example, the siRNA sequences used were composed of 21-base sense strand RNA having the base sequences shown in Tables 1A to 1K (whose base sequences are shown in the column “siRNA-sense” of Table 1) and 21-base antisense strand RNA having the base sequences shown in Tables 1A to 1K (whose base sequences are shown in the column “siRNA-antisense” of Table 1, provided that the base sequences are shown in the direction from 3′ to 5′). As shown in Table 1, each siRNA was appropriately designed on the basis of each target sequence (see the column “Target Sequence”) located at a given position (see the column “Target Position”) in the coding region of each gene to be targeted by RNAi (see the column “Gene Name”; hereinafter also referred to as a target gene), particularly on the basis of the so-called prescribed sequence corresponding to a portion covering the third base from the 5′ end to the third base from the 3′ end of each target sequence. Each siRNA was then examined for its RNAi effect using human-derived HeLa cells. More specifically, even-numbered base sequences among SEQ ID NOs: 817102 to 817650 were examined as sense strands (siRNA-sense), while odd-numbered base sequences among SEQ ID NOs: 817102 to 817650 were examined as antisense strands (siRNA-antisense). Detailed procedures used in this example will be explained below.

### 1. Synthesis of siRNA

Double-stranded siRNA composed of sense and antisense strands was suitably designed according to the above rules of the present invention (the rules (a) to (d) described in [1], etc.) on the basis of the above prescribed sequence of each target gene. Based upon such design, the synthesis was entrusted to Proligo Japan for preparation. As to detailed synthetic procedures used here, sense and antisense strands having given base sequences as shown in the table were heated in a reaction liquid of 10 mM Tris-HCl (pH 7.5) and 20 mM NaCl at 90° C. for 3 minutes. Both strands were further incubated at 37° C. for 1 hour and then associated by standing until room temperature to form double-stranded siRNA. The double-stranded siRNA thus formed was subjected to electrophoresis using a 2% agarose gel in TBE buffer so as to confirm the association between sense and antisense strands.

### 2. Cell Cultivation

In this example, human-derived HeLa cells were used. The medium used for culturing HeLa cells (hereinafter also referred to as cell medium) was Dulbecco's Modified Eagle's medium (DMEM; manufactured by Invitrogen Corp.) which was supplemented with inactivated 10% fetal bovine serum (FBS; manufactured by Biomedicals, inc). In this medium, HeLa cells were cultured at 37° C. in the presence of 5% CO₂.

### 3. Target Gene to be Targeted by RNAi

Since HeLa cells which are uterine cervical cancer cells are used in this example, the individual genes shown in Table 1 which are endogenous genes in the HeLa cells and are highly expressed in these cells are targets for RNAi by siRNA, i.e., target genes for RNAi. In this example, HeLa cells were used to examine the RNAi effect of each siRNA on these genes, thereby studying the effect of siRNA on diseases and/or biological functions related to these genes (more specifically, see the columns “Related Disease”, “Biological Function Category” and/or “Reported Biological Function” of FIG. 46), i.e., a prophylactic/therapeutic effect on the diseases and/or a controlling effect on the biological functions. As an internal control gene, the endogenous GAPDH gene in HeLa cells was used in this study.

### 4. Introduction (Transfection) of siRNA into Cells

HeLa cells were first seeded at a density of 5×10⁴ cells/well into a 24-well plate and cultured for 24 hours under the cell culture conditions described above, followed by introducing 5 nM/well of siRNA. After the introduction, the HeLa cells were cultured at 37° C. for 24 hours. In this introduction process, Lipofectamine 2000 (manufactured by Invitrogen Corp.) was used as an introducing reagent, while DMEM was used as a medium for introduction. As to detailed procedures for introduction, Opti-MEM medium (manufactured by Invitrogen Corp.) containing Lipofectamine 2000 and siRNA was added to the cell medium, followed by culturing the HeLa cells to introduce siRNA into the cells. The HeLa cells thus introduced with siRNA are hereinafter referred to as an “evaluation sample.”

On the other hand, for correction of the level of target gene-derived mRNA in PCR described later, the following calibrator sample was prepared. The calibrator sample was prepared by the same treatment as used for the evaluation sample introduced with siRNA, except that Opti-MEM medium containing Lipofectamine 2000 but free from siRNA was added to the above cell medium to culture HeLa cells.

### 5. Measurement of RNAi Effect

After the above introduction was performed, HeLa cells were recovered for both evaluation and calibrator samples described above. The recovered cells were then provided for an ABI PRISM® 6700 Automated Nucleic Acid Workstation (manufactured by Applied Biosystems Corp.), and this apparatus was operated according to the manual to perform RNA extraction and cDNA synthesis by reverse transcription.

Subsequently, the resulting cDNA was used as a template to perform quantitative PCR in a 50-μl reaction system using SYBR Green PCR Master Mix (manufactured by Applied Biosystems Corp.). In this quantitative PCR, an ABI PRISM® 7900HT Sequence Detection System was used as a real-time monitoring apparatus and operated according to the manual. In addition, the PCR primers used were optimal primers obtained as a result of various studies.

In this example, the results obtained from PCR quantification were analyzed by a method called the “comparative Ct method.” With respect to this method, a detailed explanation is omitted here because an explanation of this method is disclosed in the home page of Applied Biosystems Corp. (http://www.appliedbiosystems.co.jp). The outline of this method is as follows: this method allows relative quantification by focusing on what number of cycles an evaluation sample reaches faster (or later) the Threshold Line, as compared to the calibrator sample.

More specifically, both evaluation and calibrator samples were first quantified by PCR to determine Ct1 that corresponds to a relative mRNA level including a target gene-derived base sequence(s) and Ct2 that corresponds to a relative mRNA level including an internal control gene-derived base sequence(s). In the following descriptions, the above Ct1 and Ct2 of an evaluation sample are referred to as “Ct1(E)” and “Ct2(E),” respectively. Likewise, the above Ct1 and Ct2 of the calibrator sample are referred to as “Ct1(C)” and “Ct2(C),” respectively.

As used herein, “Ct” denotes the number of cycles required before reaching the Threshold Line, and more specifically is defined by the following Equation (1). It should be noted that the amplification efficiency is set to 1 in this case. With respect to the numeric characters following Ct, “1” means a mRNA level derived from a target gene and “2” means a mRNA level derived from the internal control gene. With respect to the designations (E) and (C) following Ct, “E” means an evaluation sample and “C” means the calibration sample. Regardless of the designations “1”, “2”, “E” and “C”, “Ct” is defined as follows:

Ct=(log [DNA]t−log [DNA]0)/log 2 (1)

wherein [DNA]t represents the amount of DNA at the time of reaching the Threshold Line, and [DNA]0 represents the initial amount of cDNA reverse-transcribed from mRNA.

Ct1(E), Ct2(E), Ct1(C) and Ct2(C) thus obtained by PCR quantification were subjected to and analyzed by the comparative Ct method to obtain a RQ value used for evaluating the RNAi effect of siRNA. The RQ value is a relative mRNA level of a target gene in an evaluation sample when the mRNA level of the target gene in the calibration sample is set to 1. More specifically, the RQ value is defined by the following Equation (2):

RQ=2^{(-ΔΔCt)} (2)

wherein ΔΔCt is defined by the following Equation (3):

ΔΔCt=ΔCt(E)−ΔCt(C) (3)

wherein Δ [symbol] Ct(E) is defined by the following Equation (4) and ΔCt(C) is defined by the following Equation (5):

ΔCt(E)=Ct1(E)−Ct2(E) (4)

ΔCt(C)=Ct1(C)−Ct2(C) (5).

### It should be noted that the designations “1”, “2”, “E” and “C” in Equations (2) to (5) are as defined above.

### 6. Evaluation of RNAi Effect

The RQ values thus obtained are shown in Tables 1A to 1K. In Table 1, the data in the columns “Gene Name” and “refseq_NO.”, portions actually targeted by RNAi within the sequences listed in the column “Target Sequence” and the definition of “Target Position” are as described above for FIG. 46 in the section “BRIEF DESCRIPTION OF THE DRAWINGS” of this specification.

In this example, on the basis of the RQ values thus calculated (see the column “RQ value” of Table 1), each siRNA was evaluated for RNAi effect on its target gene. As is evident from the table, siRNA sequences composed of sense strands having even-numbered base sequences among SEQ ID NOs: 817102 to 817650 and antisense strands having odd-numbered base sequences among SEQ ID NOs: 817102 to 817650 were all found to have a RQ value less than 1 and almost all found to have a RQ value less than 0.5, thus indicating that these siRNA sequences caused a 50% or more inhibition of the expression of the target genes shown in Table 1. Such an RNAi effect of each siRNA was also achieved when repeating the same procedure as shown above with COS cells.

Moreover, in light of the results from Example 8 showing that all the 294 tested siRNA sequences falling within the present invention were found to produce an RNAi effect, it was indicated that the polynucleotides (siRNA) of the present invention effectively produced an RNAi effect against their target genes in mammalian cells and caused a 50% or more inhibition of gene expression.

In Examples 1 to 8, the cases using siRNA sequences whose sense and antisense strands are each composed of RNA were shown. The same results as in Examples 1 to 8 are also obtained in the case of using siRNA having a chimeric structure. Although the detailed explanation for the case of siRNA having a chimeric structure is omitted here, for example, when siRNA having a chimeric structure is used in Example 8, this siRNA structurally differs in the following point from the siRNA sequences of Example 8 which are composed of sense and antisense strands shown under SEQ ID NOs: 817102 to 817651.

Namely, siRNA sequences of chimeric structure have the same base sequences as siRNA sequences composed of sense and antisense strands shown in Table 1 under SEQ ID NOs: 817102 to 817651. However, a portion of 8 to 12 nucleotides (e.g., 10 nucleotides, preferably 11 nucleotides, more preferably 12 nucleotides) from the 3′ end of the sense strand (for example, “A” in the case of the sense strand shown in Table 1 under SEQ ID NO: 8102) and a portion of 8 to 12 nucleotides (e.g., 10 nucleotides, preferably 11 nucleotides, more preferably 12 nucleotides) from the 5′ end of the antisense strand (for example, “A” in the case of the antisense strand shown in Table 1 under SEQ ID NO: 8103) are both composed of DNA. Thus, siRNA sequences of chimeric structure differ from the siRNA sequences shown under SEQ ID NOs: 817102 to 817651 in that U in the above polynucleotide portions is replaced by T within the base sequences of the sense and antisense strands shown in Table 1.

TABLE 1

| | | | | | SEQ | | SEQ | | SEQ |
|---|---|---|---|---|---|---|---|---|---|
| Gane | | | Target | | ID | | ID | | ID |
| Name | refseq_ID | RQ | Position | Target sequence | NO | siRNA-sense | NO | siRNA-antisense | NO |
| PSEN1 | NM_000021.2 | 0.23 | 532 | tggtcgtggctaccattaagtca | 307985 | GUCGUGGCUACCAUUAAGUCA | 817102 | ACUUAAUGGUAGCCACGACCA | 817103 |
| JAG1 | NM_000214.1 | 0.28 | 794 | ctggccgaggtcctatacgttgc | 147021 | GGCCGAGGUCCUAUACGUUGC | 817104 | AACGUAUAGGACCUCGGCCAG | 817105 |
| POLR2A | NM_000937.2 | 0.34 | 2425 | tcctcatcgagggtcatactatt | 36223 | CUCAUCGAGGGUCAUACUAUU | 817106 | UAGUAUGACCCUCGAUGAGGA | 817107 |
| CDC6 | NM_001254.3 | 0.35 | 383 | gtctgggcgatgacaacctatgc | 76037 | CUGGGCGAUGACAACCUAUGC | 817108 | AUAGGUUGUCAUCGCCCAGAC | 817109 |
| CSE1L | NM_001316.2 | 0.15 | 393 | gccgatcgagtggccattaaagc | 128329 | CGAUCGAGUGGCCAUUAAAGC | 817110 | UUUAAUGGCCACUCGAUCGGC | 817111 |
| HDAC2 | NM_001527.1 | 0.15 | 1110 | tggctacacaatccgtaatgttg | 4714 | GCUACACAAUCCGUAAUGUUG | 817112 | ACAUUACGGAUUGUGUAGCCA | 817113 |
| HIF1A | NM_001530.2 | 0.2 | 809 | aactagccgaggaagaactatga | 237 | CUAGCCGAGGAAGAACUAUGA | 817114 | AUAGUUCUUCCUCGGCUAGUU | 817115 |
| IGFBP4 | NM_001552.1 | 0.064 | 706 | aagcacttcgccaaaattcgaga | 124916 | GCACUUCGCCAAAAUUCGAGA | 817116 | UCGAAUUUUGGCGAAGUGCUU | 817117 |
| CDC2 | NM_001786.2 | 0.18 | 656 | tggggtcagctcgttactcaact | 75723 | GGGUCAGCUCGUUACUCAACU | 817118 | UUGAGUAACGAGCUGACCCCA | 817119 |
| CDK2 | NM_001798.2 | 0.21 | 689 | tggagtccctgttcgtacttaca | 76134 | GAGUCCCUGUUCGUACUUACA | 817120 | UAAGUACGAACAGGGACUCCA | 817121 |
| CDK7 | NM_001799.2 | 0.3 | 575 | gggagccccaatagagcttatac | 76204 | GAGCCCCAAUAGAGCUUAUAC | 817122 | AUAAGCUCUAUUGGGGCUCCC | 817123 |
| CUTL1 | NM_001913.2 | 0.36 | 139 | gtccagaaagcggcttatcgaac | 2624 | CCAGAAAGCGGCUUAUCGAAC | 817124 | UCGAUAAGCCGCUUUCUGGAC | 817125 |
| E2F4 | NM_001950.3 | 0.2 | 1220 | cccgggagaccacgattatatct | 2910 | CGGGAGACCACGAUUAUAUCU | 817126 | AUAUAAUCGUGGUCUCCCGGG | 817127 |
| GNB1 | NM_002074.2 | 0.072 | 672 | tgcggtggcctggataacatttg | 192277 | CGGUGGCCUGGAUAACAUUUG | 817128 | AAUGUUAUCCAGGCCACCGCA | 817129 |
| HSPA4 | NM_002154.3 | 0.055 | 578 | aggtataaaggtgacatatatgg | 85169 | GUAUAAAGGUGACAUAUAUGG | 817130 | AUAUAUGUCACCUUUAUACCU | 817131 |
| KPNA1 | NM_002264.1 | 0.2 | 520 | ttctcttcagacccgaattgtga | 133261 | CUCUUCAGACCCGAAUUGUGA | 817132 | ACAAUUCGGGUCUGAAGAGAA | 817133 |
| KPNA3 | NM_002267.2 | 0.074 | 1921 | aggaggtacctacaattttgatc | 177084 | GAGGUACCUACAAUUUUGAUC | 817134 | UCAAAAUUGUAGGUACCUCCU | 817135 |
| KPNA4 | NM_002268.3 | 0.094 | 1595 | tagtactcgatggactaagtaat | 269352 | GUACUCGAUGGACUAAGUAAU | 817136 | UACUUAGUCCAUCGAGUACUA | 817137 |
| PAWR | NM_002583.2 | 0.21 | 984 | gtgggttccctagatataacagg | 113162 | GGGUUCCCUAGAUAUAACAGG | 817138 | UGUUAUAUCUAGGGAACCCAC | 817139 |
| POLD1 | NM_002691.1 | 0.29 | 2216 | ggggttcggacgtcagatgatcg | 35766 | GGUUCGGACGUCAGAUGAUCG | 817140 | AUCAUCUGACGUCCGAACCCC | 817141 |
| POLR2G | NM_002696.1 | 0.11 | 586 | tggctccctgatggacgattact | 53520 | GCUCCCUGAUGGACGAUUACU | 817142 | UAAUCGUCCAUCAGGGAGCCA | 817143 |
| PRKACB | NM_002731.1 | 0.1 | 944 | cacgacagattggattgctattt | 96985 | CGACAGAUUGGAUUGCUAUUU | 817144 | AUAGCAAUCCAAUCUGUCGUG | 817145 |
| PRKCA | NM_002737.2 | 0.19 | 429 | ctgcgatatgaacgttcacaagc | 97011 | GCGAUAUGAACGUUCACAAGC | 817146 | UUGUGAACGUUCAUAUCGCAG | 817147 |
| MAPK1 | NM_002745.2 | 0.086 | 383 | aagttcgagtagctatcaagaaa | 89815 | GUUCGAGUAGCUAUCAAGAAA | 817148 | UCUUGAUAGCUACUCGAACUU | 817149 |
| MAPK9 | NM_002752.3 | 0.22 | 261 | atcgtgaacttgtcctcttaaaa | 90069 | CGUGAACUUGUCCUCUUAAAA | 817150 | UUAAGAGGACAAGUUCACGAU | 817151 |
| MAP2K1 | NM_002755.2 | 0.21 | 114 | ccccgacggctctgcagttaacg | 88938 | CCGACGGCUCUGCAGUUAACG | 817152 | UUAACUGCAGAGCCGUCGGGG | 817153 |
| PSMA2 | NM_002787.3 | 0.022 | 56 | ttcagcccgtctggtaaacttgt | 185145 | CAGCCCGUCUGGUAAACUUGU | 817154 | AAGUUUACCAGACGGGCUGAA | 817155 |
| PSMA3 | NM_002788.2 | 0.074 | 599 | atgacctgccgtgatatcgttaa | 185178 | GACCUGCCGUGAUAUCGUUAA | 817156 | AACGAUAUCACGGCAGGUCAU | 817157 |
| PSMA4 | NM_002789.3 | 0.1 | 183 | gtcgcttataccaagttgaatat | 185191 | CGCUUAUACCAAGUUGAAUAU | 817158 | AUUCAACUUGGUAUAAGCGAC | 817159 |
| PSMA5 | NM_002790.2 | 0.39 | 107 | tacgacaggggcgtgaatacttt | 185211 | CGACAGGGGCGUGAAUACUUU | 817160 | AGUAUUCACGCCCCUGUCGUA | 817161 |
| PSMA6 | NM_002791.1 | 0.07 | 129 | ccggttttgaccgccacattacc | 53630 | GGUUUUGACCGCCACAUUACC | 817162 | UAAUGUGGCGGUCAAAACCGG | 817163 |
| PSMA7 | NM_002792.2 | 0.13 | 346 | cgccgatgcaaggatagtcatca | 185235 | CCGAUGCAAGGAUAGUCAUCA | 817164 | AUGACUAUCCUUGCAUCGGCG | 817165 |
| PSMB1 | NM_002793.2 | 0.035 | 130 | tgcgattttcgccctacgttttc | 185241 | CGAUUUUCGCCCUACGUUUUC | 817166 | AAACGUAGGGCGAAAAUCGCA | 817167 |
| PSMB2 | NM_002794.3 | 0.66 | 530 | cagtatcctcgaccgatactaca | 185279 | GUAUCCUCGACCGAUACUACA | 817168 | UAGUAUCGGUCGAGGAUACUG | 817169 |
| PSMB3 | NM_002795.2 | 0.063 | 312 | aggtcggcagatcaaaccttata | 185290 | GUCGGCAGAUCAAACCUUAUA | 817170 | UAAGGUUUGAUCUGCCGACCU | 817171 |
| PSMB4 | NM_002796.2 | 0.052 | 317 | ctctggcgactacgctgatttcc | 185302 | CUGGCGACUACGCUGAUUUCC | 817172 | AAAUCAGCGUAGUCGCCAGAG | 817173 |
| PSMB6 | NM_002798.1 | 0.067 | 683 | gggtagagcggcaagtacttttg | 185333 | GUAGAGCGGCAAGUACUUUUG | 817174 | AAGUACUUGCCGCUCUACCC | 817175 |
| PSMC3 | NM_002804.3 | 0.089 | 1197 | ccgccttgaccgcaagatagagt | 98373 | GCCUUGACCGCAAGAUAGAGU | 817176 | UCUAUCUUGCGGUCAAGGCGG | 817177 |
| PSMD7 | NM_002811.3 | 0.11 | 477 | ctgtcctaattccgtattggtca | 57203 | GUCCUAAUUCCGUAUUGGUCA | 817178 | ACCAAUACGGAAUUAGGACAG | 817179 |
| RAF1 | NM_002880.2 | 0.41 | 897 | gtcgacatccacacctaatgtcc | 98850 | CGACAUCCACACCUAAUGUCC | 817180 | ACAUUAGGUGUGGAUGUCGAC | 817181 |
| SHC1 | NM_003029.3 | 0.12 | 601 | gccgagtatgtcgcctatgttgc | 253033 | CGAGUAUGUCGCCUAUGUUGC | 817182 | AACAUAGGCGACAUACUCGGC | 817183 |
| SP3 | NM_003111.1 | 0.4 | 2324 | agctgcgcgagatgatactttga | 40777 | CUGCGCGAGAUGAUACUUUGA | 817184 | AAAGUAUCAUCUCGCGCAGCU | 817185 |
| TCF7 | NM_003202.1 | 0.23 | 92 | accgtctactccgccttcaatct | 41852 | CGUCUACUCCGCCUUCAAUCU | 817186 | AUUGAAGGCGGAGUAGACGGU | 817187 |
| TEAD4 | NM_003213.1 | 0.16 | 1386 | tgctgtgcattgcctatgtcttt | 13093 | CUGUGCAUUGCCUAUGUCUUU | 817188 | AGACAUAGGCAAUGCACAGCA | 817189 |
| TMPO | NM_003276.1 | 0.14 | 1609 | ctcactaccttaggtctagaagt | 127873 | CACUACCUUAGGUCUAGAAGU | 817190 | UUCUAGACCUAAGGUAGUGAG | 817191 |
| YWHAB | NM_003404.3 | 0.072 | 769 | cagcctacacacccaattcgtct | 126595 | GCCUACACACCCAAUUCGUCU | 817192 | ACGAAUUGGGUGUGUAGGCUG | 817193 |
| YWHAH | NM_003405.2 | 0.094 | 824 | cacactaaacgaggattcctata | 126608 | CACUAAACGAGGAUUCCUAUA | 817194 | UAGGAAUCCUCGUUUAGUGUG | 817195 |
| OGT | NM_003605.3 | 0.16 | 449 | ctggcagaagcttattcgaattt | 134296 | GGCAGAAGCUUAUUCGAAUUU | 817196 | AUUCGAAUAAGCUUCUGCCAG | 817197 |
| PPP2CB | NM_004156.1 | 0.23 | 801 | cagtgcacccaattactgttatc | 162283 | GUGCACCCAAUUACUGUUAUC | 817198 | UAACAGUAAUUGGGUGCACUG | 817199 |
| SCYE1 | NM_004757.2 | 0.11 | 305 | ctgcacgctaattctatggtttc | 49202 | GCACGCUAAUUCUAUGGUUUC | 817200 | AACCAUAGAAUUAGCGUGCAG | 817201 |
| HDAC1 | NM_004964.2 | 0.15 | 870 | tcggttaggttgcttcaatctaa | 4672 | GGUUAGGUUGCUUCAAUCUAA | 817202 | AGAUUGAAGCAACCUAACCGA | 817203 |
| PSMD5 | NM_005047.2 | 0.15 | 1476 | gtgaagggccatactatgtgaaa | 323491 | GAAGGGCCAUACUAUGUGAAA | 817204 | UCACAUAGUAUGGCCCUUCAC | 817205 |
| CEBPB | NM_005194.2 | 0.31 | 1001 | agcacagcgacgagtacaagatc | 2153 | CACAGCGACGAGUACAAGAUC | 811206 | UCUUGUACUCGUCGCUGUGCU | 817207 |
| EGFR | NM_005228.2 | 0.27 | 2387 | cccgtcgctatcaaggaattaag | 81171 | CGUCGCUAUCAAGGAAUUAAG | 817208 | UAAUUCCUUGAUAGCGACGGG | 817209 |
| ELK1 | NM_005229.2 | 0.22 | 419 | ggccttgcggtactactatgaca | 3142 | CCUUGCGGUACUACUAUGACA | 817210 | UCAUAGUAGUACCGCAAGGCC | 817211 |
| EWSR1 | NM_005243.1 | 0.22 | 631 | ctctacacagccgactagttatg | 51496 | CUACACAGCCGACUAGUUAUG | 817212 | UAACUAGUCGGCUGUGUAGAG | 817213 |
| HCFC1 | NM_005334.1 | 0.22 | 5339 | gggcaccgtccctgactataacc | 4624 | GCACCGUCCCUGACUAUAACC | 817214 | UUAUAGUCAGGGACGGUGCCC | 817215 |
| JUND | NM_005354.2 | 0.22 | 1053 | ctcgcgcctggaagagaaagtga | 5612 | CGCGCCUGGAAGAGAAAGUGA | 817216 | ACUUUCUCUUCCAGGCGCGAG | 817217 |
| YES1 | NM_005433.3 | 0.1 | 839 | ttgcgactagaggttaaactagg | 107693 | GCGACUAGAGGUUAAACUAGG | 817218 | UAGUUUAACCUCUAGUCGCAA | 817219 |
| TAF6 | NM_005641.2 | 0.13 | 748 | ttgactacgccttgaagctaaag | 41513 | GACUACGCCUUGAAGCUAAAG | 817220 | UUAGCUUCAAGGCGUAGUCAA | 817221 |
| TAF7 | NM_005642.2 | 0.39 | 1133 | ctggaaccacggaattactctgc | 112459 | GGAACCACGGAAUUACUCUGC | 817222 | AGAGUAAUUCCGUGGUUCCAG | 817223 |
| PRKCN | NM_005813.2 | 0.25 | 3090 | aactcgcattggagaacgttaca | 97488 | CUCGCAUUGGAGAACGUUACA | 817224 | UAACGUUCUCCAAUGCGAGUU | 817225 |
| PA2G4 | NM_006191.1 | 0.08 | 752 | gaggtacatgaagtatatgctgt | 186582 | GGUACAUGAAGUAUAUGCUGU | 817226 | AGCAUAUACUUCAUGUACCUC | 817227 |
| TAF10 | NM_006284.2 | 0.13 | 461 | gcctcagacccacgcataattcg | 12455 | CUCAGACCCACGCAUAAUUCG | 817228 | AAUUAUGCGUGGGUCUGAGGC | 817229 |
| COPS5 | NM_006837.2 | 0.22 | 726 | atgcaatcgggtggtatcatagc | 56199 | GCAAUCGGGUGGUAUCAUAGC | 817230 | UAUGAUACCACCCGAUUGCAU | 817231 |
| STAT1 | NM_007315.2 | 0.21 | 2177 | aaggggccatcacattcacatgg | 12048 | GGGGCCAUCACAUUCACAUGG | 817232 | AUGUGAAUGUGAUGGCCCCUU | 817233 |
| GALNT1 | NM_020474.2 | 0.092 | 1203 | tagattatggagatatatcgtca | 161846 | GAUUAUGGAGAUAUAUCGUCA | 817234 | ACGAUAUAUCUCCAUAAUCUA | 817235 |
| CDKN2A | NM_000077.3 | 0.16 | 677 | ggcaccagaggcagtaaccatgc | 219272 | CACCAGAGGCAGUAACCAUGC | 817236 | AUGGUUACUGCCUCUGGUGCC | 817237 |
| RB1 | NM_000321.1 | 0.094 | 2701 | agcgaccgtgtgctcaaaagaag | 10143 | CGACCGUGUGCUCAAAAGAAG | 817238 | UCUUUUGAGCACACGGUCGCU | 817239 |
| CD44 | NM_000610.2 | 0.16 | 233 | ctggcgcagatcgatttgaatat | 126722 | GGCGCAGAUCGAUUUGAAUAU | 817240 | AUUCAAAUCGAUCUGCGCCAG | 817241 |
| COMT | NM_000754.2 | 0.093 | 922 | gtgcacacactaccaatcgttcc | 165318 | GCACACACUACCAAUCGUUCC | 817242 | AACGAUUGGUAGUGUGUGCAC | 817243 |
| GSTP1 | NM_000852.2 | 0.14 | 624 | tacgtgaacctccccatcaatgg | 216683 | CGUGAACCUCCCCAUCAAUGG | 817244 | AUUGAUGGGGAGGUUCACGUA | 817245 |
| IGF1R | NM_000875.2 | 0.27 | 279 | cacggtcattaccgagtacttgc | 85645 | CGGUCAUUACCGAGUACUUGC | 817246 | AAGUACUCGGUAAUGACCGUG | 817247 |
| ARHA | NM_001664.2 | 0.098 | 371 | tacccagataccgatgttatact | 108327 | CCCAGAUACCGAUGUUAUACU | 817248 | UAUAACAUCGGUAUCUGGGUA | 817249 |
| CTSC | NM_001814.2 | 0.29 | 236 | cggttcccagcgcgatgtcaact | 188060 | GUUCCCAGCGCGAUGUCAACU | 817250 | UUGACAUCGCGCUGGGAACCG | 817251 |
| FN1 | NM_002026.1 | 0.13 | 473 | acctaggcaatgcgttggtttgt | 126771 | CUAGGCAAUGCGUUGGUUUGU | 817252 | AAACCAACGCAUUGCCUAGGU | 817253 |
| LGALS1 | NM_002305.2 | 0.047 | 367 | agctgccagatggatacgaattc | 174842 | CUGCCAGAUGGAUACGAAUUC | 817254 | AUUCGUAUCCAUCUGGCAGCU | 817255 |
| NRAS | NM_002524.2 | 0.11 | 445 | cagtgccatgagagaccaataca | 109675 | GUGCCAUGAGAGACCAAUACA | 817256 | UAUUGGUCUCUCAUGGCACUG | 817257 |
| PCNA | NM_002592.2 | 0.087 | 526 | cggataccttggcgctagtattt | 34625 | GAUACCUUGGCGCUAGUAUUU | 817258 | AUACUAGCGCCAAGGUAUCCG | 817259 |
| PKM2 | NM_002654.3 | 0.08 | 565 | tgctgtggctctagacactaaag | 166519 | CUGUGGCUCUAGACACUAAAG | 817260 | UUAGUGUCUAGAGCCACAGCA | 817261 |
| RXRA | NM_002957.3 | 0.47 | 1342 | tgcgctccatcgggctcaaatgc | 10866 | CGCUCCAUCGGGCUCAAAUGC | 817262 | AUUUGAGCCCGAUGGAGCGCA | 817263 |
| S100A4 | NM_002961.2 | 0.12 | 152 | agctcaacaagtcagaactaaag | 152374 | CUCAACAAGUCAGAACUAAAG | 817264 | UUAGUUCUGACUUGUUGAGCU | 817265 |
| TFAP2A | NM_003220.1 | 0.37 | 978 | tacgtgtgcgaaaccgaatttcc | 546 | CGUGUGCGAAACCGAAUUUCC | 817266 | AAAUUCGGUUUCGCACACGUA | 817267 |
| EIF3S10 | NM_003750.1 | 0.28 | 145 | ccctcaaacgcgccaacgaattt | 56509 | CUCAAACGCGCCAACGAAUUU | 817268 | AUUCGUUGGCGCGUUUGAGGG | 817269 |
| EIF3S9 | NM_003751.2 | 0.12 | 641 | gggacccgaccgacttgagaaac | 51229 | GACCCGACCGACUUGAGAAAC | 817270 | UUCUCAAGUCGGUCGGGUCCC | 817271 |
| EIF3S8 | NM_003752.2 | 0.1 | 417 | ctgacctagaggactatcttaat | 56770 | GACCUAGAGGACUAUCUUAAU | 817272 | UAAGAUAGUCCUCUAGGUCAG | 817273 |
| EIF3S7 | NM_003753.2 | 0.15 | 1729 | ctcggtaccacgtgaaagactcc | 56765 | CGGUACCACGUGAAAGACUCC | 817274 | AGUCUUUCACGUGGUACCGAG | 817275 |
| EIF3S4 | NM_003755.2 | 0.12 | 182 | aggtcatcaacggaaacataaag | 51220 | GUCAUCAACGGAAACAUAAAG | 817276 | UUAUGUUUCCGUUGAUGACCU | 817277 |
| EIF3S3 | NM_003756.1 | 0.19 | 601 | aagaagtgccgattgtaattaaa | 56655 | GAAGUGCCGAUUGUAAUUAAA | 817278 | UAAUUACAAUCGGCACUUCUU | 817279 |
| EIF3S2 | NM_003757.1 | 0.11 | 46 | agcggtccattacgcagattaag | 56617 | CGGUCCAUUACGCAGAUUAAG | 817280 | UAAUCUGCGUAAUGGACCGCU | 817281 |
| EIF3S1 | NM_003758.1 | 0.16 | 442 | gacctcgaattagcaaaggaaac | 56505 | CCUCGAAUUAGCAAAGGAAAC | 817282 | UUCCUUUGCUAAUUCGAGGUC | 817283 |
| BAG1 | NM_004323.2 | 0.25 | 697 | atggttgccgggtcatgttaatt | 129118 | GGUUGCCGGGUCAUGUUAAUU | 817284 | UUAACAUGACCCGGCAACCAU | 817285 |
| AKT1 | NM_005163.1 | 0.21 | 239 | aacgaggggagtacatcaagacc | 71961 | CGAGGGGAGUACAUCAAGACC | 817286 | UCUUGAUGUACUCCCCUCGUU | 817287 |
| NORG1 | NM_006096.2 | 0.074 | 567 | gcctacatcctaactcgatttgc | 236862 | CUACAUCCUAACUCGAUUUGC | 817288 | AAAUCGAGUUAGGAUGUAGGC | 817289 |
| TSG101 | NM_006292.2 | 0.13 | 943 | atggttacccgtttagatcaaga | 43049 | GGUUACCCGUUUAGAUCAAGA | 817290 | UUGAUCUAAACGGGUAACCAU | 817291 |
| BRCA1 | NM_007294.1 | 0.22 | 4329 | gagggataccatgcaacataacc | 16042 | GGGAUACCAUGCAACAUAACC | 817292 | UUAUGUUGCAUGGUAUCCCUC | 817293 |
| NOTCH2 | NM_024408.2 | 0.085 | 6047 | cgcaaccgagtaactgatctaga | 149219 | CAACCGAGUAACUGAUCUAGA | 817294 | UAGAUCAGUUACUCGGUUGCG | 817295 |
| ARHC | NM_175744.3 | 0.11 | 194 | gtctacgtccctactgtctttga | 108338 | CUACGUCCCUACUGUCUUUGA | 817296 | AAAGACAGUAGGGACGUAGAC | 817297 |
| BLM | NM_000057.1 | 0.35 | 1998 | gagcgtttccaaagtcttagttt | 22786 | GCGUUUCCAAAGUCUUAGUUU | 817298 | ACUAAGACUUUGGAAACGCUC | 817299 |
| GSN | NM_000177.3 | 0.13 | 740 | cagcaatcggtatgaaagactga | 113910 | GCAAUCGGUAUGAAAGACUGA | 817300 | AGUCUUUCAUACCGAUUGCUG | 817301 |
| MLH1 | NM_000249.2 | 0.19 | 847 | aaccatcgtctggtagaatcaac | 91691 | CCAUCGUCUGGUAGAAUCAAC | 817302 | UGAUUCUACCAGACGAUGGUU | 817303 |
| MSH2 | NM_000251.1 | 0.14 | 1282 | accgactctatcagggtataaat | 16366 | CGACUCUAUCAGGGUAUAAAU | 817304 | UUAUACCCUGAUAGAGUCGGU | 817305 |
| SOD1 | NM_000454.4 | 0.037 | 343 | tggtgtggccgatgtgtctattg | 167035 | GUGUGGCCGAUGUGUCUAUUG | 817306 | AUAGACACAUCGGCCACACCA | 817307 |
| TOP2A | NM_001067.2 | 0.24 | 2525 | ctgctagtccacgatacatcttt | 42581 | GCUAGUCCACGAUACAUCUUU | 817308 | AGAUGUAUCGUGGACUAGCAG | 817309 |
| TOP2B | NM_001068.2 | 0.11 | 1011 | aggtggacggcacgtggattatg | 42675 | GUGGACGGCACGUGGAUUAUG | 817310 | UAAUCCACGUGCCGUCCACCU | 817311 |
| TUBG1 | NM_001070.3 | 0.11 | 603 | gtggtggtccagccttacaattc | 111063 | GGUGGUCCAGCCUUACAAUUC | 817312 | AUUGUAAGGCUGGACCACCAC | 817313 |
| SLC25A5 | NM_001152.1 | 0.035 | 670 | tgcttccggatcccaagaacact | 181277 | CUUCCGGAUCCCAAGAACACU | 817314 | UGUUCUUGGGAUCCGGAAGCA | 817315 |
| ANXA11 | NM_001157.2 | 0.17 | 1685 | cacgacatctcgggagatacttc | 128933 | CGACAUCUCGGGAGAUACUUC | 817316 | AGUAUCUCCCGAGAUGUCGUG | 817317 |
| AP2B1 | NM_001282.1 | 0.19 | 714 | tgccgtagcggcattatctgaaa | 273115 | CCGUAGCGGCAUUAUCUGAAA | 817318 | UCAGAUAAUGCCGCUACGGCA | 817319 |
| GTF2I | NM_001518.2 | 0.37 | 978 | atgctgacaggtcaatactatct | 4585 | GCUGACAGGUCAAUACUAUCU | 817320 | AUAGUAUUGACCUGUCAGCAU | 817321 |
| IGFBP7 | NM_001553.1 | 0.045 | 488 | tgcgagcaaggtccttccatagt | 124925 | CGAGCAAGGUCCUUCCAUAGU | 817322 | UAUGGAAGGACCUUGCUCGCA | 817323 |
| AXL | NM_001699.3 | 0.14 | 1857 | aagaaggagacccgttatggaga | 73957 | GAAGGAGACCCGUUAUGGAGA | 817324 | UCCAUAACGGGUCUCCUUCUU | 817325 |
| CAPG | NM_001747.1 | 0.17 | 790 | ggccgcagctctgtataaggtct | 113927 | CCGCAGCUCUGUAUAAGGUCU | 817326 | ACCUUAUACAGAGCUGCGGCC | 817327 |
| DUT | NM_001948.2 | 0.16 | 419 | tgcgaacggattttttatccaga | 165338 | CGAACGGAUUUUUUAUCCAGA | 817328 | UGGAUAAAAAAUCCGUUCGCA | 817329 |
| JUP | NM_002230.1 | 0.18 | 1133 | atccgtgtgtcccagcaataagc | 120947 | CCGUGUGUCCCAGCAAUAAGC | 817330 | UUAUUGCUGGGACACACGGAU | 817331 |
| KPNB1 | NM_002265.4 | 0.043 | 2885 | ggcggagatcgaagactaacaaa | 157208 | CGGAGAUCGAAGACUAACAAA | 817332 | UGUUAGUCUUCGAUCUCCGCC | 817333 |
| MYH9 | NM_002473.3 | 0.12 | 465 | caccgcctacaggagtatgatgc | 92428 | CCGCCUACAGGAGUAUGAUGC | 817334 | AUCAUACUCCUGUAGGCGGUG | 817335 |
| PFN2 | NM_002628.2 | 0.08 | 82 | cggctactgcgacgccaaatacg | 118724 | GCUACUGCGACGCCAAAUACG | 817336 | UAUUUGGCGUCGCAGUAGCCG | 817337 |
| PPP1CA | NM_002708.2 | 0.081 | 239 | ctcaagatctgcggtgacataca | 162170 | CAAGAUCUGCGGUGACAUACA | 817338 | UAUGUCACCGCAGAUCUUGAG | 817339 |
| PPP1CB | NM_002709.1 | 0.15 | 1028 | ttgctaaacgacagttggtaacc | 162204 | GCUAAACGACAGUUGGUAACC | 817340 | UUACCAACUGUCGUUUAGCAA | 817341 |
| PPP1CC | NM_002710.1 | 0.32 | 1084 | aacgcctccaaggggtatgatca | 162234 | CGCCUCCAAGGGGUAUGAUCA | 817342 | AUCAUACCCCUUGGAGGCGUU | 817343 |
| THBS1 | NM_003246.2 | 0.28 | 3224 | caccgaaagggacgatgactatg | 153751 | CCGAAAGGGACGAUGACUAUG | 817344 | UAGUCAUCGUCCCUUUCGGUG | 817345 |
| TTC1 | NM_003314.1 | 0.16 | 879 | accggctcgtactccatcaattt | 136959 | CGGCUCGUACUCCAUCAAUUU | 817346 | AUUGAUGGAGUACGAGCCGGU | 817347 |
| TXNRD1 | NM_003330.2 | 0.15 | 1777 | gacgattccgtcaagagataaca | 167072 | CGAUUCCGUCAAGAGAUAACA | 817348 | UUAUCUCUUGACGGAAUCGUC | 817349 |
| VIL2 | NM_003379.3 | 0.078 | 458 | aggaatccttagcgatgagatct | 292759 | GAAUCCUUAGCGAUGAGAUCU | 817350 | AUCUCAUCGCUAAGGAUUCCU | 817351 |
| VIM | NM_003380.1 | 0.073 | 1447 | tcctgattaagacggttgaaact | 287581 | CUGAUUAAGACGGUUGAAACU | 817352 | UUUCAACCGUCUUAAUCAGGA | 817353 |
| EXO1 | NM_003686.3 | 0.25 | 1631 | tggaacgagtgattagtactaaa | 26320 | GAACGAGUGAUUAGUACUAAA | 817354 | UAGUACUAAUCACUCGUUCCA | 817355 |
| RUVBL1 | NM_003707.1 | 0.085 | 215 | gaggcatgtggcgtcatagtaga | 100083 | GGCAUGUGGCGUCAUAGUAGA | 817356 | UACUAUGACGCCACAUGCCUC | 817357 |
| ADAM9 | NM_003816.1 | 0.15 | 1051 | agccacgcaggcgggattaatgt | 155099 | CCACGCAGGCGGGAUUAAUGU | 817358 | AUUAAUCCCGCCUGCGUGGCU | 817359 |
| TNFRSF10B | NM_003842.3 | 0.41 | 945 | tgcagccgtagtcttgattgtgg | 127913 | CAGCCGUAGUCUUGAUUGUGG | 817360 | ACAAUCAAGACUACGGCUGCA | 817361 |
| SHMT1 | NM_004169.3 | 0.12 | 975 | gccgagctggcatgatcttctac | 214683 | CGAGCUGGCAUGAUCUUCUAC | 817362 | AGAAGAUCAUGCCAGCUCGGC | 817363 |
| CAD | NM_004341.2 | 0.36 | 5394 | ggggaggttgcctatatcgatgg | 74903 | GGAGGUUGCCUAUAUCGAUGG | 817364 | AUCGAUAUAGGCAACCUCCCC | 817365 |
| CSK | NM_004383.1 | 0.22 | 1132 | gacgcaactgcggcatagcaacc | 77629 | CGCAACUGCGGCAUAGCAACC | 817366 | UUGCUAUGCCGCAGUUGCGUC | 817367 |
| XPC | NM_004628.3 | 0.31 | 584 | ttcggagggcgatgaaacgtttc | 17754 | CGGAGGGCGAUGAAACGUUUC | 817368 | AACGUUUCAUCGCCCUCCGAA | 817369 |
| HGS | NM_004712.3 | 0.35 | 1883 | cccaatgcacggcgtgtacatga | 157053 | CAAUGCACGGCGUGUACAUGA | 817370 | AUGUACACGCCGUGCAUUGGG | 817371 |
| LRRFIP1 | NM_004735.2 | 0.093 | 1426 | gtgtcctttagggcatagtgatg | 48486 | GUCCUUUAGGGCAUAGUGAUG | 817372 | UCACUAUGCCCUAAAGGACAC | 817373 |
| CALM3 | NM_005184.1 | 0.16 | 538 | caggtcaattatgaagagtttgt | 129585 | GGUCAAUUAUGAAGAGUUUGU | 817374 | AAACUCUUCAUAAUUGACCUG | 817375 |
| DIAPH1 | NM_005219.2 | 0.2 | 885 | cagccgctgctggatggattaaa | 116504 | GCCGCUGCUGGAUGGAUUAAA | 817376 | UAAUCCAUCCAGCAGCGGCUG | 817377 |
| NCL | NM_005381.2 | 0.036 | 1276 | gagcgagatgcgagaacactttt | 33529 | GCGAGAUGCGAGAACACUUUU | 817378 | AAGUGUUCUCGCAUCUCGCUC | 817379 |
| TOB1 | NM_005749.2 | 0.097 | 588 | accaagttcggctctaccaaaat | 136820 | CAAGUUCGGCUCUACCAAAAU | 817380 | UUUGGUAGAGCCGAACUUGGU | 817381 |
| MADH2 | NM_005901.2 | 0.088 | 1456 | aagccgtctatcagctaactaga | 133708 | GCCGUCUAUCAGCUAACUAGA | 817382 | UAGUUAGCUGAUAGACGGCUU | 817383 |
| GNB2L1 | NM_006098.3 | 0.16 | 380 | caccaccacgaggcgatttgtgg | 125242 | CCACCACGAGGCGAUUUGUGG | 817384 | ACAAAUCGCCUCGUGGUGGUG | 817385 |
| PPP2R5A | NM_006243.2 | 0.18 | 890 | gagtatgtttcaactaatcgtgg | 298747 | GUAUGUUUCAACUAAUCGUGG | 817386 | ACGAUUAGUUGAAACAUACUC | 817387 |
| HYOU1 | NM_006389.2 | 0.1 | 427 | tccaaaggctacgctacgttact | 85421 | CAAAGGCUACGCUACGUUACU | 817388 | UAACGUAGCGUAGCCUUUGGA | 817389 |
| KHDRBS1 | NM_006559.1 | 0.15 | 1248 | aaggctacgaaggctattacagc | 19623 | GGCUACGAAGGCUAUUACAGC | 817390 | UGUAAUAGCCUUCGUAGCCUU | 817391 |
| METAP2 | NM_006838.2 | 0.084 | 738 | atgccggtgacacaacagtatta | 186558 | GCCGGUGACACAACAGUAUUA | 817392 | AUACUGUUGUGUCACCGGCAU | 817393 |
| CALM1 | NM_006888.2 | 0.4 | 519 | tacgtcacgtcatgacaaactta | 129581 | CGUCACGUCAUGACAAACUUA | 817394 | AGUUUGUCAUGACGUGACGUA | 817395 |
| TOPBP1 | NM_007027.2 | 0.3 | 1047 | atgcaagttgcgtaagtgaatca | 126366 | GCAAGUUGCGUAAGUGAAUCA | 817396 | AUUCACUUACGCAACUUGCAU | 817397 |
| PIAS1 | NM_016166.1 | 0.37 | 1704 | gccttacgacttacaaggattag | 35123 | CUUACGACUUACAAGGAUUAG | 817398 | AAUCCUUGUAAGUCGUAAGGC | 817399 |
| NMT1 | NM_021079.3 | 0.25 | 1025 | ctgggctgcgaccaatggaaaca | 215454 | GGGCUGCGACCAAUGGAAACA | 817400 | UUUCCAUUGGUCGCAGCCCAG | 817401 |
| PPP2R4 | NM_021131.3 | 0.28 | 332 | cgctgactacatcggattcatcc | 298444 | CUGACUACAUCGGAUUCAUCC | 817402 | AUGAAUCCGAUGUAGUCAGCG | 817403 |
| MSH6 | NM_000179.1 | 0.17 | 3185 | tgcggcgactgttctataacttt | 16779 | CGGCGACUGUUCUAUAACUUU | 817404 | AGUUAUAGAACAGUCGCCGCA | 817405 |
| EIF4A1 | NM_001416.1 | 0.16 | 493 | tggccgtgtgtttgatatgctta | 25763 | GCCGUGUGUUUGAUAUGCUUA | 817406 | AGCAUAUCAAACACACGGCCA | 817407 |
| ATP2A2 | NM_001681.2 | 0.2 | 2836 | atccccatacccgatgacaatgg | 72769 | CCCCAUACCCGAUGACAAUGG | 817408 | AUUGUCAUCGGGUAUGGGGAU | 817409 |
| HNRPK | NM_002140.2 | 0.24 | 458 | ccccgagcgcatattgagtatca | 28586 | CCGAGCGCAUAUUGAGUAUCA | 817410 | AUACUCAAUAUGCGCUCGGGG | 817411 |
| MSN | NM_002444.2 | 0.058 | 1806 | agcgcattgacgaatttgagtct | 287504 | CGCAUUGACGAAUUUGAGUCU | 817412 | ACUCAAAUUCGUCAAUGCGCU | 817413 |
| MAPK6 | NM_002748.2 | 0.16 | 1854 | ttggcctgtacataacaactttg | 89971 | GGCCUGUACAUAACAACUUUG | 817414 | AAGUUGUUAUGUACAGGCCAA | 817415 |
| MAP2K3 | NM_002756.2 | 0.26 | 626 | ttctacggggcactattcagaga | 88970 | CUACGGGGCACUAUUCAGAGA | 817416 | UCUGAAUAGUGCCCCGUAGAA | 817417 |
| RDX | NM_002906.2 | 0.049 | 43 | atcaacgtaagagtaactacaat | 118854 | CAACGUAAGAGUAACUACAAU | 817418 | UGUAGUUACUCUUACGUUGAU | 817419 |
| BHLHB2 | NM_003670.1 | 0.31 | 1011 | gagaaaggatcggcgcaattaag | 1511 | GAAAGGAUCGGCGCAAUUAAG | 817420 | UAAUUGCGCCGAUCCUUUCUC | 817421 |
| RIPK2 | NM_003821.4 | 0.38 | 1028 | acctcaccgagcacgtatgatct | 99178 | CUCACCGAGCACGUAUGAUCU | 817422 | AUCAUACGUGCUCGGUGAGGU | 817423 |
| HSF1 | NM_005526.1 | 0.1 | 1137 | ccccgaccgccctcattgactcc | 5332 | CCGACCGCCCUCAUUGACUCC | 817424 | AGUCAAUGAGGGCGGUCGGGG | 817425 |
| POP4 | NM_006627.1 | 0.099 | 639 | gtgaacggtctgcgaagaagttc | 53540 | GAACGGUCUGCGAAGAAGUUC | 817426 | ACUUCUUCGCAGACCGUUCAC | 817427 |
| DDX18 | NM_006773.3 | 0.28 | 196 | ctgaccctatcggaaactcaaaa | 50595 | GACCCUAUCGGAAACUCAAAA | 817428 | UUGAGUUUCCGAUAGGGUCAG | 817429 |
| DDX24 | NM_020414.3 | 0.24 | 2275 | aaggagcgaatccgtttagctcg | 50750 | GGAGCGAAUCCGUUUAGCUCG | 817430 | AGCUAAACGGAUUCGCUCCUU | 817431 |
| IFNGR1 | NM_000416.1 | 0.18 | 220 | taccgtagaggtaaagaactatg | 124614 | CCGUAGAGGUAAAGAACUAUG | 817432 | UAGUUCUUUACCUCUACGGUA | 817433 |
| AK1 | NM_000476.1 | 0.25 | 517 | agcggctggagacctattacaag | 71895 | CGGCUGGAGACCUAUUACAAG | 817434 | UGUAAUAGGUCUCCAGCCGCU | 817435 |
| SERPINE1 | NM_000602.1 | 0.4 | 786 | cacgcccgatggccattactacg | 183595 | CGCCCGAUGGCCAUUACUACG | 817436 | UAGUAAUGGCCAUCGGGCGUG | 817437 |
| IGF2R | NM_000876.1 | 0.14 | 6206 | acggagtctcgtactatataaat | 206285 | GGAGUCUCGUACUAUAUAAAU | 817438 | UUAUAUAGUACGAGACUCCGU | 817439 |
| RRM1 | NM_001033.2 | 0.25 | 1880 | cagggcccatacgaaacctatga | 226213 | GGGCCCAUACGAAACCUAUGA | 817440 | AUAGGUUUCGUAUGGGCCCUG | 817441 |
| CSNK1G2 | NM_001319.5 | 0.34 | 228 | gagctccgcctaggaaagaatct | 77703 | GCUCCGCCUAGGAAAGAAUCU | 817442 | AUUCUUUCCUAGGCGGAGCUC | 817443 |
| CDC42 | NM_001791.2 | 0.15 | 160 | tcctgatatcctacacaacaaac | 108667 | CUGAUAUCCUACACAACAAAC | 817444 | UUGUUGUGUAGGAUAUCAGGA | 817445 |
| CDH2 | NM_001792.2 | 0.23 | 1737 | atgccggtaccatgttgacaaca | 139854 | GCCGGUACCAUGUUGACAACA | 817446 | UUGUCAACAUGGUACCGGCAU | 817447 |
| CLU | NM_001831.1 | 0.14 | 161 | aagtaagtacgtcaataaggaaa | 303887 | GUAAGUACGUCAAUAAGGAAA | 817448 | UCCUUAUUGACGUACUUACUU | 817449 |
| CSNK1A1 | NM_001892.3 | 0.16 | 796 | agggctaaaggctgcaacaaaga | 77640 | GGCUAAAGGCUGCAACAAAGA | 817450 | UUUGUUGCAGCCUUUAGCCCU | 817451 |
| CSNK1D | NM_001893.3 | 0.29 | 657 | gtcgcatcgaatacattcattca | 77650 | CGCAUCGAAUACAUUCAUUCA | 817452 | AAUGAAUGUAUUCGAUGCGAC | 817453 |
| CTNNA1 | NM_001903.2 | 0.22 | 653 | aacgttccgatcctctatactgc | 290175 | CGUUCCGAUCCUCUAUACUGC | 817454 | AGUAUAGAGGAUCGGAACGUU | 817455 |
| DDR1 | NM_001954.3 | 0.32 | 1176 | ggctatgcaggtccactgtaaca | 78045 | CUAUGCAGGUCCACUGUAACA | 817456 | UUACAGUGGACCUGCAUAGCC | 817457 |
| PLAGL1 | NM_002656.2 | 0.31 | 2951 | ctcctgctacccaaaataccttt | 35406 | CCUGCUACCCAAAAUACCUUU | 817458 | AGGUAUUUUGGGUAGCAGGAG | 817459 |
| PPM1B | NM_002706.3 | 0.18 | 1479 | ttgctggcaagcgtaatgttatt | 162107 | GCUGGCAAGCGUAAUGUUAUU | 817460 | UAACAUUACGCUUGCCAGCAA | 817461 |
| PTPRF | NM_002840.2 | 0.14 | 3438 | aggttcccgactcctataagtca | 37154 | GUUCCCGACUCCUAUAAGUCA | 817462 | ACUUAUAGGAGUCGGGAACCU | 817463 |
| RPA1 | NM_002945.2 | 0.12 | 1784 | tacaacgacgagtctcgaattaa | 19815 | CAACGACGAGUCUCGAAUUAA | 817464 | AAUUCGAGACUCGUCGUUGUA | 817465 |
| SMARCA4 | NM_003072.2 | 0.18 | 3624 | tgcgtatcgcggctttaaatacc | 11695 | CGUAUCGCGGCUUUAAAUACC | 817466 | UAUUUAAAGCCGCGAUACGCA | 817467 |
| YY1 | NM_003403.3 | 0.15 | 904 | gacgacgactacattgaacaaac | 13964 | CGACGACUACAUUGAACAAAC | 817468 | UUGUUCAAUGUAGUCGUCGUC | 817469 |
| USP7 | NM_003470.1 | 0.25 | 1096 | ttgtcgagtgttgctcgataatg | 190309 | GUCGAGUGUUGCUCGAUAAUG | 817470 | UUAUCGAGCAACACUCGACAA | 817471 |
| IKBKG | NM_003639.2 | 0.44 | 1164 | aggcccaggcggatatctacaag | 254494 | GCCCAGGCGGAUAUCUACAAG | 817472 | UGUAGAUAUCCGCCUGGGCCU | 817473 |
| IQGAP1 | NM_003870.3 | 0.12 | 2335 | tcgctgccgtggatacttagttc | 121324 | GCUGCCGUGGAUACUUAGUUC | 817474 | ACUAAGUAUCCACGGCAGCGA | 817475 |
| CREBBP | NM_004380.1 | 0.19 | 2205 | gaggtcgcgtttacataaacaag | 2467 | GGUCGCGUUUACAUAAACAAG | 817476 | UGUUUAUGUAAACGCGACCUC | 817477 |
| CSNK1G3 | NM_004384.1 | 0.31 | 1252 | cccaccgcaggacgttcaaatgc | 77737 | CACCGCAGGACGUUCAAAUGC | 817478 | AUUUGAACGUCCUGCGGUGGG | 817479 |
| PPARBP | NM_004774.2 | 0.17 | 489 | atgttacatcacgtcagatatgt | 36486 | GUUACAUCACGUCAGAUAUGU | 817480 | AUAUCUGACGUGAUGUAACAU | 817481 |
| SFPQ | NM_005066.1 | 0.15 | 1533 | atggcacgtttgagtacgaatat | 39201 | GGCACGUUUGAGUACGAAUAU | 817482 | AUUCGUACUCAAACGUGCCAU | 817483 |
| ROCK1 | NM_005406.1 | 0.14 | 1201 | agcaatcgtagatacttatcttc | 99228 | CAAUCGUAGAUACUUAUCUUC | 817484 | AGAUAAGUAUCUACGAUUGCU | 817485 |
| TP53BP1 | NM_005657.1 | 0.28 | 396 | gacggtaatagtgggttcaatga | 20875 | CGGUAAUAGUGGGUUCAAUGA | 817486 | AUUGAACCCACUAUUACCGUC | 817487 |
| NCOR1 | NM_006311.2 | 0.36 | 6785 | cccgctcaccagggagtataagc | 33678 | CGCUCACCAGGGAGUAUAAGC | 817488 | UUAUACUCCCUGGUGAGCGGG | 817489 |
| TADA3L | NM_006354.2 | 0.1 | 1233 | ctgaccgaactggacactaaaga | 12451 | GACCGAACUGGACACUAAAGA | 817490 | UUUAGUGUCCAGUUCGGUCAG | 817491 |
| CTCF | NM_006565.1 | 0.25 | 1786 | cagtgtgattacgcttgtagaca | 2613 | GUGUGAUUACGCUUGUAGACA | 817492 | UCUACAAGCGUAAUCACACUG | 817493 |
| RUVBL2 | NM_006666.1 | 0.086 | 218 | gccggtcgggcagtccttattgc | 100117 | CGGUCGGGCAGUCCUUAUUGC | 817494 | AAUAAGGACUGCCCGACCGGC | 817495 |
| PRKDC | NM_006904.6 | 0.18 | 11629 | atgtataagggcgctaatcgtac | 205894 | GUAUAAGGGCGCUAAUCGUAC | 817496 | ACGAUUAGCGCCCUUAUACAU | 817497 |
| CNOT7 | NM_013354.4 | 0.12 | 844 | ttgagatccttcgattgtttttt | 2347 | GAGAUCCUUCGAUUGUUUUUU | 817498 | AAAACAAUCGAAGGAUCUCAA | 817499 |
| GSK3A | NM_019884.2 | 0.14 | 1477 | accccgtcctcacaagctttaac | 84369 | CCCGUCCUCACAAGCUUUAAC | 817500 | UAAAGCUUGUGAGGACGGGGU | 817501 |
| XRCC5 | NM_021141.2 | 0.83 | 1202 | tggccatagttcgatatgcttat | 20327 | GCCAUAGUUCGAUAUGCUUAU | 817502 | AAGCAUAUCGAACUAUGGCCA | 817503 |
| APP | NM_000484.1 | 0.14 | 1604 | ggcctcgtcacgtgttcaatatg | 128991 | CCUCGUCACGUGUUCAAUAUG | 817504 | UAUUGAACACGUGACGAGGCC | 817505 |
| ABCC5 | NM_005688.1 | 0.38 | 4297 | ttctaggctccgataggattatg | 70574 | CUAGGCUCCGAUAGGAUUAUG | 817506 | UAAUCCUAUCGGAGCCUAGAA | 817507 |
| NR2F2 | NM_021005.2 | 0.17 | 1106 | ctcgtacctgtccggatatattt | 8466 | CGUACCUGUCCGGAUAUAUUU | 817508 | AUAUAUCCGGACAGGUACGAG | 817509 |
| CDK4 | NM_000075.2 | 0.32 | 388 | ttcgtgaggtggctttactgagg | 76146 | CGUGAGGUGGCUUUACUGAGG | 817510 | UCAGUAAAGCCACCUCACGAA | 817511 |
| CLN2 | NM_000391.2 | 0.14 | 643 | tccgtaagcgatacaacttgacc | 183713 | CGUAAGCGAUACAACUUGACC | 817512 | UCAAGUUGUAUCGCUUACGGA | 817513 |
| AAMP | NM_001087.2 | 0.27 | 300 | tagcgaggtcacctttgcattgc | 177411 | GCGAGGUCACCUUUGCAUUGC | 817514 | AAUGCAAAGGUGACCUCGCUA | 817515 |
| ACLY | NM_001096.2 | 0.19 | 1229 | ggcatcgtgagagcaattcgaga | 71580 | CAUCGUGAGAGCAAUUCGAGA | 817516 | UCGAAUUGCUCUCACGAUGCC | 817517 |
| ADD1 | NM_001119.3 | 0.14 | 255 | aggtacttcgaccgagtagatga | 115154 | GUACUUCGACCGAGUAGAUGA | 817518 | AUCUACUCGGUCGAAGUACCU | 817519 |
| FLNA | NM_001456.1 | 0.13 | 4967 | aaccatgacggcacgtatacagt | 114101 | CCAUGACGGCACGUAUACAGU | 817520 | UGUAUACGUGCCGUCAUGGUU | 817521 |
| IL13RA1 | NM_001560.2 | 0.077 | 497 | accagtcccgacactaactatac | 244096 | CAGUCCCGACACUAACUAUAC | 817522 | AUAGUUAGUGUCGGGACUGGU | 817523 |
| IL18 | NM_001562.2 | 0.19 | 679 | ttcatcatacgaaggatactttc | 167828 | CAUCAUACGAAGGAUACUUUC | 817524 | AAGUAUCCUUCGUAUGAUGAA | 817525 |
| ARF6 | NM_001663.2 | 0.32 | 730 | gccgctctggcggcattactaca | 108231 | CGCUCUGGCGGCAUUACUACA | 817526 | UAGUAAUGCCGCCAGAGCGGC | 817527 |
| ITGB4BP | NM_002212.2 | 0.13 | 82 | gagcttcgttcgagaacaactgt | 56942 | GCUUCGUUCGAGAACAACUGU | 817528 | AGUUGUUCUCGAACGAAGCUC | 817529 |
| MYBL2 | NM_002466.2 | 0.093 | 975 | caggagcccatcggtacagatct | 7221 | GGAGCCCAUCGGUACAGAUCU | 817530 | AUCUGUACCGAUGGGCUCCUG | 817531 |
| NME2 | NM_002512.1 | 0.045 | 485 | aactggttgactacaagtcttgt | 8178 | CUGGUUGACUACAAGUCUUGU | 817532 | AAGACUUGUAGUCAACCAGUU | 817533 |
| RAP1A | NM_002884.1 | 0.072 | 529 | aagaacggccaaggttttgcact | 110570 | GAACGGCCAAGGUUUUGCACU | 817534 | UGCAAAACCUUGGCCGUUCUU | 817535 |
| RPA2 | NM_002946.3 | 0.15 | 289 | aacagtggattcgaaagctatgg | 19817 | CAGUGGAUUCGAAAGCUAUGG | 817536 | AUAGCUUUCGAAUCCACUGUU | 817537 |
| SDC2 | NM_002998.3 | 0.27 | 1182 | aggcacctactaaggagttttat | 121066 | GCACCUACUAAGGAGUUUUAU | 817538 | AAAACUCCUUAGUAGGUGCCU | 817539 |
| SDC4 | NM_002999.2 | 0.14 | 338 | cccaccgaacccaagaaactaga | 121072 | CACCGAACCCAAGAAACUAGA | 817540 | UAGUUUCUUGGGUUCGGUGGG | 817541 |
| TCF12 | NM_003205.2 | 0.17 | 1972 | cgcttacgcgtgcgggatattaa | 41688 | CUUACGCGUGCGGGAUAUUAA | 817542 | AAUAUCCCGCACGCGUAAGCG | 817543 |
| TIMP1 | NM_003254.1 | 0.12 | 364 | accgcagcgaggagtttctcatt | 186907 | CGCAGCGAGGAGUUUCUCAUU | 817544 | UGAGAAACUCCUCGCUGCGGU | 817545 |
| TRA1 | NM_003299.1 | 0.22 | 827 | taggacggggaacgacaattacc | 102862 | GGACGGGGAACGACAAUUACC | 817546 | UAAUUGUCGUUCCCCGUCCUA | 817547 |
| VCL | NM_003373.2 | 0.09 | 2111 | gtctcggctgctcgtatcttact | 120054 | CUCGGCUGCUCGUAUCUUACU | 817548 | UAAGAUACGAGCAGCCGAGAC | 817549 |
| CXCR4 | NM_003467.1 | 0.37 | 90 | tggaggggatcagtatatacact | 124591 | GAGGGGAUCAGUAUAUACACU | 817550 | UGUAUAUACUGAUCCCCUCCA | 817551 |
| SUCLG1 | NM_003849.1 | 0.5 | 127 | ttctcggcaacatctctatgttg | 110935 | CUCGGCAACAUCUCUAUGUUG | 817552 | ACAUAGAGAUGUUGCCGAGAA | 817553 |
| MBD2 | NM_003927.3 | 0.1 | 902 | ctgcgaaacgatcctctcaatca | 20354 | GCGAAACGAUCCUCUCAAUCA | 817554 | AUUGAGAGGAUCGUUUCGCAG | 817555 |
| USP13 | NM_003940 | 0.19 | 2338 | ggcactacgagcaacgaataata | 154317 | CACUACGAGCAACGAAUAAUA | 817556 | UUAUUCGUUGCUCGUAGUGCC | 817557 |
| OSMR | NM_003999.1 | 0.31 | 3240 | ctcccccgaccgagaatagcagc | 34501 | CCCCCGACCGAGAAUAGCAGC | 817558 | UGCUAUUCUCGGUCGGGGGAG | 817559 |
| GMFB | NM_004124.2 | 0.12 | 290 | gacaacctcgcttcattgtgtat | 117415 | CAACCUCGCUUCAUUGUGUAU | 817560 | ACACAAUGAAGCGAGGUUGUC | 817561 |
| EPHA2 | NM_004431.2 | 0.29 | 1535 | gtggaagtacgaggtcacttacc | 81348 | GGAAGUACGAGGUCACUUACC | 817562 | UAAGUGACCUCGUACUUCCAC | 817563 |
| USP11 | NM_004651.2 | 0.089 | 643 | ttcagccataccgattctattgg | 188785 | CAGCCAUACCGAUUCUAUUGG | 817564 | AAUAGAAUCGGUAUGGCUGAA | 817565 |
| USP9X | NM_004652.2 | 0.057 | 3790 | gtgggtcgttacagctagtattt | 190527 | GGGUCGUUACAGCUAGUAUUU | 817566 | AUACUAGCUGUAACGACCCAC | 817567 |
| USP14 | NM_005151.2 | 0.1 | 583 | tggcttcagcgcagtatattact | 188886 | GCUUCAGCGCAGUAUAUUACU | 817568 | UAAUAUACUGCGCUGAAGCCA | 817569 |
| USP10 | NM_005153.1 | 0.22 | 1353 | ccccgtgggctgatcaataaagg | 188770 | CCGUGGGCUGAUCAAUAAAGG | 817570 | UUUAUUGAUCAGCCCACGGGG | 817571 |
| USP8 | NM_005154.2 | 0.22 | 3283 | gagctcgacgggattctctaaaa | 190459 | GCUCGACGGGAUUCUCUAAAA | 817572 | UUAGAGAAUCCCGUCGAGCUC | 811573 |
| SDCBP | NM_005625.3 | 0.13 | 259 | tcctatccctcacgatggaaatc | 114865 | CUAUCCCUCACGAUGGAAAUC | 817574 | UUUCCAUCGUGAGGGAUAGGA | 817575 |
| CAPZA1 | NM_006135.1 | 0.16 | 101 | atgacgttcggctactacttaat | 113977 | GACGUUCGGCUACUACUUAAU | 817576 | UAAGUAGUAGCCGAACGUCAU | 817577 |
| CAPZA2 | NM_006136.2 | 0.15 | 762 | gacaatgtcggacactactttca | 114011 | CAAUGUCGGACACUACUUUCA | 817578 | AAAGUAGUGUCCGACAUUGUC | 817579 |
| NFE2L2 | NM_006164.2 | 0.21 | 324 | ttcgctcagttacaactagatga | 7735 | CGCUCAGUUACAACUAGAUGA | 817580 | AUCUAGUUGUAACUGAGCGAA | 817581 |
| USP16 | NM_006447.1 | 0.19 | 1205 | tggtggtgaactaactagtatga | 189070 | GUGGUGAACUAACUAGUAUGA | 817582 | AUACUAGUUAGUUCACCACCA | 817583 |
| LIM | NM_006457.1 | 0.19 | 1333 | ctccgatgtgcgcccattgtaac | 125275 | CCGAUGUGCGCCCAUUGUAAC | 817584 | UACAAUGGGCGCACAUCGGAG | 817585 |
| ADD3 | NM_016824.1 | 0.14 | 1732 | gacaatcgaacgtaaacaacaag | 121236 | CAAUCGAACGUAAACAACAAG | 817586 | UGUUGUUUACGUUCGAUUGUC | 817587 |
| MAP2K2 | NM_030662.2 | 0.16 | 670 | gtgacggggagatcagcatttgc | 88959 | GACGGGGAGAUCAGCAUUUGC | 817588 | AAAUGCUGAUCUCCCCGUCAC | 817589 |
| ADH5 | NM_000671.2 | 0.11 | 685 | ggcatttcaaccggttatggtgc | 155944 | CAUUUCAACCGGUUAUGGUGC | 817590 | ACCAUAACCGGUUGAAAUGCC | 817591 |
| ANXA1 | NM_000700.1 | 0.11 | 946 | ctcgccataaggcattgatcagg | 137873 | CGCCAUAAGGCAUUGAUCAGG | 817592 | UGAUCAAUGCCUUAUGGCGAG | 817593 |
| FOLR1 | NM_000802.2 | 0.32 | 259 | ttcctacctatatagattcaact | 179653 | CCUACCUAUAUAGAUUCAACU | 817594 | UUGAAUCUAUAUAGGUAGGAA | 817595 |
| POLR2B | NM_000938.1 | 0.16 | 2960 | ccctctcgtatgactattggtca | 36387 | CUCUCGUAUGACUAUUGGUCA | 817596 | ACCAAUAGUCAUACGAGAGGG | 817597 |
| CRIP2 | NM_001312.2 | 0.25 | 90 | gtgcgacaagaccgtgtacttcg | 156364 | GCGACAAGACCGUGUACUUCG | 817598 | AAGUACACGGUCUUGUCGCAC | 817599 |
| POLR2C | NM_002694.2 | 0.11 | 153 | ttcgattcggagggtcttcatcg | 36408 | CGAUUCGGAGGGUCUUCAUCG | 817600 | AUGAAGACCCUCCGAAUCGAA | 817601 |
| POLR2E | NM_002695.2 | 0.098 | 40 | gacgtaccggctctggaaaatcc | 36425 | CGUACCGGCUCUGGAAAAUCC | 817602 | AUUUUCCAGAGCCGGUACGUC | 817603 |
| RFC3 | NM_002915.2 | 0.38 | 120 | tgggacggctggactatcacaag | 38420 | GGACGGCUGGACUAUCACAAG | 817604 | UGUGAUAGUCCAGCCGUCCCA | 817605 |
| RFC4 | NM_002916.3 | 0.19 | 957 | ttcaaagcgctactcgattaaca | 38466 | CAAAGCGCUACUCGAUUAACA | 817606 | UUAAUCGAGUAGCGCUUUGAA | 817607 |
| SSB | NM_003142.2 | 0.1 | 276 | aggttgaaccgtctaacaacaga | 48020 | GUUGAACCGUCUAACAACAGA | 817608 | UGUUGUUAGACGGUUCAACCU | 817609 |
| HSPA9B | NM_004134.4 | 0.19 | 948 | ggccttgctacggcacattgtga | 85288 | CCUUGCUACGGCACAUUGUGA | 817610 | ACAAUGUGCCGUAGCAAGGCC | 817611 |
| FANCG | NM_004629.1 | 0.28 | 1405 | ctgctagttgaggccttgaatgt | 16276 | GCUAGUUGAGGCCUUGAAUGU | 817612 | AUUCAAGGCCUCAACUAGCAG | 817613 |
| POLR2K | NM_005034.2 | 0.17 | 182 | gtggatacagaataatgtacaag | 36435 | GGAUACAGAAUAAUGUACAAG | 817614 | UGUACAUUAUUCUGUAUCCAC | 817615 |
| PRCP | NM_005040.2 | 0.043 | 970 | tggcaatggtggactatccttat | 187207 | GCAAUGGUGGACUAUCCUUAU | 817616 | AAGGAUAGUCCACCAUUGCCA | 817617 |
| HSPA5 | NM_005347.2 | 0.21 | 1292 | gtggctcgactcgaattccaaag | 85234 | GGCUCGACUCGAAUUCCAAAG | 817618 | UUGGAAUUCGAGUCGAGCCAC | 817619 |
| POLR2H | NM_006232.2 | 0.099 | 262 | gtcatagctagtaccttgtatga | 159134 | CAUAGCUAGUACCUUGUAUGA | 817620 | AUACAAGGUACUAGCUAUGAC | 817621 |
| POLR2I | NM_006233.4 | 0.11 | 145 | acgcgtgccggaactgtgattac | 9602 | GCGUGCCGGAACUGUGAUUAC | 817622 | AAUCACAGUUCCGGCACGCGU | 817623 |
| POLR2J | NM_006234.3 | 0.15 | 359 | gcgctttcgggtggccataaaag | 36430 | GCUUUCGGGUGGCCAUAAAAG | 817624 | UUUAUGGCCACCCGAAAGCGC | 817625 |
| RFC5 | NM_007370.3 | 0.23 | 941 | aggggttggcactgcatgatatc | 38487 | GGGUUGGCACUGCAUGAUAUC | 817626 | UAUCAUGCAGUGCCAACCCCU | 817627 |
| TGFB1I1 | NM_015927.3 | 0.16 | 532 | gacttccgcgttcaaaaccatct | 112567 | CUUCCGCGUUCAAAACCAUCU | 817628 | AUGGUUUUGAACGCGGAAGUC | 817629 |
| PRKWNK1 | NM_018979.1 | 0.1 | 631 | gccgtgggaatgtctaacgatgg | 97669 | CGUGGGAAUGUCUAACGAUGG | 817630 | AUCGUUAGACAUUCCCACGGC | 817631 |
| POLR2F | NM_021974.2 | 0.052 | 105 | tggcgacgactttgatgatgtgg | 36427 | GCGACGACUUUGAUGAUGUGG | 817632 | ACAUCAUCAAAGUCGUCGCCA | 817633 |
| NME1 | NM_000269.2 | 0.045 | 185 | agcgttttgagcagaaaggattc | 94844 | CGUUUUGAGCAGAAAGGAUUC | 817634 | AUCCUUUCUGCUCAAAACGCU | 817635 |
| PEA15 | NM_003768.2 | 0.15 | 406 | ccgtcctgacctactcactatgg | 134551 | GUCCUGACCUACUCACUAUGG | 817636 | AUAGUGAGUAGGUCAGGACGG | 817637 |
| ARHGDIA | NM_004309.3 | 0.17 | 438 | ttccgggttaaccgagagatagt | 129087 | CCGGGUUAACCGAGAGAUAGU | 817638 | UAUCUCUCGGUUAACCCGGAA | 817639 |
| ESRRA | NM_004451.3 | 0.3 | 1029 | ggccttcgctgaggacttagtcc | 3459 | CCUUCGCUGAGGACUUAGUCC | 817640 | ACUAAGUCCUCAGCGAAGGCC | 817641 |
| CAV1 | NM_001753.3 | 0.13 | 702 | cagtgcatcagccgtgtctattc | 289819 | GUGCAUCAGCCGUGUCUAUUC | 817642 | AUAGACACGGCUGAUGCACUG | 817643 |
| MKI67 | NM_002417.2 | 0.17 | 558 | cacgtcgtgtctcaagatctagc | 91497 | CGUCGUGUCUCAAGAUCUAGC | 817644 | UAGAUCUUGAGACACGACGUG | 817645 |
| CDKN1B | NM_004064.2 | 0.41 | 929 | ctgcaaccgacgattcttctact | 219267 | GCAACCGACGAUUCUUCUACU | 817646 | UAGAAGAAUCGUCGGUUGCAG | 817647 |
| ERBB2 | NM_004448.1 | 0.17 | 3386 | aaggggctggctccgatgtattt | 81940 | GGGGCUGGCUCCGAUGUAUUU | 817648 | AUACAUCGGAGCCAGCCCCUU | 817649 |
| MXI1 | NM_005962.2 | 0.14 | 920 | cacagcagcctgccgagtattgg | 33013 | CAGCAGCCUGCCGAGUAUUGG | 817650 | AAUACUCGGCAGGCUGCUGUG | 817651 |

### INDUSTRIAL APPLICABILITY

In view of the foregoing, the polynucleotide of the present invention not only has a high RNA interference effect on its target gene, but also has a very small risk of causing RNA interference against a gene unrelated to the target gene, so that the polynucleotide of the present invention can cause RNA interference specifically only to the target gene whose expression is to be inhibited. Thus, the present invention is preferred for use in, e.g., tests and therapies using RNA interference, and is particularly effective in performing RNA interference in higher animals such as mammals, especially humans.

Incidentally, the sequence listing of the present application contains information on 817651 sequences. Its electronic file is too large in size (near 200 MB), making it difficult or impossible to handle the file depending on the computer environment used. Thus, the electric file was divided into two parts so that it became easier to handle. YCT1039 sequence listing (1) contains bibliographic data and information on SEQ ID NOs: 1 to 70000, while YCT1039 sequence listing (2) contains information on SEQ ID NOs: 700001 to 817651.

1. A polynucleotide for causing RNA interference against a target gene selected from the genes of a target organism, which has at least a double-stranded region, wherein one strand in the double-stranded region consists of a base sequence homologous to a prescribed sequence which is contained in the base sequences of the target gene and which conforms to the following rules (a) to (d):
(a) The 3′ end base is adenine, thymine or uracil;
(b) The 5′ end base is guanine or cytosine;
(c) A 7-base sequence from the 3′ end is rich in one or more types of bases selected from the group consisting of adenine, thymine and uracil; and
(d) The number of bases is within a range that allows RNA interference to occur without causing cytotoxicity, and
wherein the other strand in the double-stranded region consists of a base sequence having a sequence complementary to the base sequence homologous to the prescribed sequence.

2. The polynucleotide according to claim 1, wherein at least 80% of bases in the base sequence homologous to the prescribed sequence corresponds to the base sequence of the prescribed sequence.

3. The polynucleotide according to claim 1, wherein, in the rule (c), at least three bases among the seven bases are one or more types of bases selected from the group consisting of adenine, thymine and uracil.

4. The polynucleotide according to claim 1, wherein, in the rule (d), the number of bases is 13 to 28.

5. The polynucleotide according to claim 1, wherein the prescribed sequence further conforms to the following rule (e):
(e) A sequence in which 10 or more bases of guanine or cytosine are continuously present is not contained.

6. The polynucleotide according to claim 5, wherein the prescribed sequence further conforms to the following rule (f):
(f) A sequence sharing at least 90% homology with the prescribed sequence is not contained in the base sequences of genes other than the target gene among all gene sequences of the target organism.

7. The polynucleotide according to claim 6, wherein the prescribed sequence consists of the base sequence shown in any of SEQ ID NOs: 47 to 817081.

8. The polynucleotide according to claim 6, wherein the prescribed sequence is any of the sequences listed in the column “Target Sequence” of FIG. 46.

9. The polynucleotide according to claim 6, which has any of the base sequences shown in SEQ ID NOs: 817102 to 817651.

10. The polynucleotide according to claim 1, which is a double-stranded polynucleotide.

11. The polynucleotide according to claim 10, wherein
one strand of the double-stranded polynucleotide consists of a base sequence having an overhanging portion at the 3′ end of the base sequence homologous to the prescribed sequence, and the other strand of the double-stranded polynucleotide consists of a base sequence having an overhanging portion at the 3′ end of the sequence complementary to the base sequence homologous to the prescribed sequence.

12. The polynucleotide according to claim 1, which is a single-stranded polynucleotide having a hairpin structure, wherein the single-stranded polynucleotide has a loop segment linking the 3′ end of one strand in the double-stranded region and the 5′ end of the other strand in the double-stranded region.

13. A method for selecting a polynucleotide to be introduced into an expression system for a target gene whose expression is to be inhibited, wherein the polynucleotide has at least a double-stranded region, wherein one strand in the double-stranded region consists of a base sequence homologous to a prescribed sequence which is contained in the base sequences of the target gene and which conforms to the following rules (a) to (f):
(a) The 3′ end base is adenine, thymine or uracil;
(b) The 5′ end base is guanine or cytosine;
(c) A 7-base sequence from the 3′ end is rich in one or more types of bases selected from the group consisting of adenine, thymine and uracil;
(d) The number of bases is within a range that allows RNA interference to occur without causing cytotoxicity;
(e) A sequence in which 10 or more bases of guanine or cytosine are continuously present is not contained; and
(f) A sequence sharing at least 90% homology with the prescribed sequence is not contained in the base sequences of genes other than the target gene among all gene sequences of the target organism, and
wherein the other strand in the double-stranded region consists of a base sequence having a sequence complementary to the base sequence homologous to the prescribed sequence.

14. A method for selecting a polynucleotide according to claim 13, wherein a polynucleotide having a sequence, wherein the base sequence homologous to the prescribed sequence of the target gene contains mismatches of at least 3 bases against the base sequences of genes other than the target gene, and for which there is only a minimum number of other genes having a base sequence containing the mismatches of at least 3 bases, is further selected from the selected polynucleotides.

15. A method for inhibiting gene expression, which comprises introducing the polynucleotide according to claim 1 into an expression system for a target gene whose expression is to be inhibited, thereby inhibiting the expression of the target gene.

16. A method for inhibiting gene expression, which comprises introducing a polynucleotide selected by the method according to claim 13 into an expression system for a target gene whose expression is to be inhibited, thereby inhibiting the expression of the target gene.

17. The method for inhibiting gene expression according to claim 15, wherein the expression is inhibited to 50% or below.

18. A pharmaceutical composition which comprises a pharmaceutically effective amount of the polynucleotide according to claim 1.

19. The pharmaceutical composition according to claim 18, which is for use in treating or preventing the diseases listed in the column “Related Disease” of FIG. 46.

20. The pharmaceutical composition according to claim 18, which is for use in treating or preventing diseases related to the genes listed in the column “Gene Name” of FIG. 46.

21. The pharmaceutical composition according to claim 18, which is for use in treating or preventing a disease in which a gene belonging to any of the following 1) to 9) is involved:
1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or
9) G protein-coupled receptor or a G protein-coupled receptor-related gene.

22. The pharmaceutical composition according to claim 18, which comprises a polynucleotide targeting the base sequence shown in any of SEQ ID NOs listed in the column “SEQ ID NO (human)” or “SEQ ID NO (mouse)” of FIG. 46.

23. The pharmaceutical composition according to claim 18, which is for use in treating or preventing diseases related to the genes listed in the column “Gene Name” of Table 1.

24. The pharmaceutical composition according to claim 18, which is for use in treating or preventing any cancer selected from bladder cancer, breast cancer, colorectal cancer, gastric cancer, hepatoma, lung cancer, melanoma, ovarian cancer, pancreas cancer, prostate cancer, oral cancer, skin cancer, and thyroid gland cancer.

25. The pharmaceutical composition according to claim 18, which comprises a polynucleotide having any of the base sequences shown in SEQ ID NOs: 817102 to 817651.

26. A composition for inhibiting gene expression to inhibit the expression of a target gene, which comprises the polynucleotide according to claim 1.

27. The composition for inhibiting gene expression according to claim 26, wherein the target gene is related to any of the diseases listed in the column “Related Disease” of FIG. 46.

28. The composition for inhibiting gene expression according to claim 26, wherein the target gene is any of the genes listed in the column “Gene Name” of FIG. 46.

29. The composition for inhibiting gene expression according to claim 26, wherein the target gene is a gene belonging to any of the following 1) to 9):
1) an apoptosis-related gene;
2) phosphatase or a phosphatase activity-related gene;
3) a cell cycle-related gene;
4) a receptor-related gene;
5) an ion channel-related gene;
6) a signal transduction system-related gene;
7) kinase or a kinase activity-related gene;
8) a transcription regulation-related gene; or
9) G protein-coupled receptor or a G protein-coupled receptor-related gene.

30. The composition for inhibiting gene expression according to claim 26, wherein the target gene is any of the genes listed in the column “Gene Name” of Table 1.

31. The composition for inhibiting gene expression according to claim 26, wherein the target gene is related to any cancer selected from bladder cancer, breast cancer, colorectal cancer, gastric cancer, hepatoma, lung cancer, melanoma, ovarian cancer, pancreas cancer, prostate cancer, oral cancer, skin cancer, and thyroid gland cancer.

32. A method for treating or preventing the diseases listed in the column “Related Disease” of FIG. 46, which comprises administering a pharmaceutically effective amount of the polynucleotide according to claim 1.
